(19) United States
(12) Patent Application Publication
(10) Pub. No.: US 2025/0320214 A1
(43) Pub. Date: Oct. 16, 2025

(54) ORGANIC COMPOUNDS

(71) Applicant: INTRA-CELLULAR THERAPIES, INC., New York, NY (US)

(72) Inventors: Peng LI, New Milford, NJ (US); Robert E. DAVIS, San Diego, CA (US); Gretchen SNYDER, New York, NY (US); Allen A. FIENBERG, New York, NY (US)

(21) Appl. No.: 18/847,126

(22) PCT Filed: Mar. 14, 2023
(86) PCT No.: PCT/US2023/064349

§ 371 Date: Sep. 13, 2024

Related U.S. Application Data
(60) Provisional application No. 63/269,305, filed on Mar. 14, 2022.

(51) Int. Cl. C07D 471/16; A61K 31/437; A61K 31/4985; A61K 31/517; C07D 471/04

(52) U.S. Cl. CPC C07D 471/16; A61K 31/437; A61K 31/4985; A61K 31/517; C07D 471/04

(57) ABSTRACT

The invention relates to particular substituted heterocycle fused gamma-carbolines, in free, solid, pharmaceutically acceptable salt and/or substantially pure form as described herein, pharmaceutical compositions thereof, and methods of use in the treatment of diseases involving the 5-HT_{2A} receptor, the serotonin transporter (SERT), pathways involving the dopamine D₁ and D₂ receptor signaling system, and/or the μ-opioid receptor.

### CROSS-REFERENCE TO RELATED APPLICATIONS

This application is an international application which claims priority to, and the benefit of, U.S. Provisional Application Ser. No. 63/269,305, filed on Mar. 14, 2022, the contents of which are hereby incorporated by reference in its entirety.

### TECHNICAL FIELD

The invention relates to particular substituted heterocycle fused gamma-carbolines, in free, solid, pharmaceutically acceptable salt and/or substantially pure form as described herein, pharmaceutical compositions thereof, and methods of use in the treatment of diseases involving the 5-HT_{2A} receptor, the serotonin transporter (SERT), pathways involving dopamine D₁ and/or D₂ receptor signaling systems, and/or the μ-opioid receptor, e.g., diseases or disorders such as anxiety, psychosis, schizophrenia, sleep disorders, sexual disorders, migraine, conditions associated with pain (including cephalic pain, neuropathic pain, and as an acute analgesic), fibromyalgia, chronic fatigue, social phobias, gastrointestinal disorders such as dysfunction of the gastrointestinal tract motility and obesity; depression and mood disorders, such as those associated with psychosis or Parkinson's disease; psychosis such as schizophrenia associated with depression; bipolar disorder, drug dependencies, such as opioid dependency and alcohol dependency, drug withdrawal symptoms; obsessive-compulsive disorder (OCD), obsessive-compulsive personality disorder (OCPD), and related disorders; and other psychiatric and neurological conditions, as well as to combinations with other agents. In some embodiments, the disease or disorders may include treatment-resistant depression, cocaine dependency, and/or amphetamine dependency, opioid use disorder and the symptoms of opioid withdrawal.

### BACKGROUND

Substituted heterocycle fused gamma-carbolines are known to be agonists or antagonists of 5-HT₂ receptors, particularly 5-HT_{2A} receptors, in treating central nervous system disorders. These compounds have been disclosed in U.S. Pat. Nos. 6,548,493; 7,238,690; 6,552,017; 6,713,471; 7,183,282; U.S. RE39,680, and U.S. RE39,679, as novel compounds useful for the treatment of disorders associated with 5-HT_{2A} receptor modulation such as obesity, anxiety, depression, psychosis, schizophrenia, sleep disorders, sexual disorders migraine, conditions associated with cephalic pain, social phobias, gastrointestinal disorders such as dysfunction of the gastrointestinal tract motility, and obesity. U.S. Pat. Nos. 8,309,722, and 7,081,455, also disclose methods of making substituted heterocycle fused gamma-carbolines and uses of these gamma-carbolines as serotonin agonists and antagonists useful for the control and prevention of central nervous system disorders such as addictive behavior and sleep disorders.

In addition. U.S. Pat. No. 8,598,119 discloses use of particular substituted heterocycle fused gamma-carbolines for the treatment of a combination of psychosis and depressive disorders as well as sleep, depressive and/or mood disorders in patients with psychosis or Parkinson's disease. In addition to disorders associated with psychosis and/or depression, this patent discloses or claims use of these compounds at a low dose to selectively antagonize 5-HT_{2A} receptors without affecting or minimally affecting dopamine D₂ receptors, thereby making the compounds useful for the treatment of sleep disorders without the side effects associated with high occupancy of the dopamine D₂ pathways or side effects of other pathways (e.g., GABA_{A} receptors) associated with conventional sedative-hypnotic agents (e.g., benzodiazepines), including but not limited to the development of drug dependency, muscle hypotonia, weakness, headache, blurred vision, vertigo, nausea, vomiting, epigastric distress, diarrhea, joint pains, and chest pains. U.S. Pat. No. 8,648,077 also discloses methods of preparing toluenesulfonic acid addition salt crystals of these substituted heterocycle fused gamma-carbolines.

In addition, recent evidence shows that the aforementioned substituted fused heterocycle gamma carbolines may operate, in part, through NMDA receptor antagonism via mTOR1 signaling, in a manner similar to that of ketamine. Ketamine is a selective NMDA receptor antagonist. Ketamine acts through a system that is unrelated to the common psychogenic monoamines (serotonin, norepinephrine and dopamine), and this is a major reason for its much more rapid effects in treating depression or anxiety. Ketamine directly antagonizes extrasynaptic glutamatergic NMDA receptors, which also indirectly results in activation of AMPA-type glutamate receptors. The downstream effects involve the brain-derived neurotrophic factor (BDNF) and mTORC1 kinase pathways. Similar to ketamine, recent evidence suggests that compounds related to those of the present disclosure enhance both NMDA and AMPA-induced currents in rat medial prefrontal cortex pyramidal neurons via activation of D1 receptors, and that this is associated with increased mTORC1 signaling. WO 2019/178484, and US 2021/0060009, each of which is hereby incorporated by reference in their entireties, disclose such effects for certain substituted fused heterocycle gamma-carbolines, and useful therapeutic indications related thereto.

The publication US 2017/319580, and U.S. Pat. Nos. 10,245,260 and 10,799,500, the contents of each of which are hereby incorporated by reference in their entireties, disclose novel oxo-metabolites of the compounds disclosed in the above-mentioned publications. These new oxo-metabolites retain much of the unique pharmacologic activity of the parent compounds, including serotonin receptor inhibition, SERT inhibition, and dopamine receptor modulation. However, these oxo-metabolites were found to unexpectedly also show significant activity at mu-opioid receptors. Further indications for use, and methods of treatment, for such compounds have also been disclosed in, e.g., U.S. Pat. No. 11,376,249, US 2021/0093634, US 2022/0088014, US 2022/0184072, and PCT/US2022/078177, the contents of each of which are hereby incorporated by reference in their entireties.

Analogs of these novel compounds have also been disclosed, for example, in U.S. Pat. Nos. 10,961,245, 10,906,906, 11,427,587, US 2021/0009592, and US 2022/0048910, the contents of each of which are hereby incorporated by reference in their entireties.

The Compound of Formula A, shown below, for example, is a potent serotonin 5-HT_{2A} receptor antagonist and mu-opioid receptor partial agonist or biased agonist. This compound also interacts with dopamine receptors, particular the dopamine D1 receptors.

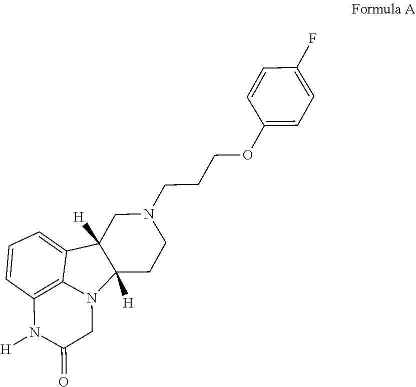 [SMILES: O=C1CN2c3c(cccc3[C@@H]3CN(CCCOc4ccc(F)cc4)CC[C@@H]32)N1]

It is also believed that the Compound of Formula A, via its D1 receptor activity, may also enhance NMDA and AMPA mediated signaling through the mTOR pathway. The Compound of Formula A is thus useful for the treatment or prophylaxis of central nervous system disorders, but there is a need in the art additional compounds having this unique biochemical and pharmacological profile, especially those which may have subtly altered pharmacologic or pharmacokinetic profiles compared to the Compound of Formula A.

Obsessive-compulsive disorder (OCD) and related disorders, have become highly prevalent and are difficult to treat. OCD is estimated to affect about 2.3% of people at some point in their lives, and during a given year, it is estimated than 1.2% of people worldwide suffer from the disorder. Half of people who suffer from OCD begin to show symptoms before the age of 20, which can seriously affect their ability to obtain an adequate and effective education. Without effective treatment, however, the disease can last for decades. The mainstay of pharmacologic OCD treatment is with selective serotonin reuptake inhibitors (SSRIs). A second line of therapy is with antipsychotic agents, such as clomipramine, risperidone, quetiapine and olanzapine. A significant number of patients either do not respond to these agents, or cannot handle the side effects caused by these agents. More recently, it has been reported that the opioid analgesic tramadol may be effective in treating OCD. Opioids operate by an entirely different pathway from traditional OCD treatment agents, so they offer the possibility of treatment for people who cannot take the traditional serotonergic agents or for whom these agents are ineffective. However, strong opioid agents can be addictive, and their use may be contraindicated in some patients. There thus remains an urgent need for new treatments for pain, OCD and other disorders.

Drug dependency disorders, such as opioid use disorder (OUD), are another group of disorders which are difficult to successfully treat. Opioid overdoses claim approximately 100 lives in the United States every day, and the opioid epidemic continues to grow in the United States. Methadone, buprenorphine, and naltrexone are the most frequently used treatments for OUD. Methadone is a mu-opioid receptor (MOP) agonist, buprenorphine is an MOP partial agonist, and naltrexone is an MOP antagonist. Each of these agents has had limited success, and long-term adherence to prescribed therapies for OUD remains low. In addition, these treatments often exacerbate common co-morbidities associated with OUD, such as mood and anxiety disorders, which further increases the risk of remission. Abrupt opioid abuse withdrawal (i.e., going “cold turkey”) is also associated with severe side effects, including dysphoria, depression and anxiety, and the common treatment agents do not address these problems, and may make them worse.

There is thus an urgent need for new compounds having a combination of pharmacological activity at a combination of one or more of the serotonin 5-HT2A, dopamine D1, dopamine D2, and mu-opioid receptors and/or the serotonin transporter.

### BRIEF SUMMARY

In a first aspect, the present disclosure relates to a compound (Compound I) of Formula I:

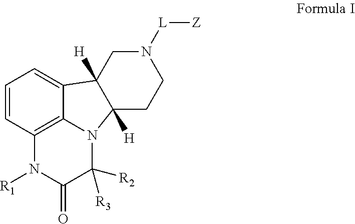

wherein:
R¹ is H, C₁₋₆alkyl, —C(O)—O—C(R^{a})(R^{b})(R^{c}), —C(O)—O—CH₂—O—C(R^{a})(R^{b})(R^{c}) or —C(R⁶)(R⁷)—O—C(O)—R⁸;
R² and R³ are independently selected from H, D, C₁₋₆alkyl (e.g., methyl), C₁₋₆alkoxy (e.g., methoxy), halo (e.g., F), cyano, or hydroxy;
   or wherein R² and R³ and the carbon to which they are attached collectively form a group —CH₂CH₂—, or
   wherein R² and R³ and the carbon to which they are attached are absent;
L is C₁₋₆alkylene (e.g., ethylene, propylene, or butylene), C₁₋₆alkoxy (e.g., propoxy or butoxy), C₂₋₃alkoxyC₁₋₃alkylene (e.g., CH₂CH₂OCH₂), C₁₋₄alkylamino or N—(C₁₋₆alkyl)-C₁₋₆alkylamino (e.g., propylamino or N-methylpropylamino), C₁₋₆alkylthio (e.g., —CH₂CH₂CH₂S—), C₁₋₆alkylsulfonyl (e.g., —CH₂CH₂CH₂S(O)₂—), or —C₁₋₆alkyl-C(O)— (e.g., 4-butanoyl), each of which is optionally substituted with one or more R⁴ moieties;
each R⁴ is independently selected from C₁₋₆alkyl (e.g., methyl), C₁₋₆alkoxy (e.g., methoxy), halo (e.g., F), cyano, hydroxy, amino (—NH₂), C₁₋₆alkylaryl (e.g., benzyl), C₁₋₆alkoxyaryl (e.g., benzyloxy), aryloxy (e.g., phenoxy), —C(O)-aryl, —C(O)—C₁₋₆alkyl, —C(O)OH, —C(O)NH₂, —C(O)NH(C₁₋₆alkyl), and C(O)N(C₁₋₆alkyl)(C₁₋₆alkyl);
Z is selected from aryl (e.g., phenyl) and heteroaryl (e.g., thiophenyl, furanyl, pyridyl, indazolyl, benzimidazolyl, benzisoxazolyl), methyl or OH, each optionally substituted with one or more R⁴ moieties, optionally wherein Z is unsubstituted;
R⁸ is —C(R^{a})(R^{b})(R^{c}), —O—C(R^{a})(R^{b})(R^{c}), or —N(R^{d})(R^{e});
R^{a}, R^{b} and R^{c} are each independently selected from H and C₁₋₂₄alkyl;
R^{d} and R^{e} are each independently selected from H and C₁₋₂₄alkyl;
R⁶ and R⁷ are each independently selected from H, C₁₋₆alkyl, carboxy and C₁₋₆alkoxycarbonyl;
provided that if Z is aryl or heteroaryl at least one group R⁴ on substituent Z is selected from amino (—NH₂), C₁₋₆alkylaryl (e.g., benzyl), C₁₋₆alkoxyaryl (e.g., benzyloxy), aryloxy (e.g., phenoxy), —C(O)-aryl, —C(O)—C₁₋₆alkyl, —C(O)OH, —C(O)NH₂, —C(O)NH(C₁₋₆alkyl), and C(O)N(C₁₋₆alkyl)(C₁₋₆alkyl);
in free or salt form (e.g., pharmaceutically acceptable salt form), for example in an isolated or purified free or salt form (e.g., pharmaceutically acceptable salt form).

The present disclosure provides additional exemplary embodiments of the Compound of Formula I, in free or salt form (e.g., pharmaceutically acceptable salt form), for example in an isolated or purified free or salt form (e.g., pharmaceutically acceptable salt form), including:
1.1 Compound I, wherein R¹ is H;
1.2 Compound I, wherein R¹ is C₁₋₆alkyl, e.g., methyl;
1.3 Compound I, wherein R¹ is —C(O)—O—C(R^{a})(R^{b})(R^{c});
1.4 Compound 1.3, wherein R^{a} is H and R^{b} and R^{c} are each independently selected from C₁₋₂₄alkyl, e.g., C₁₋₂₀alkyl, C₅₋₂₀alkyl, C₉₋₁₈alkyl, C₁₀₋₁₆alkyl, or C₁₁alkyl, C₁₂alkyl, C₁₃alkyl, C₁₄alkyl, C₁₅alkyl or C₁₆alkyl;
1.5 Compound 1.3, wherein R^{a} and R^{b} are H and R^{c} is C₁₋₂₄alkyl, e.g., C₁₋₂₀alkyl, C₅₋₂₀alkyl, C₉₋₁₈alkyl, C₁₀₋₁₆alkyl, or C₁₁alkyl, C₁₂alkyl, C₁₃alkyl, C₁₄alkyl, C₁₅alkyl or C₁₆alkyl;
1.6 Compound 1.3, wherein R^{a}, R^{b} and R^{c} are each independently selected from C₁₋₂₄alkyl, e.g., C₁₋₂₀alkyl, C₅₋₂₀alkyl, C₉₋₁₈alkyl, C₁₀₋₁₆alkyl, or C₁₁alkyl, C₁₂alkyl, C₁₃alkyl, C₁₄alkyl, C₁₅alkyl or C₁₆alkyl;
1.7 Compound 1.3, wherein R^{a}, R^{b} and R^{c} are each H;
1.8 Compound 1.3, wherein R^{a} and R^{b} are H and R^{c} is C₁₀₋₁₄alkyl (e.g., R^{c} is CH₃(CH₂)₁₀ or CH₃(CH₂)₁₄);
1.9 Compound I, wherein R¹ is —C(O)—O—CH₂—O—C(R^{a})(R^{b})(R^{c}).
1.10 Compound 1.9, wherein R^{a} is H and R^{b} and R^{c} are each independently selected from C₁₋₂₄alkyl, e.g., C₁₋₂₀alkyl, C₅₋₂₀alkyl, C₉₋₁₈alkyl, C₁₀₋₁₆alkyl, or C₁₁alkyl, C₁₂alkyl, C₁₃alkyl, C₁₄alkyl, C₁₅alkyl or C₁₆alkyl;
1.11 Compound 1.9, wherein R^{a} and R^{b} are H and R^{c} is C₁₋₂₄alkyl, e.g., C₁₋₂₀alkyl, C₅₋₂₀alkyl, C₉₋₁₈alkyl, C₁₀₋₁₆alkyl, or C₁₁alkyl, C₁₂alkyl, C₁₃alkyl, C₁₄alkyl, C₁₅alkyl or C₁₆alkyl;
1.12 Compound 1.9, wherein R^{a}, R^{b} and R^{c} are each independently selected from C₁₋₂₄alkyl, e.g., C₁₋₂₀alkyl, C₅₋₂₀alkyl, C₉₋₁₈alkyl, C₁₀₋₁₆alkyl, or C₁₁alkyl, C₁₂alkyl, C₁₃alkyl, C₁₄alkyl, C₁₅alkyl or C₁₆alkyl;
1.13 Compound 1.9, wherein R^{a}, R^{b} and R^{c} are each H;
1.14 Compound I, wherein R¹ is —C(R⁶)(R⁷)—O—C(O)—R⁸, and R⁸ is —C(R^{a})(R^{b})(R^{c});
1.15 Compound I, wherein R¹ is —C(R⁶)(R⁷)—O—C(O)—R⁸, and R⁸ is —O—C(R^{a})(R^{b})(R^{c});
1.16 Compound 1.14 or 1.15, wherein R^{a} is H and R^{b} and R^{c} are each independently selected from C₁₋₂₄alkyl, e.g., C₁₋₂₀alkyl, C₅₋₂₀alkyl, C₉₋₁₈alkyl, C₁₀₋₁₆alkyl, or C₁₁alkyl, C₁₂alkyl, C₁₃alkyl, C₁₄alkyl, C₁₅alkyl or C₁₆alkyl;
1.17 Compound 1.14 or 1.15, wherein R^{a} and R^{b} are H and R^{c} is C₁₋₂₄alkyl, e.g., C₁₋₂₀alkyl, C₅₋₂₀alkyl, C₉₋₁₈alkyl, C₁₀₋₁₆alkyl, or C₁₁alkyl, C₁₂alkyl, C₁₃alkyl, C₁₄alkyl, C₁₅alkyl or C₁₆alkyl;
1.18 Compound 1.14 or 1.15, wherein R^{a}, R^{b} and R^{c} are each independently selected from C₁₋₂₄alkyl, e.g., C₁₋₂₀alkyl, C₅₋₂₀alkyl, C₉₋₁₈alkyl, C₁₀₋₁₆alkyl, or C₁₁alkyl, C₁₂alkyl, C₁₃alkyl, C₁₄alkyl, C₁₅alkyl or C₁₆alkyl;
1.19 Compound 1.14 or 1.15, wherein R^{a}, R^{b} and R^{c} are each H;
1.20 Any of compounds 1.14-1.19, wherein R⁶ is H, and R⁷ is C₁₋₃alkyl (e.g., R⁷ is methyl or isopropyl), and R⁸ is C₁₀₋₁₄alkyl (e.g., R⁸ is CH₃(CH₂)₁₀ or CH₃(CH₂)₁₄);
1.21 Compound 1, wherein R¹ is —C(R⁶)(R⁷)—O—C(O)—R⁸, and R⁸ is —N(R^{d})(R^{e});
1.22 Compound 1.21, wherein R^{d} is H and R^{e} is independently selected from C₁₋₂₄alkyl, e.g., C₁₋₂₀alkyl, C₅₋₂₀alkyl, C₉₋₁₈alkyl, C₁₀₋₁₆alkyl, or C₁₁alkyl, C₁₂alkyl, C₁₃alkyl, C₁₄alkyl, C₁₅alkyl or C₁₆alkyl;
1.23 Compound 1.21, wherein R^{d} and R^{e} are each independently selected from C₁₋₂₄alkyl, e.g., C₁₋₂₀alkyl, C₅₋₂₀alkyl, C₉₋₁₈alkyl, C₁₀₋₁₆alkyl, or C₁₁alkyl, C₁₂alkyl, C₁₃alkyl, C₁₄alkyl, C₁₅alkyl or C₁₆alkyl;
1.24 Compound 1.21, wherein R^{d} and R^{e} are each H;
1.25 Any of Compounds 1.14-1.24, wherein R⁶ is H and R⁷ is H;
1.26 Any of Compounds 1.14-1.24, wherein R⁶ is C₁₋₆alkyl and R⁷ is C₁₋₆alkyl;
1.27 Any of Compounds 1.14-1.24, wherein R⁶ is H and R⁷ is C₁₋₆alkyl;
1.28 Any of Compounds 1.14-1.24, wherein R⁶ is H and R⁷ is carboxy;
1.29 Any of Compounds 1.14-1.24, wherein R⁶ is H and R⁷ is C₁₋₆alkoxycarbonyl, e.g., ethoxycarbonyl or methoxycarbonyl;
1.30 Compound I, or any of 1.1-1.29, wherein R² and R³ are H;
1.31 Compound I, or any of 1.1-1.29, wherein R² is H and R³ is D;
1.32 Compound I, or any of 1.1-1.29, wherein R² and R³ are D;
1.33 Compound I, or any of 1.1-1.32, wherein L is C₁₋₆alkylene (e.g., ethylene, propylene, or butylene), C₁₋₆alkoxy (e.g., propoxy), C₂₋₃alkoxyC₁₋₃alkylene (e.g., CH₂CH₂OCH₂) C₁₋₆alkylamino (e.g., propylamino or N-methylpropylamino), or C₁₋₆alkylthio (e.g., —CH₂CH₂CH₂S—), each of which is optionally substituted with one or more R⁴ moieties;
1.34 Compound 1.33, wherein L is unsubstituted C₁₋₆alkylene (e.g., ethylene, propylene, or butylene);
1.35 Compound 1.33, wherein L is C₁₋₆alkylene (e.g., ethylene, propylene, or butylene), substituted with one or more R⁴ moieties;
1.36 Compound 1.33, wherein L is unsubstituted C₁₋₆alkyoxy (e.g., propoxy or butoxy);
1.37 Compound 1.33, wherein L is C₁₋₆alkoxy (e.g., propoxy or butoxy), substituted with one or more R⁴ moieties;
1.38 Compound 1.33, wherein L is unsubstituted C₂₋₃alkoxyC₁₋₃alkylene (e.g., CH₂CH₂OCH₂);
1.39 Compound 1.33, wherein L is C₂₋₃alkoxyC₁₋₃alkylene (e.g., CH₂CH₂OCH₂), substituted with one or more R⁴ moieties;
1.40 Compound I, or any of 1.1-1.39, wherein R¹, R² and R³ are each H;
1.41 Compound I, or any of 1.1-1.40, wherein L is —(CH₂)ₙ—X—, and wherein n is an integer selected from 2, 3 and 4, and X is selected from —O—, —S—, —NH—, —N(C₁₋₆alkyl)-, —CH₂—, and —C(O)—;
1.42 Compound 1.41, wherein L is —(CH₂)ₙ—X—, and wherein n is an integer selected from 2, 3 and 4, and X is —O—;
1.43 Compound 1.41, wherein L is —(CH₂)ₙ—X—, and wherein n is 3, and X is selected from —O—, —S—, —NH—, —N(C₁₋₆alkyl)- (e.g., —N(CH₃)—), and —C(O)—;
1.44 Compound 1.41, wherein L is —(CH₂)ₙ—X—, and wherein n is 3, and X is O;
1.45 Compound 1.41, wherein L is —(CH₂)ₙ—X—, and wherein n is 3, and X is —C(O)—;
1.46 Compound I, or any of 1.1-1.45, wherein Z is aryl (e.g., phenyl), optionally substituted with one or more R⁴ moieties;
1.47 Compound 1.46, wherein Z is aryl (e.g., phenyl), substituted with one or more R⁴ moieties;
1.48 Compound 1.47, wherein Z is phenyl substituted with one, two, three, or four R⁴ moieties;
1.49 Compound 1.47 or 1.48, wherein the one, two, three, or four R⁴ moieties are independently selected from methyl, methoxy, hydroxy, amino (—NH₂), halo (e.g., fluoro, chloro, bromo or iodo), cyano, benzyl, benzyloxy, phenoxy, benzoyl, acetyl, pivaloyl, —C(O)OH, —C(O)NH₂, —C(O)NH(CH₃), and C(O)N(CH₃)(CH₃);
1.50 Compound 1.48 or 1.49, wherein Z is phenyl substituted with one R⁴ moiety selected from methyl, methoxy, hydroxy, amino (—NH₂), halo (e.g., fluoro, chloro, bromo or iodo), cyano, benzyl, benzyloxy, phenoxy, benzoyl, acetyl, pivaloyl, —C(O)OH, —C(O)NH₂, —C(O)NH(CH₃), and C(O)N(CH₃)(CH₃);
1.51 Compound 1.48 or 1.49, wherein said Z is phenyl substituted with two R⁴ moieties each independently selected from methyl, amino (—NH₂), halo (e.g., fluoro, chloro, bromo or iodo) and cyano;
1.52 Compound 1, or any of 1.1-1.45, wherein Z is heteroaryl (e.g., thiophenyl, furanyl, pyridyl, indazolyl, benzimidazolyl, benzisoxazolyl), optionally substituted with one or more R⁴ moieties;
1.53 Compound 1.52, wherein said heteroaryl is a monocyclic 5-membered or 6-membered heteroaryl (e.g., pyridyl, pyrimidyl, pyrazinyl, thiophenyl, pyrrolyl, thiophenyl, furanyl, imidazolyl, oxazolyl, isoxazolyl, thiazolyl);
1.54 Compound 1.53, wherein said heteroaryl is selected from pyridyl, pyrimidinyl and pyrazinyl;
1.55 Compound 1.53, wherein said heteroaryl is selected from thiophenyl, furanyl, pyrrolyl, imidazolyl, oxazolyl, isoxazolyl, thiazolyl, triazolyl, and tetrazolyl;
1.56 Compound 1.53, wherein said heteroaryl is selected from thiophenyl and furanyl;
1.57 Compound 1.53, wherein said heteroaryl is thiophenyl (e.g., 5-substituted-2-thiophenyl or 4-substituted-3-thiophenyl);
1.58 Compound 1.53, wherein said heteroaryl is a bicyclic 9-membered or 10-membered heteroaryl (e.g., indolyl, isoindolyl, benzofuranyl, benzothiophenyl, indazolyl, benzimidazolyl, benzoxazolyl, benzisoxazolyl, benzothiazolyl, quinolinyl, isoquinolinyl, quinoxalinyl, quinazolinyl, benzodioxolyl, or 2-oxo-tetrahydroquinolinyl);
1.59 Compound 1.58, wherein said heteroaryl is selected from indazolyl, benzisoxazolyl, quinolinyl, quinazolinyl, benzodioxolyl, and 2-oxo-tetrahydroquinolinyl;
1.60 Compound 1.58, wherein said heteroaryl is selected from indazolyl, benzisoxazolyl, quinolinyl, quinazolinyl;
1.61 Compound 1.60, wherein said heteroaryl is indazolyl (e.g., 5-substituted or 6-substituted-3-indazolyl, 1-substituted-3-indazolyl, or 1,5- or 1,6-disubstituted-3-indazolyl);
1.62 Compound 1.61, wherein said indazolyl is a 1-(C₁₋₃alkyl)-5-halo-3-indazolyl, or a 1-(C₁₋₃alkyl)-6-halo-3-indazolyl), optionally wherein said C₁₋₃alkyl is methyl and/or said halo is fluoro;
1.63 Any of Compounds 1.52-1.62, wherein said heteroaryl is substituted with one, two, three, or four R⁴ moieties;
1.64 Compound 1.63, wherein the one, two three or four R⁴ moieties are independently selected from methyl, methoxy, ethoxy, hydroxy, amino (—NH₂), halo (e.g., fluoro, chloro, bromo or iodo), cyano, benzyl, benzyloxy, phenoxy, benzoyl, acetyl, pivaloyl, —C(O)OH, —C(O)NH₂, —C(O)NH(CH₃), and C(O)N(CH₃)(CH₃);
1.65 Compound 1.63, wherein said heteroaryl is substituted with one R⁴ moiety selected from methyl, methoxy, hydroxy, amino (—NH₂), halo (e.g., fluoro, chloro, bromo or iodo), cyano, benzyl, benzyloxy), phenoxy, benzoyl, acetyl, pivaloyl, —C(O)OH, —C(O)NH₂, —C(O)NH(CH₃), and C(O)N(CH₃)(CH₃);
1.66 Compound I, or any of 1.1-1.65, wherein R² and R³ and the carbon to which they are attached collectively form a group —CH₂CH₂—, e.g., wherein the Compound I is a compound according to the formula:

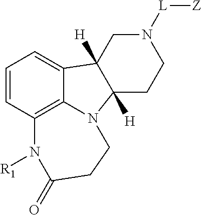

in free, or pharmaceutically acceptable salt form, wherein R¹, L and Z, are as defined in any preceding embodiment;
1.67 Compound I, or any of 1.1-1.65, wherein R² and R³ and the carbon to which they are attached are absent, e.g., wherein the Compound I is a compound according to the formula:

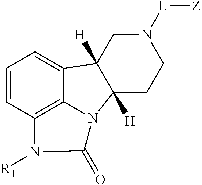

in free, or pharmaceutically acceptable salt form, wherein R¹, L and Z, are as defined in any preceding embodiment;
1.68 Compound I, or any of 1.1-1.67, wherein the compound is selected from the group consisting of:

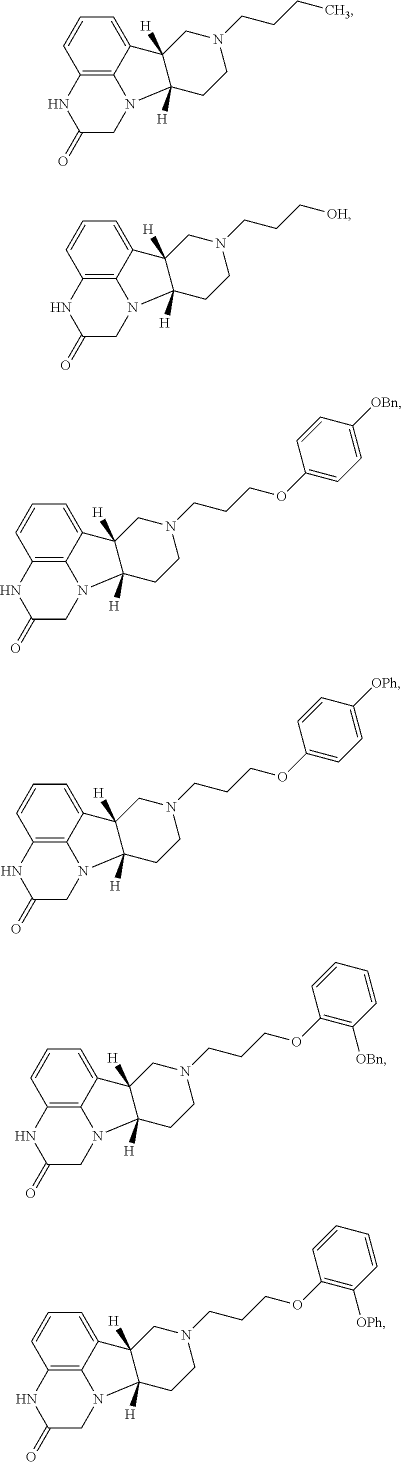 [SMILES: CCCCN1CC[C@H]2[C@@H](C1)c1cccc3c1N2CC(=O)N3 ; O=C1CN2c3c(cccc3[C@@H]3CN(CCCO)CC[C@@H]32)N1 ; O=C1CN2c3c(cccc3[C@@H]3CN(CCCOc4ccc(OCc5ccccc5)cc4)CC[C@@H]32)N1 ; O=C1CN2c3c(cccc3[C@@H]3CN(CCCOc4ccc(Oc5ccccc5)cc4)CC[C@@H]32)N1 ; O=C1CN2c3c(cccc3[C@@H]3CN(CCCOc4ccccc4OCc4ccccc4)CC[C@@H]32)N1 ; O=C1CN2c3c(cccc3[C@@H]3CN(CCCOc4ccccc4Oc4ccccc4)CC[C@@H]32)N1]

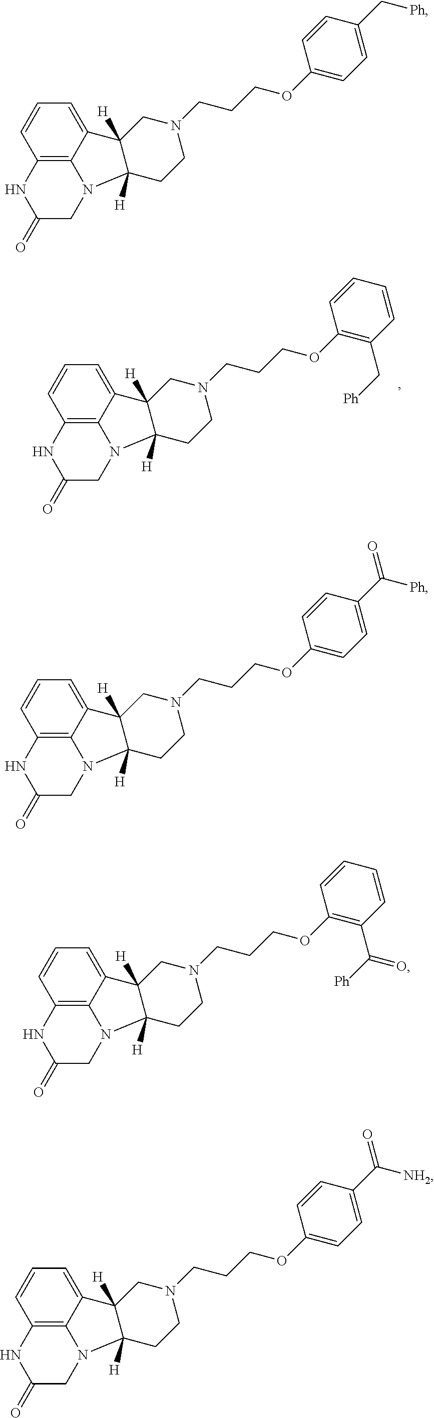 [SMILES: O=C1CN2c3c(cccc3[C@@H]3CN(CCCOc4ccc(Cc5ccccc5)cc4)CC[C@@H]32)N1 ; O=C1CN2c3c(cccc3[C@@H]3CN(CCCOc4ccccc4Cc4ccccc4)CC[C@@H]32)N1 ; O=C1CN2c3c(cccc3[C@@H]3CN(CCCOc4ccc(C(=O)c5ccccc5)cc4)CC[C@@H]32)N1 ; O=C1CN2c3c(cccc3[C@@H]3CN(CCCOc4ccccc4C(=O)c4ccccc4)CC[C@@H]32)N1 ; NC(=O)c1ccc(OCCCN2CC[C@H]3[C@@H](C2)c2cccc4c2N3CC(=O)N4)cc1]

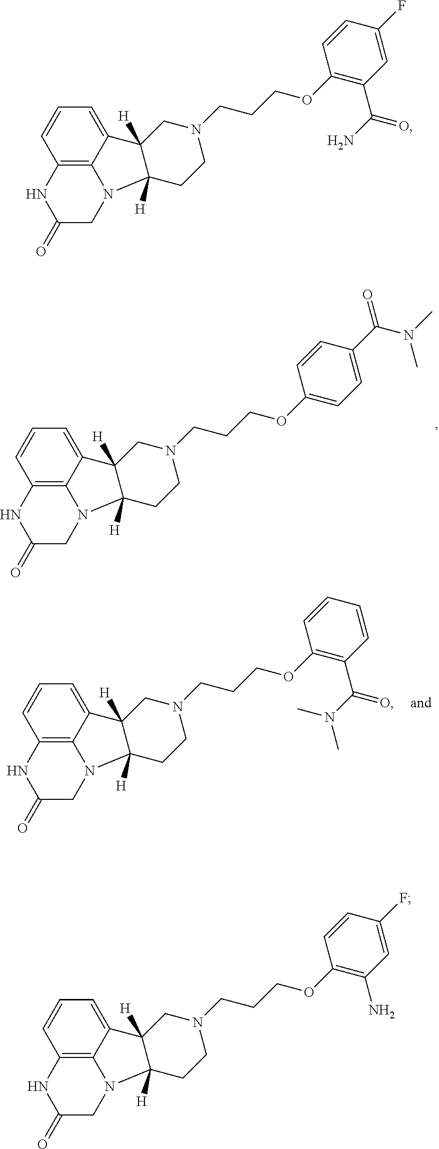 [SMILES: NC(=O)c1cc(F)ccc1OCCCN1CC[C@H]2[C@@H](C1)c1cccc3c1N2CC(=O)N3 ; CN(C)C(=O)c1ccc(OCCCN2CC[C@H]3[C@@H](C2)c2cccc4c2N3CC(=O)N4)cc1 ; CN(C)C(=O)c1ccccc1OCCCN1CC[C@H]2[C@@H](C1)c1cccc3c1N2CC(=O)N3 ; Nc1cc(F)ccc1OCCCN1CC[C@H]2[C@@H](C1)c1cccc3c1N2CC(=O)N3]

each independently in free or pharmaceutically acceptable salt or form;
1.69 Compound I, or any of 1.1-1.68, in free form;
1.70 Compound 1, or any of 1.1-1.68, in salt form, e.g., pharmaceutically acceptable salt form;
1.71 Compound I, or any of 1.1-1.68, wherein the compound is in acid addition salt form, for example, hydrochloric or toluenesulfonic acid addition salt form;
1.72 Compound I, or any of 1.1-1.71, in substantially pure diastereomeric form (i.e., substantially free from other diastereomers);
1.73 Compound I or any of 1.1-1.71, having a diastereomeric excess of greater than 70%, preferably greater than 80%, more preferably greater than 90% and most preferably greater than 95%;
1.74 Compound I or any of 1.1-1.73 in solid form, e.g., in crystal form;
1.75 Compound I or any of 1.1-1.74, in isolated or purified form (e.g., in at least 90% pure form, or at least 95% or at least 98% or at least 99%).

In another embodiment of the first aspect, the present disclosure provides a Compound of Formula II (Compound 2):

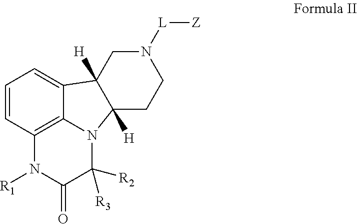

wherein:
R¹ is H, C₁₋₆alkyl, —C(O)—O—C(R^{a})(R^{b})(R^{c}), —C(O)—O—CH₂—O—C(R^{a})(R^{b})(R^{c}) or —C(R⁶)(R⁷)—O—C(O)—R⁸;
R² and R³ are independently selected from H, D, C₁₋₆alkyl (e.g., methyl), C₁₋₆alkoxy (e.g., methoxy), halo (e.g., F), cyano, or hydroxy;
   or wherein R² and R³ and the carbon to which they are attached collectively form a group —CH₂CH₂—, or
   wherein R² and R³ and the carbon to which they are attached are absent;
L is C₃ alkylene (e.g., propylene), C₃alkoxy (e.g., propoxy), C₃alkylsulfonyl (e.g., —CH₂CH₂CH₂S(O)₂—), or —C₂₋₄alkyl-C(O)— (e.g., 4-butanoyl); and
Z is selected from aryl (e.g., phenyl, naphthyl) and heteroaryl (e.g., bicyclic heteroaryl or monocyclic 5-membered heteroaryl), each of which is optionally substituted with one or more R⁴ moieties, optionally wherein Z is unsubstituted;
provided that L-Z is selected from:
   —CH₂CH₂CH₂O-Ph;
   —CH₂CH₂CH₂-(heteroaryl), wherein said heteroaryl is a bicyclic heteroaryl disubstituted with two R⁴ moieties;
   —CH₂CH₂CH₂-(heteroaryl), wherein said heteroaryl is a quinazolinyl substituted with one, two or three R⁴ moieties;
   —CH₂CH₂CH₂O-(aryl), wherein said aryl is phenyl or napthyl, and said aryl is monosubstituted with a hydroxy group;
   —CH₂CH₂CH₂O-(aryl), wherein said aryl is phenyl or napthyl, and said aryl is substituted with two or more R⁴ moieties;
   —(CH₂)n-C(O)-(heteroaryl), wherein n is an integer selected from 2, 3 and 4, and wherein said heteroaryl is a monocyclic 5-membered heteroaryl substituted with one to three R⁴ moieties;
   —CH₂CH₂CH₂S(O)₂-(aryl), and wherein said aryl is phenyl or napthyl, and said aryl is substituted by one or two R⁴ moieties; and
   —CH₂CH₂CH₂O-(aryl), wherein said aryl is phenyl or napthyl, and said aryl is substituted with one or more R⁴ moieties, provided that R² and R³ are F;
and wherein
each R⁴ is independently selected from C₁₋₆alkyl (e.g., methyl), C₁₋₆alkoxy (e.g., methoxy), halo (e.g., F), cyano, hydroxy, amino (—NH₂), C₁₋₆alkylaryl (e.g., benzyl), C₁₋₆alkoxyaryl (e.g., benzyloxy), aryloxy (e.g., phenoxy), —C(O)-aryl, —C(O)—C₁₋₆alkyl, —C(O)OH, —C(O)NH₂, —C(O)NH(C₁₋₆alkyl), and C(O)N(C₁₋₆alkyl)(C₁₋₆alkyl);
R⁸ is —C(R^{a})(R^{b})(R^{c}), —O—C(R^{a})(R^{b})(R^{c}), or —N(R^{d})(R^{e});
R^{a}, R^{b} and R^{c} are each independently selected from H and C₁₋₂₄alkyl;
R^{d} and R^{e} are each independently selected from H and C₁₋₂₄alkyl;
R⁶ and R⁷ are each independently selected from H, C₁₋₆alkyl, carboxy and C₁₋₆alkoxycarbonyl;
in free or salt form (e.g., pharmaceutically acceptable salt form), for example in an isolated or purified free or salt form (e.g., pharmaceutically acceptable salt form).

The present disclosure provides additional exemplary embodiments of the Compound of Formula II, in free or salt form (e.g., pharmaceutically acceptable salt form), for example in an isolated or purified free or salt form (e.g., pharmaceutically acceptable salt form), including:
2.1 Compound II, wherein R¹ is H;
2.2 Compound II, wherein R¹ is C₁₋₆alkyl, e.g., methyl;
2.3 Compound II, wherein R¹ is —C(O)—O—C(R^{a})(R^{b})(R^{c});
2.4 Compound 2.3, wherein R^{a} is H and R^{b} and R¹ are each independently selected from C₁₋₂₄alkyl, e.g., C₁₋₂₀alkyl, C₅₋₂₀alkyl, C₉₋₁₈alkyl, C₁₀₋₁₆alkyl, or C₁₁alkyl, C₁₂alkyl, C₁₃alkyl, C₁₄alkyl, C₁₅alkyl or C₁₆alkyl;
2.5 Compound 2.3, wherein R^{a} and R^{b} are H and R^{c} is C₁₋₂₄alkyl, e.g., C₁₋₂₀alkyl, C₅₋₂₀alkyl, C₉₋₁₈alkyl, C₁₀₋₁₆alkyl, or C₁₁alkyl, C₁₂alkyl, C₁₃alkyl, C₁₄alkyl, C₁₅alkyl or C₁₆alkyl;
2.6 Compound 2.3, wherein R^{a}, R^{b} and R^{c} are each independently selected from C₁₋₂₄alkyl, e.g., C₁₋₂₀alkyl, C₅₋₂₀alkyl, C₉₋₁₈alkyl, C₁₀₋₁₆alkyl, or C₁₁alkyl, C₁₂alkyl, C₁₃alkyl, C₁₄alkyl, C₁₅alkyl or C₁₆alkyl;
2.7 Compound 2.3, wherein R^{a}, R^{b} and R^{c} are each H;
2.8 Compound 2.3, wherein R^{a} and R^{b} are H and R^{c} is C₁₀₋₁₄alkyl (e.g., R^{c} is CH₃(CH₂)₁₀ or CH₃(CH₂)₁₄);
2.9 Compound II, wherein R¹ is —C(O)—O—CH₂—O—C(R^{a})(R^{b})(R^{c})
2.10 Compound 2.9, wherein R^{a} is H and R^{b} and R^{c} are each independently selected from C₁₋₂₄alkyl, e.g., C₁₋₂₀alkyl, C₅₋₂₀alkyl, C₉₋₁₈alkyl, C₁₀₋₁₆alkyl, or C₁₁alkyl, C₁₂alkyl, C₁₃alkyl, C₁₄alkyl, C₁₅alkyl or C₁₆alkyl;
2.11 Compound 2.9, wherein R^{a} and R^{b} are H and R^{c} is C₁₋₂₄alkyl, e.g., C₁₋₂₀alkyl, C₅₋₂₀alkyl, C₉₋₁₈alkyl, C₁₀₋₁₆alkyl, or C₁₁alkyl, C₁₂alkyl, C₁₃alkyl, C₁₄alkyl, C₁₅alkyl or C₁₆alkyl;
2.12 Compound 2.9, wherein R^{a}, R^{b} and R^{c} are each independently selected from C₁₋₂₄alkyl, e.g., C₁₋₂₀alkyl, C₅₋₂₀alkyl, C₉₋₁₈alkyl, C₁₀₋₁₆alkyl, or C₁₁alkyl, C₁₂alkyl, C₁₃alkyl, C₁₄alkyl, C₁₅alkyl or C₁₆alkyl;
2.13 Compound 2.9, wherein R^{a}, R^{b} and R^{c} are each H;
2.14 Compound II, wherein R¹ is —C(R⁶)(R⁷)—O—C(O)—R⁸, and R⁸ is —C(R^{a})(R^{b})(R^{c});
2.15 Compound II, wherein R¹ is —C(R⁶)(R⁷)—O—C(O)—R⁸, and R⁸ is —O—C(R^{a})(R^{b})(R^{c});
2.16 Compound 2.14 or 2.15, wherein R^{a} is H and R^{b} and R^{c} are each independently selected from C₁₋₂₄alkyl, e.g., C₁₋₂₀alkyl, C₅₋₂₀alkyl, C₉₋₁₈alkyl, C₁₀₋₁₆alkyl, or C₁₁alkyl, C₁₂alkyl, C₁₃alkyl, C₁₄alkyl, C₁₅alkyl or C₁₆alkyl;
2.17 Compound 2.14 or 2.15, wherein R^{a} and R^{b} are H and R^{c} is C₁₋₂₄alkyl, e.g., C₁₋₂₀alkyl, C₅₋₂₀alkyl, C₉₋₁₈alkyl, C₁₀₋₁₆alkyl, or C₁₁alkyl, C₁₂alkyl, C₁₃alkyl, C₁₄alkyl, C₁₅alkyl or C₁₆alkyl;
2.18 Compound 2.14 or 2.15, wherein R^{a}, R^{b} and R^{c} are each independently selected from C₁₋₂₄alkyl, e.g., C₁₋₂₀alkyl, C₅₋₂₀alkyl, C₉₋₁₈alkyl, C₁₀₋₁₆alkyl, or C₁₁alkyl, C₁₂alkyl, C₁₃alkyl, C₁₄alkyl, C₁₅alkyl or C₁₆alkyl;
2.19 Compound 2.14 or 2.15, wherein R^{a}, R^{b} and R^{c} are each H;
2.20 Any of compounds 2.14-2.19, wherein R⁶ is H, and R⁷ is C₁₋₃alkyl (e.g., R⁷ is methyl or isopropyl), and R⁸ is C₁₀₋₁₄alkyl (e.g., R⁸ is CH₃(CH₂)₁₀ or CH₃(CH₂)₁₄);
2.21 Compound II, wherein R¹ is —C(R⁶)(R⁷)—O—C(O)—R⁸, and R⁸ is —N(R^{d})(R^{e});
2.22 Compound 2.21, wherein R^{d} is H and R^{e} is independently selected from C₁₋₂₄alkyl, e.g., C₁₋₂₀alkyl, C₅₋₂₀alkyl, C₉₋₁₈alkyl, C₁₀₋₁₆alkyl, or C₁₁alkyl, C₁₂alkyl, C₁₃alkyl, C₁₄alkyl, C₁₅alkyl or C₁₆alkyl;
2.23 Compound 2.21, wherein R^{d} and R^{e} are each independently selected from C₁₋₂₄alkyl, e.g., C₁₋₂₀alkyl, C₅₋₂₀alkyl, C₉₋₁₈alkyl, C₁₀₋₁₆alkyl, or C₁₁alkyl, C₁₂alkyl, C₁₃alkyl, C₁₄alkyl, C₁₅alkyl or C₁₆alkyl;
2.24 Compound 2.21, wherein R^{d} and R^{e} are each H;
2.25 Any of Compounds 2.14-2.24, wherein R⁶ is H and R⁷ is H;
2.26 Any of Compounds 2.14-2.24, wherein R⁶ is C₁₋₆alkyl and R⁷ is C₁₋₆alkyl;
2.27 Any of Compounds 2.14-2.24, wherein R⁶ is H and R⁷ is C₁₋₆alkyl;
2.28 Any of Compounds 2.14-2.24, wherein R⁶ is H and R⁷ is carboxy;
2.29 Any of Compounds 2.14-2.24, wherein R⁶ is H and R⁷ is C₁₋₆alkoxycarbonyl, e.g., ethoxycarbonyl or methoxycarbonyl;
2.30 Compound II, or any of 2.1-2.29, wherein R² and R³ are H;
2.31 Compound II, or any of 2.1-2.29, wherein R² is H and R³ is D;
2.32 Compound 11, or any of 2.1-2.29, wherein R² and R³ are D;
2.33 Compound II, or any of 2.1-2.32, wherein L is —CH₂CH₂CH₂O-Ph;
2.34 Compound II, or any of 2.1-2.32, wherein L is —CH₂CH₂CH₂-(heteroaryl), wherein said heteroaryl is a bicyclic heteroaryl disubstituted with two R⁴ moieties, or wherein said heteroaryl is a quinazolinyl substituted with one, two or three R⁴ moieties;
2.35 Compound 2.34, wherein said heteroaryl is a bicyclic 9-membered or 10-membered heteroaryl (e.g., indolyl, isoindolyl, benzofuranyl, benzothiophenyl, indazolyl, benzimidazolyl, benzoxazolyl, benzisoxazolyl, benzothiazolyl, quinolinyl, isoquinolinyl, quinoxalinyl, quinazolinyl, benzodioxolyl, 2-oxo-tetrahydroquinolinyl);
2.36 Compound 2.34, wherein said heteroaryl is selected from indazolyl, benzisoxazolyl, quinolinyl, benzodioxolyl, and 2-oxo-tetrahydroquinolinyl;
2.37 Compound 2.34, wherein said heteroaryl is selected from indazolyl, benzisoxazolyl, and quinolinyl;
2.38 Compound 2.34, wherein said heteroaryl is indazolyl (e.g., 1,5- or 1,6-disubstituted-3-indazolyl);
2.39 Any Compounds 2.34-2.38, wherein each R⁴ moiety is independently selected from methyl, ethyl, propyl, isopropyl, n-butyl, sec-butyl, t-butyl, methoxy, ethoxy, hydroxy, amino (—NH₂), halo (e.g., fluoro, chloro, bromo or iodo), cyano, benzyl, benzyloxy, phenoxy, benzoyl, acetyl, pivaloyl, —C(O)OH, —C(O)NH₂, —C(O)NH(CH₃), and C(O)N(CH₃)(CH₃);
2.40 Compound 2.39, wherein each R⁴ moiety is independently selected from methyl, ethyl, propyl, isopropyl, n-butyl, sec-butyl, t-butyl, methoxy, ethoxy, hydroxy, halo (e.g., fluoro, chloro, bromo or iodo) and cyano;
2.41 Compound 2.39, wherein each R⁴ moiety is independently selected from methyl, hydroxy, amino (—NH₂), halo (e.g., fluoro, chloro, bromo or iodo) and cyano;
2.42 Compound 2.38, wherein said indazolyl is a 1-(C₁₋₃alkyl)-5-halo-3-indazolyl, or a 1-(C₁₋₃alkyl)-6-halo-3-indazolyl), optionally wherein said C₁₋₃alkyl is methyl and/or said halo is fluoro;
2.43 Compound 11, or any of 2.1-2.32, wherein L is —CH₂CH₂CH₂O-(aryl), wherein said aryl is phenyl or napthyl, and said aryl is monosubstituted with a hydroxy group;
2.44 Compound 2.43, wherein said aryl is phenyl monosubstituted with a hydroxy group (e.g., ortho, meta, or para substituted);
2.45 Compound II, or any of 2.1-2.32, wherein L is-CH₂CH₂CH₂O-(aryl), wherein said aryl is phenyl or napthyl, and said aryl is substituted with two or more R⁴ moieties;
2.46 Compound 2.45, wherein said aryl is phenyl;
2.47 Compound 2.45 or 2.46, wherein each R⁴ moiety is independently selected from C₁₋₆alkyl (e.g., methyl), C₁₋₆alkoxy (e.g., methoxy), halo (e.g., F), cyano, hydroxy, amino (—NH₂), C₁₋₆alkylaryl (e.g., benzyl), C₁₋₆alkoxyaryl (e.g., benzyloxy), aryloxy (e.g., phenoxy), —C(O)-aryl, —C(O)—C₁₋₆alkyl, —C(O)OH, —C(O)NH₂, —C(O)NH(C₁₋₆alkyl), and C(O)N(C₁₋₆alkyl)(C₁₋₆alkyl);
2.48 Compound 2.47, wherein each R⁴ moiety is independently selected from methyl, ethyl, propyl, isopropyl, n-butyl, sec-butyl, t-butyl, methoxy, ethoxy, hydroxy, amino (—NH₂), halo (e.g., fluoro, chloro, bromo or iodo), cyano, benzyl, benzyloxy, phenoxy, benzoyl, acetyl, pivaloyl, —C(O)OH, —C(O)NH₂, —C(O)NH(CH₃), and C(O)N(CH₃)(CH₃);
2.49 Compound 2.47, wherein each R⁴ moiety is independently selected from methyl, ethyl, propyl, isopropyl, n-butyl, sec-butyl, t-butyl, methoxy, ethoxy, hydroxy, halo (e.g., fluoro, chloro, bromo or iodo), and cyano;
2.50 Compound 2.47, wherein each R⁴ moiety is independently selected from methyl, ethyl, hydroxy, halo (e.g., fluoro, chloro, bromo or iodo), and cyano;
2.51 Any of Compounds 2.45-2.50, wherein there are two or three R⁴ moieties;
2.52 Compound 2.51, wherein one R⁴ moiety is halo (e.g., fluoro, chloro, bromo or iodo) and the other R⁴ moiety or moieties is/are selected from methyl, ethyl, propyl, isopropyl, n-butyl, sec-butyl, t-butyl, halo (e.g., fluoro, chloro, bromo or iodo) methoxy, ethoxy, hydroxy, and cyano;
2.53 Compound 2.51, wherein one R⁴ moiety is halo (e.g., fluoro, chloro, bromo or iodo) and the other R⁴ moiety or moieties is/are selected from methyl, ethyl, hydroxy, halo (e.g., fluoro, chloro, bromo or iodo), and cyano;
2.54 Compound 2.51, wherein there are three R⁴ moieties, and two are methyl and one is halo (e.g., fluoro, chloro, bromo or iodo);
2.55 Compound 2.51, wherein there are two R⁴ moieties, and one is methyl or ethyl and one is halo (e.g., fluoro, chloro, bromo or iodo) or cyano;
2.56 Compound 2.51, wherein there are three R⁴ moieties are each are independently selected from cyano, fluoro, chloro, bromo, and iodo;
2.57 Compound 2.51, wherein there are two R⁴ moieties are each are independently selected from cyano, fluoro, chloro, bromo, and iodo;
2.58 Compound 2.51, wherein there are two R⁴ moieties are each are fluoro, or one is fluoro and one is chloro, or one is fluoro and one is cyano;
2.59 Any of Compounds 2.51-2.58, wherein there are three R⁴ moieties and they are located at the two ortho positions and the para position of the phenyl ring (with respect to the carbon atom at which group L is attached);
2.60 Any of Compounds 2.51-2.58, wherein there are two R⁴ moieties and they are located at one ortho position and one para position of the phenyl ring (with respect to the carbon atom at which group L is attached);
2.61 Any of Compounds 2.51-2.58, wherein there are two R⁴ moieties and they are located at one ortho position and one meta position of the phenyl ring (with respect to the carbon atom at which group L is attached):
2.62 Compound II, or any of 2.1-2.32, wherein L is —(CH₂)n-C(O)-(heteroaryl), wherein n is an integer selected from 2, 3 and 4, and wherein said heteroaryl is a monocyclic 5-membered heteroaryl substituted with one to three R⁴ moieties;
2.63 Compound 2.62, wherein n is 2; 2.64 Compound 2.62, wherein n is 3; 2.65 Compound 2.62, wherein n is 4; 2.66 Any of Compounds 2.62-2.65, wherein said heteroaryl is selected from thiophenyl, furanyl, pyrrolyl, imidazolyl, oxazolyl, isoxazolyl, thiazolyl, triazolyl, and tetrazolyl;
2.67 Compound 2.66, wherein said heteroaryl is selected from thiophenyl and furanyl;
2.68 Compound 2.66, wherein said heteroaryl is thiophenyl (e.g., 5-substituted-2-thiophenyl or 4-substituted-3-thiophenyl);
2.69 Any of Compounds 2.62-2.68, wherein said heteroaryl is substituted with one R⁴ moiety;
2.70 Any of Compounds 2.62-2.68, wherein said heteroaryl is substituted with two R⁴ moieties;
2.71 Any of Compounds 2.62-2.68, wherein said heteroaryl is substituted with three R⁴ moieties;
2.72 Any of Compounds 2.62-2.71, wherein each R⁴ moiety is independently selected from C₁₋₆alkyl (e.g., methyl), C₁₋₆alkoxy (e.g., methoxy), halo (e.g., F), cyano, hydroxy, amino (—NH₂), C₁₋₆alkylaryl (e.g., benzyl), C₁₋₆alkoxyaryl (e.g., benzyloxy), aryloxy (e.g., phenoxy), —C(O)-aryl, —C(O)—C₁₋₆alkyl, —C(O)OH, —C(O)NH₂, —C(O)NH(C₁₋₆alkyl), and C(O)N(C₁₋₆alkyl)(C₁₋₆alkyl);
2.73 Compound 2.72, wherein each R⁴ moiety is independently selected from methyl, ethyl, propyl, isopropyl, n-butyl, sec-butyl, t-butyl, methoxy, ethoxy, hydroxy, amino (—NH₂), halo (e.g., fluoro, chloro, bromo or iodo), cyano, benzyl, benzyloxy, phenoxy, benzoyl, acetyl, pivaloyl, —C(O)OH, —C(O)NH₂, —C(O)NH(CH₃), and C(O)N(CH₃)(CH₃);
2.74 Compound 2.72, wherein each R⁴ moiety is independently selected from methyl, ethyl, propyl, isopropyl, n-butyl, sec-butyl, t-butyl, methoxy, ethoxy, hydroxy, halo (e.g., fluoro, chloro, bromo or iodo), and cyano;
2.75 Compound 2.72, wherein each R⁴ moiety is independently selected from methyl, hydroxy, halo (e.g., fluoro, chloro, bromo or iodo), and cyano;
2.76 Compound 2.72, wherein each R⁴ moiety is fluoro or chloro (e.g., chloro);
2.77 Any of Compounds 2.62-2.65, wherein n is 3, said heteroaryl is thiophenyl, and said thiophenyl is substituted with one or two R⁴ moieties, and each R⁴ moiety is independently selected from C₁₋₆alkyl (e.g., methyl), C₁₋₆alkoxy (e.g., methoxy), halo (e.g., F), cyano, hydroxy, amino (—NH₂), C₁₋₆alkylaryl (e.g., benzyl), C₁₋₆alkoxyaryl (e.g., benzyloxy), aryloxy (e.g., phenoxy), —C(O)-aryl, —C(O)—C₁₋₆alkyl, —C(O)OH, —C(O)NH₂, —C(O)NH(C₁₋₆alkyl), and C(O)N(C₁₋₆alkyl)(C₁₋₆alkyl);
2.78 Compound 2.77, wherein each R⁴ moiety is independently selected from methyl, ethyl, propyl, isopropyl, n-butyl, sec-butyl, t-butyl, methoxy, ethoxy, hydroxy, amino (—NH₂), halo (e.g., fluoro, chloro, bromo or iodo), cyano, benzyl, benzyloxy, phenoxy, benzoyl, acetyl, pivaloyl, —C(O)OH, —C(O)NH₂, —C(O)NH(CH₃), and C(O)N(CH₃)(CH₃);
2.79 Compound 2.77, wherein each R⁴ moiety is independently selected from methyl, ethyl, propyl, isopropyl, n-butyl, sec-butyl, t-butyl, methoxy, ethoxy, hydroxy, halo (e.g., fluoro, chloro, bromo or iodo), and cyano;
2.80 Compound 2.77, wherein each R⁴ moiety is independently selected from methyl, hydroxy, halo (e.g., fluoro, chloro, bromo or iodo) and cyano;
2.81 Compound 2.77, wherein each R⁴ moiety is fluoro or chloro (e.g., chloro);
2.82 Any of Compounds 2.77-2.81, wherein said thiophenyl is 5-substituted-2-thiophenyl or 4-substituted-3-thiophenyl;
2.83 Compound II, or any of 2.1-2.32, wherein L is —CH₂CH₂CH₂S(O)₂-(aryl), and wherein said aryl is phenyl or napthyl, and said aryl is substituted by one or two R⁴ moieties;
2.84 Compound 2.83, wherein said aryl is unsubstituted phenyl or unsubstituted napthyl;
2.85 Compound 2.83, wherein said aryl is phenyl or napthyl, and each R⁴ moiety is independently selected from C₁₋₆alkyl (e.g., methyl), C₁₋₆alkoxy (e.g., methoxy), halo (e.g., F), cyano, hydroxy, amino (—NH₂), C₁₋₆alkylaryl (e.g., benzyl), C₁₋₆alkoxyaryl (e.g., benzyloxy), aryloxy (e.g., phenoxy), —C(O)-aryl, —C(O)—C₁₋₆alkyl, —C(O)OH, —C(O)NH₂, —C(O)NH(C₁₋₆alkyl), and C(O)N(C₁₋₆alkyl)(C₁₋₆alkyl);
2.86 Compound 2.85, wherein each R⁴ moiety is independently selected from methyl, ethyl, propyl, isopropyl, n-butyl, sec-butyl, t-butyl, methoxy, ethoxy, hydroxy, amino (—NH₂), halo (e.g., fluoro, chloro, bromo or iodo), cyano, benzyl, benzyloxy, phenoxy, benzoyl, acetyl, pivaloyl, —C(O)OH, —C(O)NH₂, —C(O)NH(CH₃), and C(O)N(CH₃)(CH₃);
2.87 Compound 2.85, wherein each R⁴ moiety is independently selected from methyl, ethyl, propyl, isopropyl, n-butyl, sec-butyl, t-butyl, methoxy, ethoxy, hydroxy, halo (e.g., fluoro, chloro, bromo or iodo), and cyano;
2.88 Compound 2.85, wherein each R⁴ moiety is independently selected from methyl, hydroxy, halo (e.g., fluoro, chloro, bromo or iodo), and cyano;
2.89 Any of Compounds 2.85-2.88, wherein there are two or three R⁴ moieties;
2.90 Compound 2.89, wherein one R⁴ moiety is halo (e.g., fluoro, chloro, bromo or iodo) and the other R⁴ moiety or moieties is/are selected from methyl, ethyl, propyl, isopropyl, n-butyl, sec-butyl, t-butyl, halo (e.g., fluoro, chloro, bromo or iodo) methoxy, ethoxy, hydroxy, and cyano;
2.91 Compound 2.89, wherein one R⁴ moiety is halo (e.g., fluoro, chloro, bromo or iodo) and the other R⁴ moiety or moieties is/are selected from methyl, hydroxy, halo (e.g., fluoro, chloro, bromo or iodo), and cyano;
2.92 Compound 2.89, wherein there are one, two or three R⁴ moieties are each are independently selected from fluoro, chloro, bromo, and iodo;
2.93 Compound 2.89, wherein there is one R⁴ moieties selected from fluoro, chloro, bromo, and iodo;
2.94 Compound II, or any of 2.1-2.32, wherein L is CH₂CH₂CH₂O-(aryl), wherein said aryl is phenyl or napthyl, and said aryl is substituted with one or more R⁴ moieties, provided that R² and R³ are F;
2.95 Compound 2.94, wherein said aryl is unsubstituted phenyl or unsubstituted napthyl;
2.96 Compound 2.94, wherein said aryl is phenyl or napthyl, and each R⁴ moiety is independently selected from C₁₋₆alkyl (e.g., methyl), C₁₋₆alkoxy (e.g., methoxy), halo (e.g., F), cyano, hydroxy, amino (—NH₂), C₁₋₆alkylaryl (e.g., benzyl), C₁₋₆alkoxyaryl (e.g., benzyloxy), aryloxy (e.g., phenoxy), —C(O)-aryl, —C(O)—C₁₋₆alkyl, —C(O)OH, —C(O)NH₂, —C(O)NH(C₁₋₆alkyl), and C(O)N(C₁₋₆alkyl)(C₁₋₆alkyl);
2.97 Compound 2.96, wherein each R⁴ moiety is independently selected from methyl, ethyl, propyl, isopropyl, n-butyl, sec-butyl, t-butyl, methoxy, ethoxy, hydroxy, amino (—NH₂), halo (e.g., fluoro, chloro, bromo or iodo), cyano, benzyl, benzyloxy, phenoxy, benzoyl, acetyl, pivaloyl, —C(O)OH, —C(O)NH₂, —C(O)NH(CH₃), and C(O)N(CH₃)(CH₃);
2.98 Compound 2.96, wherein each R⁴ moiety is independently selected from methyl, ethyl, propyl, isopropyl, n-butyl, sec-butyl, t-butyl, methoxy, ethoxy, hydroxy, halo (e.g., fluoro, chloro, bromo or iodo), and cyano;
2.99 Compound 2.96, wherein each R⁴ moiety is independently selected from methyl, hydroxy, halo (e.g., fluoro, chloro, bromo or iodo), and cyano;
2.100 Any of Compounds 2.96-2.99, wherein there are two or three R⁴ moieties;
2.101 Compound 2.100, wherein one R⁴ moiety is halo (e.g., fluoro, chloro, bromo or iodo) and the other R⁴ moiety or moieties is/are selected from methyl, ethyl, propyl, isopropyl, n-butyl, sec-butyl, t-butyl, halo (e.g., fluoro, chloro, bromo or iodo) methoxy, ethoxy, hydroxy, and cyano;
2.102 Compound 2.100, wherein one R⁴ moiety is halo (e.g., fluoro, chloro, bromo or iodo) and the other R⁴ moiety or moieties is/are selected from methyl, hydroxy, halo (e.g., fluoro, chloro, bromo or iodo), and cyano;
2.103 Compound 2.100, wherein there are one, two or three R⁴ moieties are each are independently selected from cyano, fluoro, chloro, bromo, and iodo;
2.104 Compound 2.100, wherein there is one R⁴ moieties selected from fluoro, chloro, bromo, and iodo;
2.105 Compound II, or any of 2.1-2.104, wherein the compound is selected from the group consisting of:

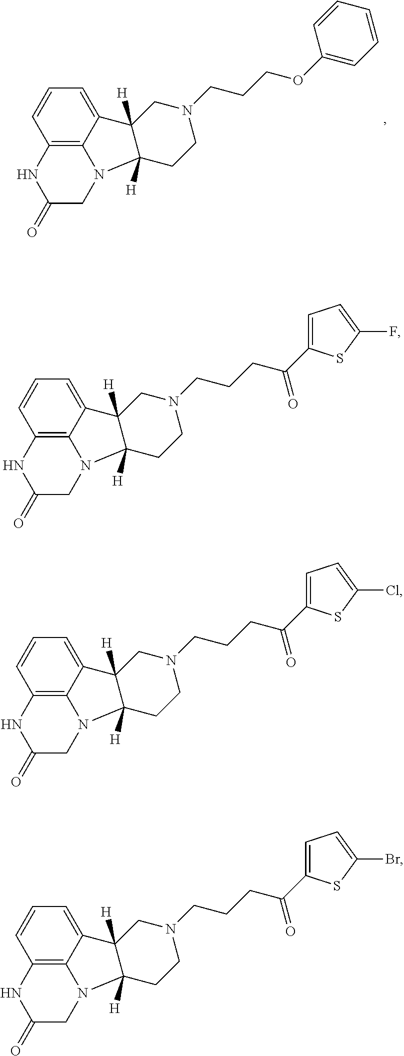 [SMILES: O=C1CN2c3c(cccc3[C@@H]3CN(CCCOc4ccccc4)CC[C@@H]32)N1 ; O=C1CN2c3c(cccc3[C@@H]3CN(CCCC(=O)c4ccc(F)s4)CC[C@@H]32)N1 ; O=C1CN2c3c(cccc3[C@@H]3CN(CCCC(=O)c4ccc(Cl)s4)CC[C@@H]32)N1 ; O=C1CN2c3c(cccc3[C@@H]3CN(CCCC(=O)c4ccc(Br)s4)CC[C@@H]32)N1]

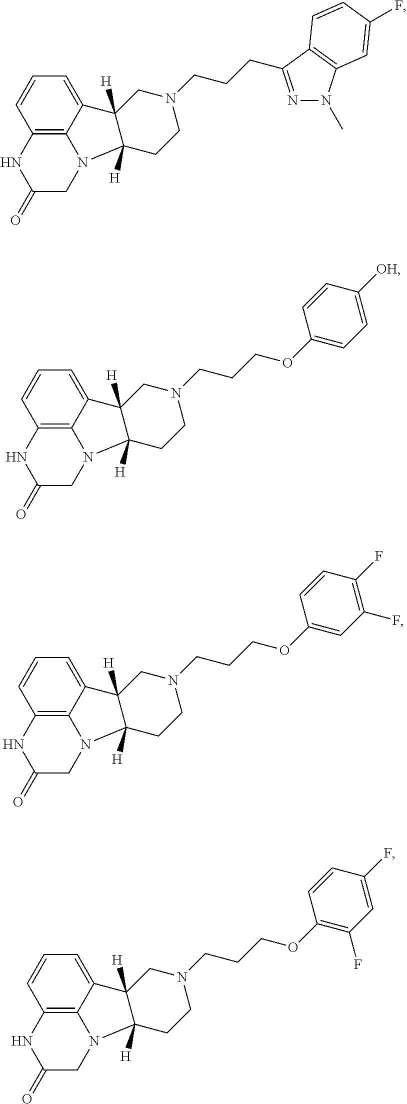 [SMILES: Cn1nc(CCCN2CC[C@H]3[C@@H](C2)c2cccc4c2N3CC(=O)N4)c2ccc(F)cc21 ; O=C1CN2c3c(cccc3[C@@H]3CN(CCCOc4ccc(O)cc4)CC[C@@H]32)N1 ; O=C1CN2c3c(cccc3[C@@H]3CN(CCCOc4ccc(F)c(F)c4)CC[C@@H]32)N1 ; O=C1CN2c3c(cccc3[C@@H]3CN(CCCOc4ccc(F)cc4F)CC[C@@H]32)N1]

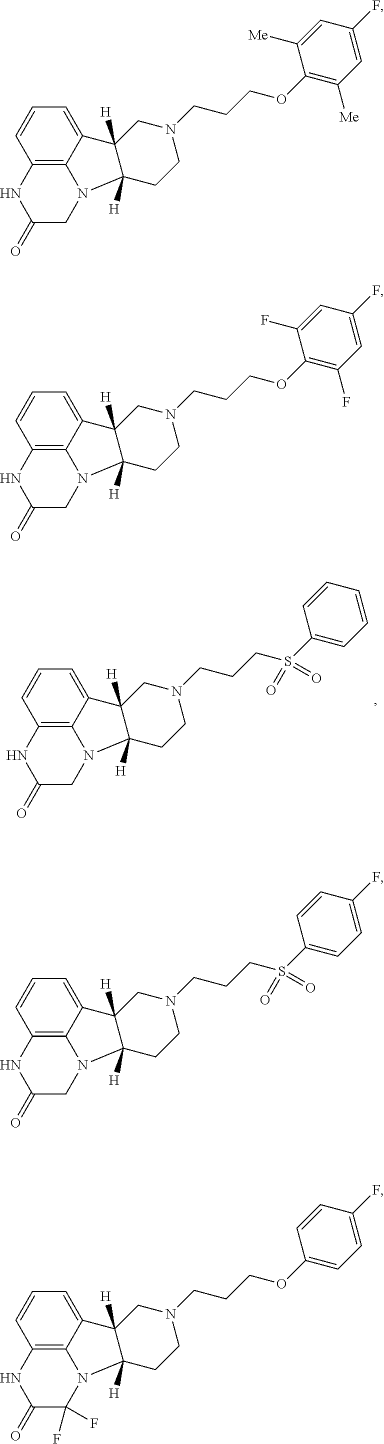 [SMILES: Cc1cc(F)cc(C)c1OCCCN1CC[C@H]2[C@@H](C1)c1cccc3c1N2CC(=O)N3 ; O=C1CN2c3c(cccc3[C@@H]3CN(CCCOc4c(F)cc(F)cc4F)CC[C@@H]32)N1 ; O=C1CN2c3c(cccc3[C@@H]3CN(CCCS(=O)(=O)c4ccccc4)CC[C@@H]32)N1 ; O=C1CN2c3c(cccc3[C@@H]3CN(CCCS(=O)(=O)c4ccc(F)cc4)CC[C@@H]32)N1 ; O=C1Nc2cccc3c2N([C@H]2CCN(CCCOc4ccc(F)cc4)C[C@@H]32)C1(F)F]

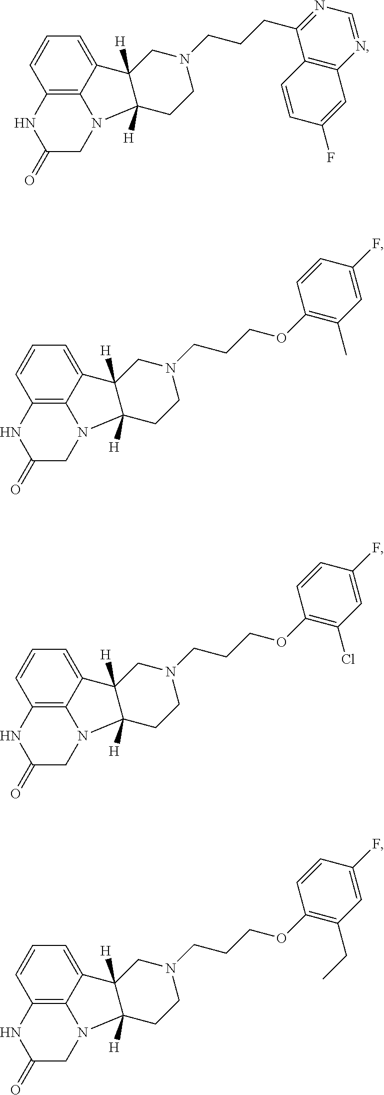 [SMILES: O=C1CN2c3c(cccc3[C@@H]3CN(CCCc4ncnc5cc(F)ccc45)CC[C@@H]32)N1 ; Cc1cc(F)ccc1OCCCN1CC[C@H]2[C@@H](C1)c1cccc3c1N2CC(=O)N3 ; O=C1CN2c3c(cccc3[C@@H]3CN(CCCOc4ccc(F)cc4Cl)CC[C@@H]32)N1 ; CCc1cc(F)ccc1OCCCN1CC[C@H]2[C@@H](C1)c1cccc3c1N2CC(=O)N3]

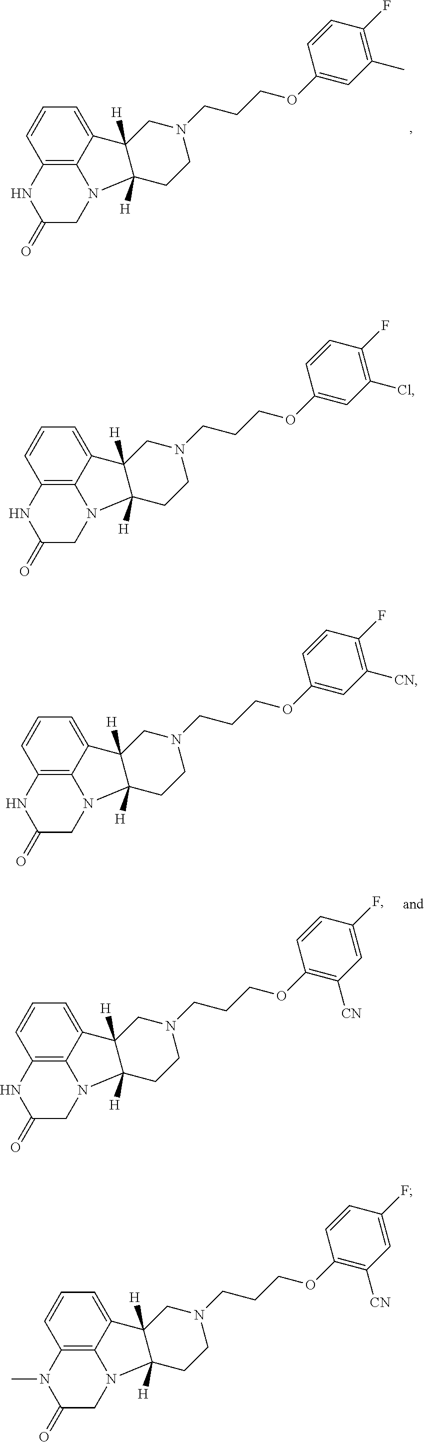

each independently in free or pharmaceutically acceptable salt or form;
2.106 Compound II, or any of 2.1-2.105, in free form;
2.107 Compound 11, or any of 2.1-2.105, in salt form, e.g., pharmaceutically acceptable salt form;
2.108 Compound II, or any of 2.1-2.105, wherein the compound is in acid addition salt form, for example, hydrochloric or toluenesulfonic acid addition salt form;
2.109 Compound II, or any of 2.1-2.106, in substantially pure diastereomeric form (i.e., substantially free from other diastereomers);
2.110 Compound II, or any of 2.1-2.109, having a diastereomeric excess of greater than 70%, preferably greater than 80%, more preferably greater than 90% and most preferably greater than 95%;
2.111 Compound II, or any of 2.1-2.110, in solid form, e.g., in crystal form;
2.112 Compound II, or any of 2.1-2.111, in isolated or purified form (e.g., in at least 90% pure form, or at least 95% or at least 98% or at least 99%).

In a third embodiment of the first aspect, the present disclosure relates to a compound (Compound III) of Formula III:

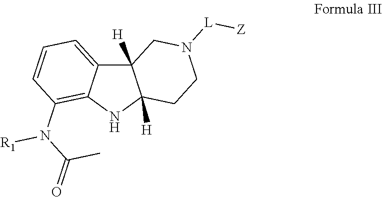

wherein:
R¹ is H, C₁₋₆alkyl, —C(O)—O—C(R^{a})(R^{b})(R^{c}), —C(O)—O—CH₂—O—C(R^{a})(R^{b})(R^{c}) or —C(R⁶)(R⁷)—O—C(O)—R⁸;
L is C₁₋₆alkylene (e.g., ethylene, propylene, or butylene), C₁₋₆alkoxy (e.g., propoxy or butoxy), C₂₋₃alkoxyC₁₋₃alkylene (e.g., CH₂CH₂OCH₂), C₁₋₆alkylamino or N—C₁₋₆alkyl C₁₋₆alkylamino (e.g., propylamino or N-methylpropylamino), C₁₋₆alkylthio (e.g., —CH₂CH₂CH₂S—), C₁₋₆alkylsulfonyl (e.g., —CH₂CH₂CH₂S(O)₂—), —C₁₋₆alkyl-C(O)— (e.g., 4-butanoyl), or —C₁₋₆alkyl-C(OH)— (e.g., 4-butanolyl), each of which is optionally substituted with one or more R⁴ moieties;
each R⁴ is independently selected from C₁₋₆alkyl (e.g., methyl), C₁₋₆alkoxy (e.g., methoxy), halo (e.g., F), cyano, hydroxy, amino (—NH₂), C₁₋₆alkylaryl (e.g., benzyl), C₁₋₆ alkoxyaryl (e.g., benzyloxy), aryloxy (e.g., phenoxy), —C(O)-aryl, —C(O)—C₁₋₆alkyl, —C(O)OH, —C(O)NH₂, —C(O)NH(C₁₋₆alkyl), and C(O)N(C₁₋₆alkyl)(C₁₋₆alkyl);
Z is selected from aryl (e.g., phenyl) and heteroaryl (e.g., thiophenyl, furanyl, pyridyl, indazolyl, benzimidazolyl, benzisoxazolyl), methyl or OH, each optionally substituted with one or more R⁴ moieties, optionally wherein Z is unsubstituted;
R⁸ is —C(R^{a})(R^{b})(R^{c}), —O—C(R^{a})(R^{b})(R^{c}), or —N(R^{d})(R^{e});
R^{a}, R^{b} and R^{c} are each independently selected from H and C₁₋₂₄alkyl;
R^{d} and R^{e} are each independently selected from H and C₁₋₂₄alkyl;
R⁶ and R⁷ are each independently selected from H, C₁₋₆alkyl, carboxy and C₁₋₆alkoxycarbonyl;
in free or salt form (e.g., pharmaceutically acceptable salt form), for example in an isolated or purified free or salt form (e.g., pharmaceutically acceptable salt form).

The present disclosure provides additional exemplary embodiments of the Compound of Formula III, in free or salt form (e.g., pharmaceutically acceptable salt form), for example in an isolated or purified free or salt form (e.g., pharmaceutically acceptable salt form), including:
3.1. Compound III, wherein R¹ is H;
3.2. Compound III, wherein R¹ is C₁₋₆alkyl, e.g., methyl;
3.3. Compound III, wherein R¹ is —C(O)—O—C(R^{a})(R^{b})(R^{e});
3.4. Compound 3.3, wherein R^{a} is H and R^{b} and R^{c} are each independently selected from C₁₋₂₄alkyl, e.g., C₁₋₂₀alkyl, C₅₋₂₀alkyl, C₉₋₁₈alkyl, C₁₀₋₁₆alkyl, or C₁₁alkyl, C₁₂alkyl, C₁₃alkyl, C₁₄alkyl, C₁₅alkyl or C₁₆alkyl;
3.5. Compound 3.3, wherein R^{a} and R^{b} are H and R^{c} is C₁₋₂₄alkyl, e.g., C₁₋₂₀alkyl, C₅₋₂₀alkyl, C₉₋₁₈alkyl, C₁₀₋₁₆alkyl, or C₁₁alkyl, C₁₂alkyl, C₁₃alkyl, C₁₄alkyl, C₁₅alkyl or C₁₆alkyl;
3.6. Compound 3.3, wherein R^{a}, R^{b} and R^{c} are each independently selected from C₁₋₂₄alkyl, e.g., C₁₋₂₀alkyl, C₅₋₂₀alkyl, C₉₋₁₈alkyl, C₁₀₋₁₆alkyl, or C₁₁alkyl, C₁₂alkyl, C₁₃alkyl, C₁₄alkyl, C₁₅alkyl or C₁₆alkyl;
3.7. Compound 3.3, wherein R^{a}, R^{b} and R^{c} are each H;
3.8. Compound 3.3, wherein R^{a} and R^{b} are H and R^{c} is C₁₀₋₁₄alkyl (e.g., R^{c} is CH₃(CH₂)₁₀ or CH₃(CH₂)₁₄);
3.9. Compound III, wherein R¹ is —C(O)—O—CH₂—O—C(R^{a})(R^{b})(R^{c}).
3.10. Compound 3.9, wherein R^{a} is H and R^{b} and R^{c} are each independently selected from C₁₋₂₄alkyl, e.g., C₁₋₂₀alkyl, C₅₋₂₀alkyl, C₉₋₁₈alkyl, C₁₀₋₁₆alkyl, or C₁₁alkyl, C₁₂alkyl, C₁₃alkyl, C₁₄alkyl, C₁₅alkyl or C₁₆alkyl;
3.11. Compound 3.9, wherein R^{a} and R^{b} are H and R^{c} is C₁₋₂₄alkyl, e.g., C₁₋₂₀alkyl, C₅₋₂₀alkyl, C₉₋₁₈alkyl, C₁₀₋₁₆alkyl, or C₁₁alkyl, C₁₂alkyl, C₁₃alkyl, C₁₄alkyl, C₁₅alkyl or C₁₆alkyl;
3.12. Compound 3.9, wherein R^{a}, R^{b} and R^{c} are each independently selected from C₁₋₂₄alkyl, e.g., C₁₋₂₀alkyl, C₅₋₂₀alkyl, C₉₋₁₈alkyl, C₁₀₋₁₆alkyl, or C₁₁alkyl, C₁₂alkyl, C₁₃alkyl, C₁₄alkyl, C₁₅alkyl or C₁₆alkyl;
3.13. Compound 3.9, wherein R^{a}, R^{b} and R^{c} are each H;
3.14. Compound III, wherein R¹ is —C(R⁶)(R⁷)—O—C(O)—R⁸, and R⁸ is —C(R^{a})(R^{b})(R^{c});
3.15. Compound III, wherein R¹ is —C(R⁶)(R⁷)—O—C(O)—R⁸, and R⁸ is —O—C(R^{a})(R^{b})(R^{c});
3.16. Compound 3.14 or 3.15, wherein R^{a} is H and R^{b} and R^{c} are each independently selected from C₁₋₂₄alkyl, e.g., C₁₋₂₀alkyl, C₅₋₂₀alkyl, C₉₋₁₈alkyl, C₁₀₋₁₆alkyl, or C₁₁alkyl, C₁₂alkyl, C₁₃alkyl, C₁₄alkyl, C₁₅alkyl or C₁₆alkyl;
3.17. Compound 3.14 or 3.15, wherein R^{a} and R^{b} are H and R^{c} is C₁₋₂₄alkyl, e.g., C₁₋₂₀alkyl, C₅₋₂₀alkyl, C₉₋₁₈alkyl, C₁₀₋₁₆alkyl, or C₁₁alkyl, C₁₂alkyl, C₁₃alkyl, C₁₄alkyl, C₁₅alkyl or C₁₆alkyl;
3.18. Compound 3.14 or 3.15, wherein R^{a}, R^{b} and R^{c} are each independently selected from C₁₋₂₄alkyl, e.g., C₁₋₂₀alkyl, C₅₋₂₀alkyl, C₉₋₁₈alkyl, C₁₀₋₁₆alkyl, or C₁₁alkyl, C₁₂alkyl, C₁₃alkyl, C₁₄alkyl, C₁₅alkyl or C₁₆alkyl;
3.19. Compound 3.14 or 3.15, wherein R^{a}, R^{b} and R^{c} are each H;
3.20. Any of compounds 3.14-3.19, wherein R⁶ is H, and R⁷ is C₁₋₃alkyl (e.g., R⁷ is methyl or isopropyl), and R⁸ is C₁₀₋₁₄alkyl (e.g., R⁸ is CH₃(CH₂)₁₀ or CH₃(CH₂)₁₄);
3.21. Compound III, wherein R¹ is —C(R⁶)(R⁷)—O—C(O)—R⁸, and R⁸ is —N(R^{d})(R^{e});
3.22. Compound 3.21, wherein R^{d} is H and R^{c} is independently selected from C₁₋₂₄alkyl, e.g., C₁₋₂₀alkyl, C₅₋₂₀alkyl, C₉₋₁₈alkyl, C₁₀₋₁₆alkyl, or C₁₁alkyl, C₁₂alkyl, C₁₃alkyl, C₁₄alkyl, C₁₅alkyl or C₁₆alkyl;
3.23. Compound 3.21, wherein R^{d} and R^{e} are each independently selected from C₁₋₂₄alkyl, e.g., C₁₋₂₀alkyl, C₅₋₂₀alkyl, C₉₋₁₈alkyl, C₁₀₋₁₆alkyl, or C₁₁alkyl, C₁₂alkyl, C₁₃alkyl, C₁₄alkyl, C₁₅alkyl or C₁₆alkyl;
3.24. Compound 3.21, wherein R^{d} and R^{e} are each H;
3.25. Any of Compounds 3.14-3.24, wherein R⁶ is H and R⁷ is H;
3.26. Any of Compounds 3.14-3.24, wherein R⁶ is C₁₋₆alkyl and R⁷ is C₁₋₆alkyl;
3.27. Any of Compounds 3.14-3.24, wherein R⁶ is H and R⁷ is C₁₋₆alkyl;
3.28. Any of Compounds 3.14-3.24, wherein R⁶ is H and R⁷ is carboxy;
3.29. Any of Compounds 3.14-3.24, wherein R⁶ is H and R⁷ is C₁₋₆alkoxycarbonyl, e.g., ethoxycarbonyl or methoxycarbonyl;
3.30. Compound III, or any of 3.1-3.29, wherein L is C₁₋₆alkylene (e.g., ethylene, propylene, or butylene), C₁₋₆alkoxy (e.g., propoxy), C₂₋₃alkoxyC₁₋₃alkylene (e.g., CH₂CH₂OCH₂) C₁₋₆alkylamino (e.g., propylamino or N-methylpropylamino), or C₁₋₆alkylthio (e.g., —CH₂CH₂CH₂S—), optionally substituted with one or more R⁴ moieties;
3.31. Compound 3.30, wherein L is unsubstituted C₁₋₆alkylene (e.g., ethylene, propylene, or butylene);
3.32. Compound 3.30, wherein L is C₁₋₆alkylene (e.g., ethylene, propylene, or butylene), substituted with one or more R⁴ moieties;
3.33. Compound 3.30, wherein L is unsubstituted C₁₋₆alkyoxy (e.g., propoxy or butoxy);
3.34. Compound 3.30, wherein L is C₁₋₆alkoxy (e.g., propoxy or butoxy), substituted with one or more R⁴ moieties;
3.35. Compound 3.30, wherein L is unsubstituted C₂₋₃alkoxyC₁₋₃alkylene (e.g., CH₂CH₂OCH₂);
3.36. Compound 3.30, wherein L is C₂₋₃alkoxyC₁₋₃alkylene (e.g., CH₂CH₂OCH₂), substituted with one or more R⁴ moieties;
3.37. Compound III, or any of 3.1-3.36, wherein L is —(CH₂)ₙ—X—, and wherein n is an integer selected from 2, 3 and 4, and X is selected from —O—, —S—, —NH—, —N(C₁₋₆alkyl)-, —CH₂—, —CH(OH)—, and —C(O)—;
3.38. Compound 3.37, wherein L is —(CH₂)ₙ—X—, and wherein n is an integer selected from 2, 3 and 4, and X is —O—;
3.39. Compound 3.37, wherein L is —(CH₂)ₙ—X—, and wherein n is an integer selected from 2, 3 and 4, and X is —C(O)—;
3.40. Compound 3.37, wherein L is —(CH₂)ₙ—X—, and wherein n is 3, and X is selected from —O—, —S—, —NH—, —N(C₁₋₆alkyl)- (e.g., —N(CH₃)—), —CH(OH), and —C(O)—;
3.41. Compound 3.37, wherein L is —(CH₂)ₙ—X—, and wherein n is 3, and X is selected from —O—, —S—, —CH(OH), and —C(O)—;
3.42. Compound 3.37, wherein L is —(CH₂)ₙ—X—, and wherein n is 3, and X is O;
3.43. Compound 3.37, wherein L is —(CH₂)ₙ—X—, and wherein n is 3, and X is S;
3.44. Compound 3.37, wherein L is —(CH₂)ₙ—X—, and wherein n is 3, and X is —CH(OH)—;
3.45. Compound 3.37, wherein L is —(CH₂)ₙ—X—, and wherein n is 3, and X is —C(O)—;
3.46. Compound III, or any of 3.1-3.45, wherein Z is aryl (e.g., phenyl), optionally substituted with one or more R⁴ moieties;
3.47. Compound 3.46, wherein Z is aryl (e.g., phenyl), substituted with one or more R⁴ moieties;
3.48. Compound 3.47, wherein Z is phenyl substituted with one, two, three or four R⁴ moieties;
3.49. Compound 3.47 or 3.48, wherein the one, two three or four R⁴ moieties are independently selected from methyl, methoxy, hydroxy, amino (—NH₂), halo (e.g., fluoro, chloro, bromo or iodo), cyano, benzyl, benzyloxy, phenoxy, benzoyl, acetyl, pivaloyl, —C(O)OH, —C(O)NH₂, —C(O)NH(CH₃), and C(O)N(CH₃)(CH₃);
3.50. Compound 3.47 or 3.48, wherein Z is phenyl substituted with one R⁴ moiety selected from methyl, methoxy, hydroxy, amino (—NH₂), halo (e.g., fluoro, chloro, bromo or iodo), cyano, benzyl, benzyloxy, phenoxy, benzoyl, acetyl, pivaloyl, —C(O)OH, —C(O)NH₂, —C(O)NH(CH₃), and C(O)N(CH₃)(CH₃);
3.51. Compound 3.47 or 3.48, wherein said Z is phenyl substituted with two R⁴ moieties each independently selected from methyl, halo (e.g., fluoro, chloro, bromo or iodo), and cyano;
3.52. Compound 111, or any of 3.1-3.45, wherein Z is heteroaryl (e.g., thiophenyl, furanyl, pyridyl, indazolyl, benzimidazolyl, benzisoxazolyl), optionally substituted with one or more R⁴ moieties;
3.53. Compound 3.52, wherein said heteroaryl is a monocyclic 5-membered or 6-membered heteroaryl (e.g., pyridyl, pyrimidyl, pyrazinyl, thiophenyl, pyrrolyl, thiophenyl, furanyl, imidazolyl, oxazolyl, isoxazolyl, thiazolyl);
3.54. Compound 3.53, wherein said heteroaryl is selected from pyridyl, pyrimidinyl and pyrazinyl;
3.55. Compound 3.53, wherein said heteroaryl is selected from thiophenyl, furanyl, pyrrolyl, imidazolyl, oxazolyl, isoxazolyl, thiazolyl, triazolyl, and tetrazolyl;
3.56. Compound 3.53, wherein said heteroaryl is selected from thiophenyl and furanyl;
3.57. Compound 3.53, wherein said heteroaryl is thiophenyl (e.g., 5-substituted-2-thiophenyl or 4-substituted-3-thiophenyl);
3.58. Compound 3.53, wherein said heteroaryl is a bicyclic 9-membered or 10-membered heteroaryl (e.g., indolyl, isoindolyl, benzofuranyl, benzothiophenyl, indazolyl, benzimidazolyl, benzoxazolyl, benzisoxazolyl, benzothiazolyl, quinolinyl, isoquinolinyl, quinoxalinyl, quinazolinyl, benzodioxolyl, 2-oxo-tetrahydroquinolinyl);
3.59. Compound 3.58, wherein said heteroaryl is selected from indazolyl, benzisoxazolyl, quinolinyl, quinazolinyl, benzodioxolyl, and 2-oxo-tetrahydroquinolinyl;
3.60. Compound 3.58, wherein said heteroaryl is selected from indazolyl, benzisoxazolyl, quinazolinyl, and quinolinyl;
3.61. Compound 3.60, wherein said heteroaryl is indazolyl (e.g., 5-substituted or 6-substituted-3-indazolyl, 1-substituted-3-indazolyl, or 1,5- or 1,6-disubstituted-3-indazolyl);
3.62. Compound 3.61, wherein said indazolyl is a 1-(C₁₋₃alkyl)-5-halo-3-indazolyl, or a 1-(C₁₋₃alkyl)-6-halo-3-indazolyl), optionally wherein said C₁₋₃alkyl is methyl and/or said halo is fluoro;
3.63. Any of Compounds 3.52-3.62, wherein said heteroaryl is substituted with one, two, three or four R⁴ moieties;
3.64. Compound 3.63, wherein the one, two three or four R⁴ moieties are independently selected from methyl, methoxy, ethoxy, hydroxy, amino (—NH₂), halo (e.g., fluoro, chloro, bromo or iodo), cyano, benzyl, benzyloxy, phenoxy, benzoyl, acetyl, pivaloyl, —C(O)OH, —C(O)NH₂, —C(O)NH(CH₃), and C(O)N(CH₃)(CH₃);
3.65. Compound 3.63, wherein said heteroaryl is substituted with one R⁴ moiety selected from methyl, methoxy, hydroxy, amino (—NH₂), halo (e.g., fluoro, chloro, bromo or iodo), cyano, benzyl, benzyloxy), phenoxy, benzoyl, acetyl, pivaloyl, —C(O)OH, —C(O)NH₂, —C(O)NH(CH₃), and C(O)N(CH₃)(CH₃);
3.66. Compound III, or any of 3.1-3.65, wherein the compound is selected from the group consisting of:

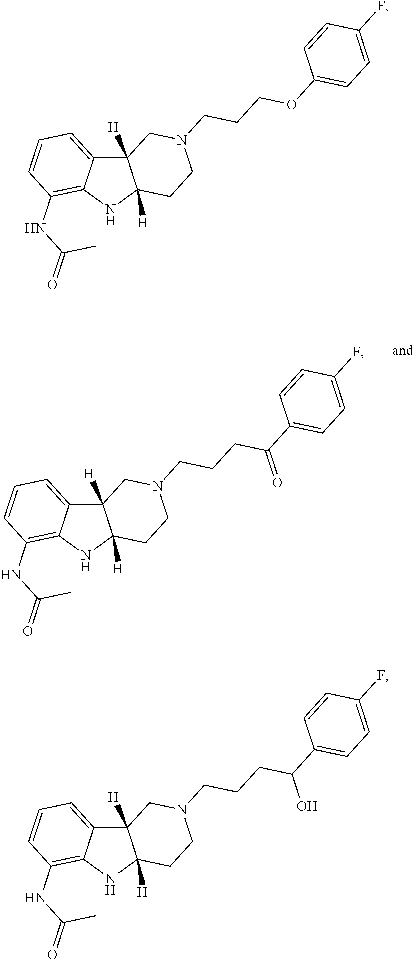 [SMILES: CC(=O)Nc1cccc2c1N[C@H]1CCN(CCCOc3ccc(F)cc3)C[C@@H]21 ; CC(=O)Nc1cccc2c1N[C@H]1CCN(CCCC(=O)c3ccc(F)cc3)C[C@@H]21 ; CC(=O)Nc1cccc2c1N[C@H]1CCN(CCCC(O)c3ccc(F)cc3)C[C@@H]21]

each independently in free or pharmaceutically acceptable salt or form;
3.67. Compound III, or any of 3.1-3.66, in free form;
3.68. Compound III, or any of 3.1-3.66, in salt form, e.g., pharmaceutically acceptable salt form;
3.69. Compound I, or any of 3.1-3.66, wherein the compound is in acid addition salt form, for example, hydrochloric or toluenesulfonic acid addition salt form;
3.70. Compound 111, or any of 3.1-3.69, in substantially pure diastereomeric form (i.e., substantially free from other diasteromers);
3.71. Compound III, or any of 3.1-3.69, having a diastereomeric excess of greater than 70%, preferably greater than 80%, more preferably greater than 90% and most preferably greater than 95%;
3.72. Compound III, or any of 3.1-3.71, in solid form, e.g., in crystal form;
3.73. Compound III, or any of 3.1-3.72, in isolated or purified form (e.g., in at least 90% pure form, or at least 95% or at least 98% or at least 99%).

As used hereinbelow, the “Compound of the Invention” refers to a Compound of Formula I or any of 1.1-1.75, or a Compound of Formula II, or any of 2.1-2.112, or a Compound of Formula III, or any of 3.1-3.73.

In a second aspect, the present disclosure provides a pharmaceutical composition (Pharmaceutical Composition 1) comprising Compound of the Invention, e.g., in admixture with a pharmaceutically acceptable diluent or carrier. In a particular embodiment, the Compound of the Invention is in pharmaceutically acceptable salt form. In some embodiments, the pharmaceutical composition is in the form of a tablet or capsule, e.g., for gastroenteric absorption (i.e., absorption through the stomach and/or large and small intestines). In some embodiments, the pharmaceutical composition is an oral transmucosal composition, e.g., an orally dissolving tablet, wafer, film, gel or spray. For example, the composition may be a rapidly-dissolving sublingual or buccal tablet, wafer, film, or gel. In some embodiments, the pharmaceutical composition is formulated for intranasal or intrapulmonary administration (e.g., as an aerosol, mist, or powder for inhalation). In some embodiments, the pharmaceutical composition is formulated for intravenous, intrathecal, intramuscular, subcutaneous or intraperitoneal injection. In particular, pharmaceutical compositions for intramuscular or subcutaneous injection may be in the form of long-acting injectable compositions or depot compositions, e.g., providing for sustained or delayed release of the Compound of the Invention into the blood stream and body tissues. Alternatively, particularly as formulated for intravenous, intrathecal, intraperitoneal, or subcutaneous injection, the composition may be an immediate-acting composition, e.g., providing immediate release into the body fluids of the majority or entirety of the dose.

In a further embodiment, the Pharmaceutical Compositions of the present disclosure, are for a sustained or delayed release formulation (Pharmaceutical Composition 1-A), e.g., a depot formulation. In some embodiments, the Compound of the Invention is provided, preferably in free or pharmaceutically acceptable salt form, in admixture with a pharmaceutically acceptable diluent or carrier, in the form of an injectable depot, which provides sustained or delayed release of the compound.

In a particular embodiment, the Pharmaceutical Composition 1-A comprises a Compound of the Invention, in free base or pharmaceutically acceptable salt form, optionally in crystal form, wherein the compound has been milled to, or the compound crystallized to, microparticle or nanoparticle size, e.g., particles or crystals having a volume-based particle size (e.g., diameter or Dv50) of 0.5 to 100 microns, for example, for example, 5-30 microns, 10-20 microns, 20-100 microns, 20-50 microns or 30-50 microns. Such particles or crystals may be combined with a suitable pharmaceutically acceptable diluent or carrier, for example water, to form a depot formulation for injection. For example, the depot formulation may be formulated for intramuscular or subcutaneous injection with a dosage of drug suitable for 4 to 6 weeks of treatment. In some embodiments, the particles or crystals have a surface area of 0.1 to 5 m²/g, for example, 0.5 to 3.3 m²/g or from 0.8 to 1.2 m²/g.

In another embodiment, the present disclosure provides a Pharmaceutical Composition 1-B, which is Pharmaceutical Composition 1, wherein Compound of the Invention, is in a polymeric matrix. In one embodiment, the Compound of the present disclosure is dispersed or dissolved within the polymeric matrix. In a further embodiment, the polymeric matrix comprises standard polymers used in depot formulations such as polymers selected from a polyester of a hydroxyfatty acid and derivatives thereof, or a polymer of an alkyl alpha-cyanoacrylate, a polyalkylene oxalate, a polyortho ester, a polycarbonate, a polyortho-carbonate, a polyamino acid, a hyaluronic acid ester, and mixtures thereof. In a further embodiment, the polymer is selected from a group consisting of polylactide, poly d,l-lactide, poly glycolide, PLGA 50:50, PLGA 85:15 and PLGA 90:10 polymer. In another embodiment, the polymer is selected form poly(glycolic acid), poly-D,L-lactic acid, poly-L-lactic acid, copolymers of the foregoing, poly(aliphatic carboxylic acids), copolyoxalates, polycaprolactone, polydioxanone, poly(ortho carbonates), poly(acetals), poly(lactic acid-caprolactone), polyorthoesters, poly(glycolic acid-caprolactone), polyanhydrides, and natural polymers including albumin, casein, and waxes, such as, glycerol mono- and distearate, and the like. In a preferred embodiment, the polymeric matrix comprises poly(d,l-lactide-co-glycolide)), such as PLGA having a 50:50 to 90:10 molar ratio. In some embodiments, the PLGA is admixed with a solvent carrier, e.g., an aqueous solvent, an aqueous emulsion, or a non-aqueous organic solvent (preferably a pharmaceutically acceptable organic solvent). Suitable organic solvents, depending on the amount used in the injection, may include propylene glycol, polyethylene glycol, ethanol, dimethyl sulfoxide, N-methyl-2-pyrrolidone, glycofurol, solketal, glycerol formate, acetone, tetrahydrofurfuryl alcohol, diglyme, dimethyl isosorbide, ethyl lactate, or mixtures thereof.

The Pharmaceutical Composition 1-B is particularly useful for sustained or delayed release, wherein the Compound of the present disclosure is released upon degradation of the polymeric matrix. These Compositions may be formulated for controlled- and/or sustained-release of the Compounds of the present disclosure (e.g., as a depot composition) over a period of up to 180 days, e.g., from about 14 to about 30 to about 180 days. For example, the polymeric matrix may degrade and release the Compounds of the present disclosure over a period of about 30, about 60 or about 90 days. In another example, the polymeric matrix may degrade and release the Compounds of the present disclosure over a period of about 120, or about 180 days.

In still another embodiment, the Pharmaceutical Composition 1 or 1-A or 1-B may be formulated for administration by injection, for example, as a sterile solution, such as a sterile aqueous solution or a sterile non-aqueous solution.

In another embodiment, the present disclosure provides a Pharmaceutical Composition (Pharmaceutical Composition 1-C) comprising a Compound of the Invention as hereinbefore described, in an osmotic controlled release oral delivery system (OROS), which is described in US 2001/0036472 and US 2009/0202631, the contents of each of which applications are incorporated by reference in their entirety. Therefore in one embodiment, the present disclosure provides a pharmaceutical composition or device comprising (a) a gelatin capsule containing a Compound of the Invention in free or pharmaceutically acceptable salt form, optionally in admixture with a pharmaceutically acceptable diluent or carrier; (b) a multilayer wall superposed on the gelatin capsule comprising, in outward order from the capsule: (i) a barrier layer, (ii) an expandable layer, and (iii) a semipermeable layer; and (c) and orifice formed or formable through the wall (Pharmaceutical Composition P.1).

In another embodiment, the invention provides a pharmaceutical composition comprising a gelatin capsule containing a liquid, the Compound of the Invention in free or pharmaceutically acceptable salt form, optionally in admixture with a pharmaceutically acceptable diluent or carrier, the gelatin capsule being surrounded by a composite wall comprising a barrier layer contacting the external surface of the gelatin capsule, an expandable layer contacting the barrier layer, a semi-permeable layer encompassing the expandable layer, and an exit orifice formed or formable in the wall (Pharmaceutical Composition P.2).

In still another embodiment, the invention provides a composition comprising a gelatin capsule containing a liquid, the Compound of the Invention in free or pharmaceutically acceptable salt form, optionally in admixture with a pharmaceutically acceptable diluent or carrier, the gelatin capsule being surrounded by a composite wall comprising a barrier layer contacting the external surface of the gelatin capsule, an expandable layer contacting the barrier layer, a semipermeable layer encompassing the expandable layer, and an exit orifice formed or formable in the wall, wherein the barrier layer forms a seal between the expandable layer and the environment at the exit orifice (Pharmaceutical Composition P.3).

In still another embodiment, the invention provides a composition comprising a gelatin capsule containing a liquid, the Compound of the Invention in free or pharmaceutically acceptable salt form, optionally in admixture with a pharmaceutically acceptable diluent or carrier, the gelatin capsule being surrounded by a barrier layer contacting the external surface of the gelatin capsule, an expandable layer contacting a portion of the barrier layer, a semi-permeable layer encompassing at least the expandable layer, and an exit orifice formed or formable in the dosage form extending from the external surface of the gelatin capsule to the environment of use (Pharmaceutical Composition P.4). The expandable layer may be formed in one or more discrete sections, such as for example, two sections located on opposing sides or ends of the gelatin capsule.

In a particular embodiment, the Compound of the Invention in the Osmotic-controlled Release Oral Delivery System (i.e., in Composition P.1-P.4) is in a liquid formulation, which formulation may be neat, liquid active agent, liquid active agent in a solution, suspension, emulsion or self-emulsifying composition or the like.

Further information on Osmotic-controlled Release Oral Delivery System composition including characteristics of the gelatin capsule, barrier layer, an expandable layer, a semi-permeable layer; and orifice may be found in US 2001/0036472, the contents of which are incorporated by reference in their entirety.

Other Osmotic-controlled Release Oral Delivery System for the Compound of the Invention or the Pharmaceutical Composition of the present disclosure may be found in US 2009/0202631, the contents of which are incorporated by reference in their entirety. Therefore, in another embodiment, the invention provides a composition or device comprising (a) two or more layers, said two or more layers comprising a first layer and a second layer, said first layer comprises the Compound of the Invention in free or pharmaceutically acceptable salt form, optionally in admixture with a pharmaceutically acceptable diluent or carrier, said second layer comprises a polymer; (b) an outer wall surrounding said two or more layers; and (c) an orifice in said outer wall (Pharmaceutical Composition P.5).

Pharmaceutical Composition P.5 preferably utilizes a semi-permeable membrane surrounding a three-layer-core: in these embodiments, the first layer is referred to as a first drug layer and contains low amounts of drug (e.g., the Compound of the Invention) and an osmotic agent such as salt, the middle layer referred to as the second drug layer contains higher amounts of drug, excipients and no salt; and the third layer referred to as the push layer contains osmotic agents and no drug (Pharmaceutical Composition P.6). At least one orifice is drilled through the membrane on the first drug layer end of the capsule-shaped tablet.

Pharmaceutical Composition P.5 or P.6 may comprise a membrane defining a compartment, the membrane surrounding an inner protective subcoat, at least one exit orifice formed or formable therein and at least a portion of the membrane being semi-permeable; an expandable layer located within the compartment remote from the exit orifice and in fluid communication with the semi-permeable portion of the membrane; a first drug layer located adjacent the exit orifice; and a second drug layer located within the compartment between the first drug layer and the expandable layer, the drug layers comprising the Compound of the Invention in free or pharmaceutically acceptable salt thereof (Pharmaceutical Composition P.7). Depending upon the relative viscosity of the first drug layer and second drug layer, different release profiles are obtained. It is imperative to identify the optimum viscosity for each layer. In the present invention, viscosity is modulated by addition of salt, sodium chloride. The delivery profile from the core is dependent on the weight, formulation and thickness of each of the drug layers.

In a particular embodiment, the invention provides Pharmaceutical Composition P.7 wherein the first drug layer comprises salt and the second drug layer contains no salt. Pharmaceutical Composition P.5-P.7 may optionally comprise a flow-promoting layer between the membrane and the drug layers.

Pharmaceutical Compositions P.1-P.7 will generally be referred to as Osmotic-controlled Release Oral Delivery System Composition.

In a third aspect, the invention provides a method (Method 1) for the treatment or prophylaxis of a central nervous system disorder, or more than one central nervous system disorder, the method comprising administering to a patient in need thereof an effective amount of a Compound of the Invention or a pharmaceutical composition comprising an effective amount of a Compound of the Invention, or a pharmaceutical composition comprising a Compound of the Invention, e.g., Pharmaceutical Composition 1, 1-A, 1-B, 1-C, or any of P.1-P.7. In particular embodiments, Method 1 comprises administering:
1.1 A Compound of the Invention, in free form;
1.2 A Compound of the Invention, in pharmaceutically acceptable salt form;
1.3 A Compound of the Invention, in acid addition salt form;
1.4 Pharmaceutical Composition 1;
1.5 Any of Pharmaceutical Compositions 1-A, 1-B or 1-C;
1.6 Any of Pharmaceutical Composition P.1 to P.7; or
1.7 Any Osmotic-controlled Release Oral Delivery System Composition as hereinbefore described.

Substance-use disorders and substance-induced disorders are the two categories of substance-related disorders defined by the Fifth Edition of the DSM (the Diagnostic and Statistical Manual of Mental Disorders, DSM-5). A substance-use disorder is a pattern of symptoms resulting from use of a substance which the individual continues to take, despite experiencing problems as a result. A substance-induced disorder is a disorder induced by use if the substance. Substance-induced disorders include intoxication, withdrawal, substance induced mental disorders, including substance induced psychosis, substance induced bipolar and related disorders, substance induced depressive disorders, substance induced anxiety disorders, substance induced obsessive-compulsive and related disorders, substance induced sleep disorders, substance induced sexual dysfunctions, substance induced delirium and substance induced neurocognitive disorders.

The DSM-5 includes criteria for classifying a substance use disorder as mild, moderate or severe. In some embodiments of the methods disclosed herein, the substance use disorder is selected from a mild substance use disorder, a moderate substance use disorder or a severe substance use disorder. In some embodiments, the substance use disorder is a mild substance use disorder. In some embodiments, the substance use disorder is a moderate substance use disorder. In some embodiments, the substance use disorder is a severe substance use disorder.

Anxiety and depression are highly prevalent co-morbid disorders in patients undergoing treatment of substance use or substance abuse. A common treatment for substance abuse disorder is the combination of the partial opioid agonist buprenorphine with the opioid antagonist naloxone, but neither of these drugs has any significant effect on anxiety or depression, thus leading to the common result that a third drug, such as a benzodiazepine-class anxiolytic agent or an SSRI anti-depressant, must also be prescribed. This makes treatment regimens and patient compliance more difficult. In contrast, the Compounds of the present disclosure provide opioid antagonism along with serotonin antagonism and dopamine modulation. This may result in significant enhancement of treatment of patients with substance use or abuse disorder concomitant with anxiety and/or depression.

The Compounds of the Invention may have anxiolytic properties ameliorating the need for treatment of a patient with an anxiolytic agent where said patients suffers from co-morbid anxiety. Thus, in some embodiments, the present disclosure provides a method for the treatment of substance addiction, substance use disorders and/or substance-induced disorders, or a substance abuse disorder, for example, in a patient suffering from symptoms of anxiety or who is diagnosed with anxiety as a co-morbid disorder, or as a residual disorder, wherein the method does not comprise the further administration of an anxiolytic agent, such as a benzodiazepine. Benzodiazepines are GABA-modulating compounds.

The Compounds of the Invention may be particularly effective and useful for the treatment of pain, wherein the patient suffers from a gastrointestinal disorder and/or a pulmonary disorder. Traditional opioid analgesics suffer from two dominant side effects: gastrointestinal disturbances (including nausea, vomiting and constipation) and respiratory depression. 90 to 95% of patients taking opioids for long-term pain treatment develop serious constipation, requiring the long-term use of laxatives and/or enemas. The stronger opioids such as morphine, oxycodone and hydromorphone produce more severe constipation than other opioids. Respiratory depression is the most serious adverse effect of opioid treatment as it creates a risk of death, especially when patients combine (intentionally or inadvertently) prescribed opioid analgesics with other licit or illicit respiratory depressants (including alcohol). Patients in need of pain treatment, especially chronic pain treatment, are therefore at particular risk of adverse effects if they suffer from a pre-existing gastrointestinal or pulmonary disorder. Unlike traditional opioid analgesics, the compounds of the present invention provide analgesic relief without significant adverse gastrointestinal effects and without significant respiratory depression. Therefore, such compounds would provide improved safety and efficacy for patients in need of pain treatment having these preexisting GI and pulmonary disorders. In further embodiments, a compound of the present invention may be combined with a traditional opioid agent to provide improved pain control with a dose-sparing effect as to the traditional opioid agent (and concomitantly reduced risk of adverse effects).

In some embodiments, the pain is caused by post-herpetic neuralgia. Postherpetic neuralgia (PHN) is neuropathic pain which occurs due to damage to a peripheral nerve caused by the reactivation of the varicella zoster virus.

In some embodiments, the pain is caused by fibromyalgia, e.g., the pain is a symptom of fibromyalgia. Fibromyalgia is a complex syndrome of uncertain cause or origin. It is classified as a disorder of pain processing, and in particular, the processing of pain signals within the central nervous system. As such, it is like a central neuropathic pain syndrome, and it is often considered an example of “central sensitization.” Fibromyalgia is marked by chronic, widespread pain, often including allodynia. In the United States, only pregabalin and duloxetine have been approved for managing fibromyalgia, and existing analgesics have generally been ineffective.

Patients who suffer from a neuropathy who might otherwise be treated with an opioid analgesic, or other drugs associated with high risk of abuse, would be contra-indicated for such treatment if they suffer from a substance-use disorder or substance abuse disorder, or have had prior instances of opioid addiction, opioid withdrawal, or opioid overdose, or prior instances of substance abuse or alcohol abuse. Therefore, especially in such patients, there is a need for alternative, non-addictive treatment methods, such as the methods described herein.

In some embodiments of the methods described hereinbelow, the Pharmaceutical Composition comprising a Compound of the Invention may be administered for controlled- and/or sustained-release of the Compounds of the Invention over a period of from about 14 days, about 30 to about 180 days, preferably over the period of about 30, about 60 or about 90 days. Controlled- and/or sustained-release is particularly useful for circumventing premature discontinuation of therapy, particularly for antipsychotic drug therapy where non-compliance or non-adherence to medication regimes is a common occurrence.

In some embodiments of the methods described hereinbelow, the Pharmaceutical Composition comprising a Compound of the Invention may be a Depot Composition of the present disclosure which is administered for controlled- and/or sustained-release of the Compounds of the Invention over a period of time.

In a further embodiment of the third aspect, the present disclosure provides further embodiments of Method 1 as follows:
1.8 Method 1 or any of Methods 1.1-1.7, wherein the central nervous system disorder is a disorder involving serotonin 5-HT_{2A} receptor, dopamine D1 receptor, and/or D2 receptor systems, and/or the serotonin reuptake transporter (SERT) pathways, and/or the mu-opioid receptor pathway;
1.9 Method 1 or any of Methods 1.1-1.8, wherein the central nervous system disorder is a disorder selected from a group consisting of obesity, anxiety (including general anxiety, social anxiety, and panic disorders), depression (for example refractory depression and MDD), psychosis (including psychosis associated with dementia, such as hallucinations in advanced Parkinson's disease or paranoid delusions), schizophrenia, sleep disorders (particularly sleep disorders associated with schizophrenia and other psychiatric and neurological diseases), sexual disorders, migraine, pain and conditions associated with pain, including cephalic pain, idiopathic pain, chronic pain (such as moderate to moderately severe chronic pain, for example in patients requiring 24 hour extend treatment for other ailments), neuropathic pain, dental pain, fibromyalgia, chronic fatigue, agoraphobia, social phobias, agitation in dementia (e.g., agitation in Alzheimer's disease), agitation in autism and related autistic disorders, gastrointestinal disorders such as dysfunction of the gastrointestinal tract motility, and dementia, for example dementia of Alzheimer's disease or of Parkinson's disease; mood disorders; drug dependencies, for example, opioid dependency and/or alcohol dependency, and withdrawal from drug or alcohol dependency (e.g., opioid dependency); opioid overdose; co-morbidities associated with drug dependencies, such as depression, anxiety and psychosis; binge eating disorder; and obsessive-compulsive disorder (OCD), obsessive-compulsive personality disorder (OCPD) and related disorders; or opioid use disorder (OUD), or any combination thereof;
1.10 Method 1 or any of Methods 1.1-1.8, wherein the central nervous system disorder is a disorder selected from the following: (i) psychosis, e.g., schizophrenia, in a patient suffering from depression; (2) depression in a patient suffering from psychosis, e.g., schizophrenia; (3) mood disorders associated with psychosis and/or drug dependencies, e.g., schizophrenia or Parkinson's disease; (4) sleep disorders associated with psychosis, e.g., schizophrenia or Parkinson's disease; and (5) substance addiction, substance use disorders and/or substance-induced disorders, optionally wherein the patient suffers from residual symptoms of anxiety or anxiety disorder; and optionally wherein the depression is treatment-resistant depression;
1.11 Method 1 or any of Methods 1.1-1.8, wherein the central nervous system disorder is psychosis, e.g., schizophrenia, and said patient is a patient suffering from depression;
1.12 Method 1 or any of Methods 1.1-1.8, wherein the central nervous system disorder is depression, and said patient is a patient suffering from psychosis, e.g., schizophrenia, or Parkinson's disease;
1.13 Method 1 or any of 1.1-1.8, wherein said disorder is a drug dependency disorder, optionally in conjunction with any preceding disorders, for example, wherein said disorder is an opioid dependency, cocaine dependency, amphetamine dependency, alcohol dependency, or withdrawal from any drug or alcohol dependency (e.g., withdrawal from opioid, cocaine, or amphetamine dependency), or wherein said disorder is an opioid use disorder or opioid overdose; or wherein the method is a method for the treatment or prevention of opioid addiction relapse (e.g., for detoxification and maintenance treatment of opioid addiction or prevention of relapse to opioid addiction);
1.14 Method 1.13, wherein said patient also suffers from a co-morbidity, such as anxiety, depression or psychosis, or residual symptoms of anxiety or anxiety disorder and/or altered mood (e.g., depression); further optionally wherein the patient suffers from an opioid overdose;
1.15 Method 1 or any of Methods 1.1-1.8, wherein the central nervous system disorder is a disorder selected from obsessive-compulsive disorder (OCD), obsessive-compulsive personality disorder (OCPD), general anxiety disorder, social anxiety disorder, panic disorder, agoraphobia, compulsive gambling disorder, compulsive eating disorder, body dysmorphic disorder, hypochondriasis, pathological grooming disorder, kleptomania, pyromania, attention deficit-hyperactivity disorder (ADHD), attention deficit disorder (ADD), impulse control disorder, and related disorders, and combination thereof;
1.16 Method 1 or any one Method 1.1-1.8, wherein the central nervous system disorder is selected from obsessive-compulsive disorder (OCD), obsessive-compulsive personality disorder (OCPD), social anxiety disorder, panic disorder, agoraphobia, compulsive gambling disorder, compulsive eating disorder, body dysmorphic disorder and impulse control disorder;
1.17 Method 1 or any one of Method 1.1-1.8, wherein the central nervous system disorder is obsessive-compulsive disorder (OCD) or obsessive-compulsive personality disorder (OCPD);
1.18 Method 1 or any of Method 1.1-1.8, wherein the central nervous system disorder is a pain disorder, e.g., a condition associated with pain, such as cephalic pain, idiopathic pain, neuropathic pain, chronic pain (e.g., moderate to moderately severe chronic pain, for example, in patients requiring 24-hour extended treatment for other ailments), fibromyalgia, dental pain, traumatic pain, or chronic fatigue;
1.19 Method 1, or any of Methods 1.1-1.8, wherein the central nervous system disorder is opioid use disorder, opioid withdrawal or opioid dependency, and/or wherein the method provides relief from withdrawal-induced symptoms (e.g., gastrointestinal symptoms such as diarrhea, anxiety, depression, pain, sleep disturbances, or any combination thereof);
1.20 Method 1, or any of Methods 1.1-1.8, wherein the central nervous system disorder is chronic pain and/or neuropathic pain;
1.21 Method 1.20, wherein the pain is caused by a peripheral neuropathy (e.g., a mononeuropathy, a plexopathy, a radiculopathy, or a polyneuropathy) or is caused by a central neuropathy (e.g., deafferentation pain or sympathetically maintained pain, such as complex regional pain syndrome (CRPS));
1.22 Any of Methods 1.20-1.21, wherein the pain is a chronic pain.
1.23 Any of Methods 1.20-1.21, wherein the pain is a neuropathic pain.
1.24 Method 1.22 or 1.23, wherein the pain is a chronic neuropathic pain.
1.25 Any of Methods 1.20-1.24, wherein the pain is caused by a mononeuropathy (e.g., single mononeuropathy), such as a focal mononeuropathy, a pressure mononeuropathy, or an entrapment mononeuropathy (e.g., carpal tunnel syndrome);
1.26 Any of Methods 1.20-1.24, wherein the pain is caused by a radiculopathy, e.g., caused by a herniated spinal disk, or caused by diabetic ischemia;
1.27 Any of Methods 1.20-1.24, wherein the pain is caused by a plexopathy, such as, a plexopathy caused by nerve compression, e.g., nerve compression by a neuroma, tumor, or herniated disk;
1.28 Any of Methods 1.20-1.24, wherein the pain is caused by a multiple mononeuropathy or a polyneuropathy, e.g., diabetic polyneuropathy;
1.29 Any of Methods 1.20-1.24, wherein the pain is caused by a central neuropathic pain syndrome, such as deafferentation pain or complex regional pain syndrome (CRPS), or by fibromyalgia;
1.30 Any of Methods 1.20-1.24, wherein the pain is caused by postherpetic neuralgia (PHN) or by fibromyalgia;
1.31 Any of Methods 1.20-1.24, wherein the pain is caused by drug-induced neurotoxicity (e.g., by doxorubicin, etoposide, gemcitabine, ifosfamide, interferon alfa, platinum chemotherapeutics (e.g., cisplatin, carboplatin, oxaliplatin, nedaplatin, triplatin, phenanthriplatin, picoplatin, satraplatin), or vinca alkaloids (e.g., vinblastine, vincristine, vindesine, vinorelbine, or vinpocetin), or anti-retroviral nucleosides (e.g., didanosine, stavudine, zalcitabine));
1.32 Any of Methods 1.20-1.31, wherein the neuropathy is an axonal neuropathy (i.e., an axonopathy);
1.33 Any of Methods 1.20-1.32, wherein the patient has fibromyalgia, diabetes, human immunodeficiency virus (HIV) infection or acquired immune deficiency syndrome (AIDS), or cancer;
1.34 Any of Methods 1.20-1.32, wherein the patient is undergoing concurrent treatment or has had past treatment with an anti-retroviral nucleoside, a platinum-based anti-neoplastic, or a vinca alkaloid anti-neoplastic:
1.35 Any of Methods 1.20-1.34, wherein the pain is associated with allodynia and/or hyperalgesia;
1.36 Any of Methods 1.20-1.35, wherein the patient also suffers from anxiety (including general anxiety, social anxiety, and panic disorders), depression (for example refractory depression and MDD), psychosis (including psychosis associated with dementia, such as hallucinations in advanced Parkinson's disease or paranoid delusions), schizophrenia, migraine, substance abuse disorder, substance use disorder, opiate use disorder, or other drug dependencies, for example, stimulant dependency and/or alcohol dependency.
1.37 Method 1 or any of Method 1.1-1.8, wherein the central nervous system disorder is a sleep disorder;
1.38 Method 1.37, wherein the sleep disorder is selected from sleep maintenance insomnia, frequent awakenings, and waking up feeling unrefreshed;
1.39 Method 1.37, wherein the sleep disorder is in a patient suffering from or at risk of dyskinesia, e.g., a patient receiving dopaminergic medications, e.g., selected from levodopa and levodopa adjuncts (carbidopa, COMT inhibitors, MAO-B inhibitors), dopamine agonists, and anticholinergics, e.g., receiving levodopa;
1.40 Any of Methods 1.37-1.39, wherein the patient also suffers from Parkinson's disease.
1.41 Method 1 or any of Methods 1.1-1.8, wherein said disorder is a sleep disorder and said patient is suffering from depression;
1.42 Method 1 or any of Methods 1.1-1.8, wherein said disorder is a sleep disorder and said patient is suffering from psychosis, e.g., schizophrenia;
1.43 Method 1 or any of Methods 1.1-1.8, wherein said disorder is sleep disorder and said patient is suffering from Parkinson's disease;
1.44 Method 1 or any of Methods 1.1-1.8, wherein said disorder is sleep disorder and said patient is suffering from depression and psychosis, e.g., schizophrenia, and/or Parkinson's disease;
1.45 Method 1 or any of Methods 1.1-1.8, wherein the disorder is dyskinesia, e.g., in a patient receiving dopaminergic medications, e.g., medications selected from levodopa and levodopa adjuncts (carbidopa, COMT inhibitors, MAO-B inhibitors), dopamine agonists, and anticholinergics, e.g., levodopa;
1.46 Method 1, or any of 1.1-1.46, wherein the patients suffers from any combination of the disorders recited in Methods 1.1-1.45; 1.47 Method 1, or any of 1.1-1.46, wherein the method is a method for the treatment or prophylaxis of any combination of the disorders recited in Methods 1.1-1.45; 1.48 Method 1 or any of 1.1-1.47, wherein the patient suffers from a pre-existing or co-morbid gastrointestinal disorder and/or pulmonary disorder;
1.49 Method 1.48, wherein the pre-existing or co-morbid disorder is selected from the group consisting of irritable bowel syndrome, pelvic floor disorder, diverticulitis, inflammatory bowel disease, colon or colorectal cancer, celiac disease, non-celiac gluten sensitivity, asthma, chronic obstructive pulmonary disease (COPD), dyspnea, pneumonia, congestive heart failure, interstitial lung disease, pneumothorax, bronchitis, pulmonary embolism, and traumatic chest injury (e.g., broken sternum or ribs, bruised intercostal muscles);
1.50 Any of Methods 1.1-1.49, wherein the patient has been diagnosed with a substance use disorder or a substance abuse disorder, such as opioid use disorder (OUD);
1.51 Any of Methods 1.1-1.50, wherein said patient has a history of prior substance use or substance abuse with an opiate or opioid drug, e.g., morphine, codeine, thebaine, oripavine, morphine dipropionate, morphine dinicotinate, dihydrocodeine, buprenorphine, etorphine, hydrocodone, hydromorphone, oxycodone, oxymorphone, fentanyl, alpha-methylfentanyl, alfentanyl, trefantinil, brifentanil, remifentanil, ocfentanil, sufentanil, carfentanyl, meperidine, prodine, promedol, propoxyphene, dextropropoxyphene, methadone, diphenoxylate, dezocine, pentazocine, phenazocine, butorphanol, nalbuphine, levorphanol, levomethorphan, tramadol, tapentadol, and anileridine, or any combinations thereof;
1.52 Method 1 or any of 1.1-1.51, wherein said patient is or has been diagnosed with an opiate dependency, cocaine dependency, amphetamine dependency, and/or alcohol dependency, or suffers from withdrawal from drug or alcohol dependency (e.g. opiate, cocaine, or amphetamine dependency);
1.53 Method 1 or any of 1.1-1.52, wherein said patient has previously suffered from an opiate or opioid overdose;
1.54 Method 1 or any of 1.1-1.53, wherein the patient is not responsive to or cannot tolerate the side effects of non-narcotic analgesics and/or opiate and opioid drugs, or wherein the use of opiate or opioid drugs are contraindicated in said patient, for example, due to prior substance abuse or a high potential for substance abuse, such as opiate and opioid drugs including, e.g., morphine, codeine, thebaine, oripavine, morphine dipropionate, morphine dinicotinate, dihydrocodeine, buprenorphine, etorphine, hydrocodone, hydromorphone, oxycodone, oxymorphone, fentanyl, alpha-methylfentanyl, alfentanyl, trefantinil, brifentanil, remifentanil, ocfentanil, sufentanil, carfentanyl, meperidine, prodine, promedol, propoxyphene, dextropropoxyphene, methadone, diphenoxylate, dezocine, pentazocine, phenazocine, butorphanol, nalbuphine, levorphanol, levomethorphan, tramadol, tapentadol, and anileridine, or any combinations thereof;
1.55 Method 1 or any of Methods 1.1-1.54, wherein said patient is unable to tolerate the side effects of conventional antipsychotic drugs, e.g., chlorpromazine, haloperidol, droperidol, fluphenazine, loxapine, mesoridazine molindone, perphenazine, pimozide, prochlorperazine promazine, thioridazine, thiothixene, trifluoperazine, brexpiprazole, cariprazine, asenapine, lurasidone, clozapine, aripiprazole, olanzapine, quetiapine, risperidone and ziprasidone;
1.56 Method 1 or any of Methods 1.1-1.55, wherein said patient is unable to tolerate the side effects of non-narcotic analgesics and/or opioid and opioid drugs, or wherein the use of opioid drugs are contraindicated in said patient, for example, due to prior substance abuse or a high potential for substance abuse, such as opioid and opioid drugs including, e.g., morphine, codeine, thebaine, oripavine, morphine dipropionate, morphine dinicotinate, dihydrocodeine, buprenorphine, etorphine, hydrocodone, hydromorphone, oxycodone, oxymorphone, fentanyl, alpha-methylfentanyl, alfentanyl, trefantinil, brifentanil, remifentanil, ocfentanil, sufentanil, carfentanyl, meperidine, prodine, promedol, propoxyphene, dextropropoxyphene, methadone, diphenoxylate, dezocine, pentazocine, phenazocine, butorphanol, nalbuphine, levorphanol, levomethorphan, tramadol, tapentadol, and anileridine, or any combinations thereof;
1.57 Method 1 or any of Methods 1.1-1.56, wherein said patient is not responsive to or cannot tolerate the side effects from, treatment with selective serotonin reuptake inhibitors (SSRIs), such as citalopram, escitalopram, fluoxetine, fluvoxamine, paroxetine, and sertraline;
1.58 Method 1 or any of Methods 1.1-1.57, wherein said patient is not responsive to or cannot tolerate the side effects from, treatment with serotonin-norepinephrine reuptake inhibitors (SNRIs), such as venlafaxine, sibutramine, duloxetine, atomoxetine, desvenlafaxine, milnacipran, and levomilnacipran;
1.59 Method 1 or any of Methods 1.1-1.58, wherein said patient is not responsive to or cannot tolerate the side effects from, treatment with antipsychotic agents, such as clomipramine, risperidone, quetiapine and olanzapine;
1.60 Method 1 or any of Methods 1.1-1.59, wherein said patient was previously treated with another pain-relieving medication, and the patient did not respond adequately to said medication, e.g., the patient's pain did not abate sufficiently, or the patient suffered from side-effects which precluded continued treatment;
1.61 Method 1.60, wherein the patient developed or became at risk of developing an addiction to said other pain-relieving medication;
1.62 Method 1.60 or 1.61, wherein said other pain-relieving medication is selected from non-opiate analgesics (e.g., non-steroidal anti-inflammatory medications, such as ibuprofen, naproxen, ketoprofen, flurbiprofen, fenoprofen, oxaprozin, meclofenamate, mefenamic acid, phenylbutazone, indomethacin, ketorolac, diclofenac, sulindac, etodolac, tolmetin, nabumetone, piroxicam, acetaminophen, aspirin, celecoxib, rofecoxib, valdecoxib, parecoxib, lumiracoxib, etoricoxib, firocoxib), opiate analgesics (e.g., morphine, codeine, oxycodone, hydrocodone, hydromorphone, oxymorphone, buprenorphine, fentanyl, levorphanol, meperidine, nalbuphine, pentazocine, tramadol, methadone), and topical anesthetics (e.g., benzocaine, lidocaine, procaine, bupivacaine, tetracaine) or other medications (e.g., tricyclic antidepressants or anticonvulsants, such as amitriptyline, desipramine, duloxetine, pregabalin, gabapentin, valproate, carbamazepine, phenytoin);
1.63 Any of the foregoing methods, wherein the effective amount is 1 mg-1000 mg, preferably 2.5 mg-50 mg, or for a long-acting formulation, 25 mg-1500 mg, for example, 50 mg to 500 mg, or 250 mg to 1000 mg, or 250 mg to 750 mg, or 75 mg to 300 mg, measured as the equivalent of the free base of the compound;
1.64 Any of the foregoing methods, wherein the effective amount is 1 mg-100 mg per day, preferably 2.5 mg-50 mg per day, measured as the equivalent of the free base of the compound.
1.65 Any of the foregoing methods, wherein the effective amount of the Compound of the Invention is 1 mg-1000 mg, for example 2.5 mg-50 mg, or for a long-acting formulation, 25 mg-1500 mg, for example, 50 mg to 500 mg, or 250 mg to 1000 mg, or 250 mg to 750 mg, or 75 mg to 300 mg, measured as the equivalent of the free base of the compound;
1.66 Any of the foregoing methods, wherein the effective amount of the Compound of the Invention is 1 mg-100 mg per day, for example 2.5 mg-50 mg per day, measured as the equivalent of the free base of the compound;
1.67 Any of the foregoing methods, wherein the effective amount of the Compound of the Invention is 1 mg-5 mg, preferably 2.5-5 mg, per day, measured as the equivalent of the free base of the compound;
1.68 Any of the foregoing methods, wherein the effective amount of the Compound of the Invention is 2.5 mg or 5 mg, per day, measured as the equivalent of the free base of the compound;
1.69 Method 1 or any of 1.1-1.68, wherein the compound of Formula I is administered in the form of a pharmaceutical composition comprising the compound of Formula I in admixture with a pharmaceutically acceptable diluent or carrier;
1.70 Method 1.69, wherein the compound of Formula I is in pharmaceutically acceptable salt form in admixture with a pharmaceutically acceptable diluent or carrier;
1.71 Method 1.69 or 1.70, wherein the pharmaceutical composition is a sustained release or delayed release formulation, e.g., according to Pharmaceutical Composition 1-A as described herein;
1.72 Method 1.69, 1.70, or 1.71, wherein the pharmaceutical composition comprises the Compound of Formula I in a polymeric matrix, e.g., according to Pharmaceutical Composition 1-B as described herein;
1.73 Any of Methods 1.69-1.72, wherein the pharmaceutical composition is formulated as an osmotic controlled release oral delivery system, e.g., according to Pharmaceutical Composition 1-C or any of P.1 to P.7, as described herein;
1.74 Any of Methods 1.69-1.72, wherein the pharmaceutical composition is in the form of a tablet or capsule;
1.75 Any of Methods 1.69-1.72, wherein the pharmaceutical composition is formulated for oral, sublingual, or buccal administration;
1.76 Any of Methods 1.69-1.72, wherein the pharmaceutical composition is a rapidly-dissolving oral tablet (e.g., a rapidly dissolving sublingual tablet);
1.77 Any of Methods 1.69-1.72, wherein the pharmaceutical composition is formulated for intranasal or intrapulmonary administration (e.g., as an aerosol, mist, or powder for inhalation);
1.78 Any of Methods 1.69-1.72, wherein the pharmaceutical composition is formulated for administration by injection, for example, as a sterile aqueous solution;
1.79 Method 1.78, wherein the pharmaceutical composition is formulated for intravenous, intrathecal, intramuscular, subcutaneous or intraperitoneal injection
1.80 Any of Method 1 or 1.1-1.79, wherein the method further comprises the concurrent administration of one or more other therapeutic agents, e.g., administered simultaneously, separately or sequentially;
1.81 Method 1.80, wherein the one or more additional therapeutic agents comprise an opiate or opioid agent, e.g., an opioid agonist or partial opioid agonist, for example, a mu-agonist or partial agonist, or a kappa-agonist or partial agonist, including mixed agonist/antagonists (e.g., an agent with partial mu-agonist activity and kappa-antagonist activity);
1.82 Method 1.80, wherein the additional opiate or opioid agent is selected from the group consisting of morphine, codeine, thebaine, oripavine, morphine dipropionate, morphine dinicotinate, dihydrocodeine, buprenorphine, etorphine, hydrocodone, hydromorphone, oxycodone, oxymorphone, fentanyl, alpha-methylfentanyl, alfentanyl, trefantinil, brifentanil, remifentanil, ocfentanil, sufentanil, carfentanyl, meperidine, prodine, promedol, propoxyphene, dextropropoxyphene, methadone, diphenoxylate, dezocine, pentazocine, phenazocine, butorphanol, nalbuphine, levorphanol, levomethorphan, tramadol, tapentadol, and anileridine, or any combinations thereof;
1.83 Method 1.80, wherein the additional opiate or opioid agent is buprenorphine, optionally, wherein said method does not include co-treatment with an anxiolytic agent, e.g., a GABA compound or benzodiazepine;
1.84 Method 1.80, wherein the additional opiate or opioid agent is an opioid receptor antagonist or inverse agonist, e.g., a full opioid antagonist, for example, selected from naloxone, naltrexone, nalmefene, methadone, nalorphine, levallorphan, samidorphan, nalodeine, cyprodime, or norbinaltorphimine;
1.85 Any of Methods 1.80-1.84, wherein an additional therapeutic agent is a NMDA receptor antagonist, e.g., administered simultaneously, separately or sequentially;
1.86 Method 1.85, wherein the NMDA receptor antagonist is selected from the group consisting of ketamine (e.g., S-ketamine and/or R-ketamine), hydroxynorketamine, memantine, dextromethorphan, dextroallorphan, dextrorphan, amantadine, and agmatine, or any combination thereof;
1.87 Any of Methods 1.80-1.86, wherein an additional therapeutic agent is selected from compounds that modulate GABA receptor activity, GABA-B receptor agonists, 5-HT receptor modulators, 5-HT_{1A} receptor agonists, 5-HT_{2A} receptor antagonists, 5-HT_{2A} receptor inverse agonists, melatonin receptor agonists, ion channel modulators, ion channel blockers, SARIs (serotonin-2 receptor antagonists/reuptake inhibitors), orexin receptor antagonists, H3 receptor agonists, H3 receptor antagonists, noradrenergic receptor agonists, noradrenergic receptor antagonists, galanin receptor agonists, CRH receptor antagonists, human growth hormone, growth hormone receptor agonists, estrogen, estrogen receptor agonists, neurokinin-1 drugs, anti-depressants, opioid receptor agonists, partial opioid receptor agonists, opioid receptor antagonists, opioid receptor inverse agonists, nociception/orphanin receptor agonists, typical antipsychotic agents, atypical antipsychotic agents, serotonin HT6 receptor antagonists, and an mGluR-2, -3 or -5 receptor agonists or antagonists;
1.88 Any of Methods 1.80-1.87, wherein an additional therapeutic agent is a compound that modulates GABA activity (e.g., enhances the activity and facilitates GABA transmission);
1.89 Method 1.88, wherein the additional therapeutic agent is a GABA compound selected from a group consisting of doxepin, alprazolam, bromazepam, clobazam, clonazepam, clorazepate, diazepam, flunitrazepam, flurazepam, lorazepam, midazolam, nitrazepam, oxazepam, temazepam, triazolam, indiplon, zopiclone, eszopiclone, zaleplon, Zolpidem, gaboxadol, vigabatrin, tiagabine, EVT 201 (Evotec Pharmaceuticals), and estazolam;
1.90 Any of Methods 1.80-1.89, wherein an additional therapeutic agent is a 5-HT_{2A} receptor antagonist, optionally selected from pimavanserin, ketanserin, risperidone, eplivanserin, volinanserin, pruvanserin, MDL 100907 (Sanofi-Aventis, France), HY 10275 (Eli Lilly), APD 125 (Arena Pharmaceuticals, San Diego, CA), and AVE8488 (Sanofi-Aventis, France);
1.91 Any of Methods 1.80-1.90, wherein an additional therapeutic agent is a melatonin receptor agonist, optionally selected from a group consisting of one or more of melatonin, ramelteon (ROZEREM®, Takeda Pharmaceuticals, Japan), VEC-162 (Vanda Pharmaceuticals, Rockville, MD), PD-6735 (Phase II Discovery), and agomelatine;
1.92 Any of Methods 1.80-1.91, wherein an additional therapeutic agent is an ion channel blocker, optionally selected from lamotrigine, gabapentin and pregabalin;
1.93 Any of Methods 1.80-1.92, wherein an additional therapeutic agent is an orexin receptor antagonist, optionally selected from a group consisting of orexin, SB-334867-a (GlaxoSmithKline, UK), and GW649868 (GlaxoSmithKline);
1.94 Any of Methods 1.80-1.93, wherein an additional therapeutic agent is a serotonin-2 receptor antagonist/reuptake inhibitor (SARI), optionally selected from a group consisting of one or more Org 50081 (Organon-Netherlands), ritanserin, nefazodone, serzone, and trazodone;
1.95 Any of Methods 1.80-1.94, wherein an additional therapeutic agent is a 5-HT_{1A} agonist, optionally selected from a group consisting of one or more of repinotan, sarizotan, eptapirone, buspirone and MN-305 (MediciNova, San Diego, CA);
1.96 Any of Methods 1.80-1.95, wherein an additional therapeutic agent is a neurokinin-1 drug, optionally wherein the drug is Casopitant;
1.97 Any of Methods 1.80-1.96, wherein an additional therapeutic agent is an antipsychotic agent, optionally wherein the antipsychotic agent is selected from a group consisting of chlorpromazine, haloperidol, droperidol, fluphenazine, loxapine, mesoridazine, molindone, perphenazine, pimozide, prochlorperazine promazine, thioridazine, thiothixene, trifluoperazine, brexpiprazole, cariprazine, asenapine, lurasidone, clozapine, aripiprazole, olanzapine, quetiapine, risperidone, ziprasidone and paliperidone;
1.98 Any of Methods 1.80-1.97, wherein an additional therapeutic agent is an anti-depressant agent, optionally wherein the anti-depressant agent is selected from amitriptyline, amoxapine, bupropion, citalopram, clomipramine, desipramine, doxepin, duloxetine, escitalopram, fluoxetine, fluvoxamine, imipramine, isocarboxazid, maprotiline, mirtazapine, nefazodone, nortriptyline, paroxetine, phenelzine sulfate, protriptyline, sertraline, tranylcypromine, trazodone, trimipramine, and venlafaxine;
1.99 Any of Methods 1.80-1.98, wherein an additional therapeutic agent is an atypical antipsychotic agent, optionally wherein the agent is selected from a group consisting of brexpiprazole, cariprazine, asenapine, lurasidone, clozapine, aripiprazole, olanzapine, quetiapine, risperidone, ziprasidone, and paliperidone;
1.100 Any of Methods 1.80-1.99, wherein an additional therapeutic agent is an atypical stimulant, optionally selected from modafinil, adrafinil, and armodafinil;
1.101 Any of Methods 1.80-1.100, wherein an additional therapeutic agent is an anti-Parkinson's agent, optionally selected from L-dopa, co-careldopa, duodopa, stalevo, Symmetrel, benztropine, biperiden, bromocriptine, entacapone, pergolide, pramipexole, procyclidine, ropinirole, selegiline and tolcapone.
1.102 Any of Methods 1.80-1.101, wherein any one or more additional therapeutic agents are administered to the patient as a single Pharmaceutical Composition, such as a depot composition, as hereinbefore described;
1.103 Any of Methods 1.80-1.102, wherein any one or more additional therapeutic agents are administered to the patient as a separate Pharmaceutical Composition, such as wherein one is a depot composition, as hereinbefore described, and the other is not (e.g., an oral dosage form).

As noted above, the Compounds of the Invention may be particularly useful because of their potential as biased mu-opioid receptor ligands. Depending on the cell type, or even within the same cell type, the intracellular domain of an activated mu opioid receptor can interact either with inhibitory G proteins or with beta-arrestin. The binding of a non-biased agonist to the mu-opioid receptor will result in approximately equal activation of both G-protein signaling and beta-arrestin signaling.

In contrast, when a biased agonist binds to a mu opioid receptor, it binds in such a way as to bias the intracellular domain of the receptor to interact with the G protein instead of the beta-arrestin. Thus, the Compounds of the Invention may act as partial or full agonists of the mu-opioid receptor's G-protein coupled signaling, but as an antagonist of the receptor's beta-arrestin signaling. This is in contrast to traditional opioid agonists, such as morphine and fentanyl, which tend to strongly activate both G-protein signaling and beta-arrestin signaling pathways. The activation of beta-arrestin signaling by such drugs is thought to mediate the gastrointestinal dysfunction, addiction, and respiratory depression effects typically mediated by opioid drugs, while the analgesic and anesthetic effects of mu-opioid receptor agonists are mediated by the G-protein signaling pathway.

Furthermore, because biased agonists antagonize the beta-arrestin pathway, they are known to be generally useful in treating opioid overdose—by reversing the respiratory depression caused by the opioid. Beneficially, however, they will do so while still providing pain relief. Biased beta-arrestin antagonists are expected to be useful in treating opioid overdose, because they will inhibit the most severe opioid adverse effects but still provide pain relief.

The United States is currently in the throes of a widespread opioid abuse epidemic that began in the late 1990's and is fueled by a combination of overprescribed prescription opioids (such as oxycodone, sold as OxyContin by Purdue Pharma), cheap imported illicit heroin, and a combination of licit and illicit fentanyl. While heroin and oxycodone (along with codeine, hydrocodone, hydromorphone, oxymorphone, and several other drugs) are natural or semi-synthetic analogs of morphine, fentanyl was the first and most prominent of a newer class of synthetic opioids. Unlike the natural and semi-synthetic opioids, fentanyl and fentanyl analogs do not have the complete classic pentacyclic core skeleton of morphine. Instead, fentanyl and fentanyl analogs share a common 4-aminophenyl(piperidine) core. The most common fentanyl analogs are sufentanil, alfentanil, remifentanil, and carfentanil.

Fentanyl and its analogs are substantially more potent than both morphine and heroin, due to either stronger mu-opioid receptor binding, higher lipophilicity, or both. The higher lipophilicity of these drugs, compared to morphine and heroin, results in them crossing the blood-brain barrier much faster, so that even with comparable receptor binding they are more potent. Fentanyl is generally considered about 50 times more potent than heroin and 100 times more potent than morphine (some sources indicate it as 150 times more potent than morphine). Sufentanil is considered 5 to 10 times more potent than fentanyl, and carfentanil about 100 times more potent than fentanyl (and thus 10,000 times more potent than morphine).

Because of its extremely high potency, and widespread cheap availability, it has become increasingly common for amphetamines, heroin and other street drugs to be adulterated with varying, and unpredictable, amounts of fentanyl. As a result of this and other trends, fentanyl has become a leading cause of opioid overdose in the United States, and especially, of opioid-related deaths. By 2016, fentanyl was the cause of at least 50% of opioid deaths, rising to more than 70% of deaths in 2017 and 2018. See Torralva & Janowsky, J. Pharmacol. Exp. Ther. 371:453-475 (2019). In fact, the rate of amphetamine overdoses has increased substantially in the last few years, driven primarily by the adulteration of amphetamine with fentanyl. In only a 5-year period, there was a 4-fold rise in amphetamine mortality, primarily linked to fentanyl adulteration.

Only three fentanyl analogs are approved for human use (sufentanil, alfentanil, remifentanil) while one is approved for veterinary use only (carfentanil). Nevertheless, these and a host of other novel synthetic fentanyl analogs have been found as adulterants in numerous street drugs, including amphetamines, heroin, cocaine, alprazolam (Xanax) and hydrocodone/paracetamol (Norco). See Armenian et al., Neuropharmacology (2017). Until 2013, there were only sporadic outbreaks of fentanyl or fentanyl analogs contaminating the U.S heroin supply, but since then, such compounds have widely infiltrated North America, contaminating both heroin and cocaine. Deaths from fentanyl-laced heroin and cocaine doubled from 2012 to 2014. Street-purchased counterfeit Xanax and Norco caused two outbreaks in California in 2015-2016. The adulteration of non-opioid drugs with fentanyl and fentanyl analogs is particularly concerning because the users of such drugs are likely to be opioid naïve (thus having little or no established drug tolerance), and thus have significantly worse clinical outcomes. As testing for standard fentanyl analogs became more widespread (both in the medical setting and the forensic setting), illicit manufacturers began switching to novel synthetic fentanyl derivatives to evade detection, and today, numerous such illicit compounds are known and available on the black market from manufacturers in China and elsewhere. At least 21 synthetic opioid compounds are scheduled by the U.S. Drug Enforcement Administration today.

Because of its high potency and high lipophilicity, fentanyl-induced overdose is much more difficult to treat than morphine, heroin or oxycodone overdose. Fentanyl has an extraordinarily rapid onset of action, which makes reversal via mu-receptor antagonist (e.g., naloxone or naltrexone) treatment difficult in the outpatient setting (response time for EMS or police often being longer than the time for severe respiratory depression to develop). Larger doses of mu-receptor antagonists are also required to reverse fentanyl overdose, and there are limits on the rate and dose of mu-opioid antagonists that can be safely administered. While morphine takes an average of 19 minutes to reach 80% of peak effect, fentanyl produces severe respiratory depression much more rapidly.

Even more worrying, however, is that fentanyl and its analogs have an additional mechanism of action that has become extremely important in the ongoing opioid epidemic. While all opioids cause respiratory depression via mu-opioid receptor activation of the beta-arrestin signaling pathway in the brain, for reasons that are not yet entirely clear, fentanyl and its analogs can also cause a rapid combination of vocal cord closure (laryngospasm) and severe muscle rigidity in the chest wall and diaphragm. This can result from intravenous, transdermal, or inhalational administration of fentanyl and fentanyl analogs. Neither morphine, heroin, nor any other opioids having the classic morphine skeleton have this property. This severe chest wall rigidity has been referred to as fentanyl-induced respiratory muscle rigidity (FIRMR) (or simply fentanyl-induced muscle rigidity FIMR), and the combination of FIRMR and laryngospasm is clinically known as wooden chest syndrome (WCS). WCS can develop within only 1-2 minutes of injection of fentanyl, fentanyl analogs, or heroin or other drugs laced with fentanyl or its analogs. WCS has been demonstrated following as little as 50 micrograms of intravenous fentanyl.

The primary cause of mortality in WCS appears to be from the mechanical disruption of ventilation caused by closure of the glottic structures and upper airway. Laryngospasm is defined as the involuntary closure or occlusion of the glottic opening, and this is controlled by the intrinsic muscles of the larynx. These muscles are innervated by both sympathetic (adrenergic) and parasympathetic (cholinergic) nerve fibers, and the ultimate activity of these muscles is thus determined by the balance of sympathetic and parasympathetic input.

While FIRMR and WCS have long been known in the surgical anesthetic community (because it commonly occurs within the therapeutic dose range for surgical anesthesia), these conditions are not well-known in the first responder or emergency medical communities. This often leads to rapid death of drug abusers because those treating them are not aware of these effects of fentanyl (often compounded also by the lack of the patient's knowledge of having taken something having fentanyl in it). Numerous eyewitness and survivor accounts of overdoses report a very rapid onset of cyanosis, loss of consciousness, severe muscle rigidity, and seizure like behavior, immediately following injection of drug. The rapid onset of death is very unlike the respiratory depression normally associated with morphine, heroin, and oxycodone overdoses. Indeed, mechanical failure of respiration in a fentanyl or fentanyl analog overdose usually develops less than 2 minutes after drug administration and presents prior to centrally-mediated respiratory depression (50% drop in respiratory mechanics takes 7-9 minutes to develop).

Even more worrying, the standard first-line therapies for opioid overdose—naloxone, naltrexone, and nalmefene—are not effective in reversing these fentanyl-induced effects. The severe chest wall rigidity also compromises the effectiveness of chest compressions in cardiopulmonary resuscitation. As a result, while the ratio of emergency room visits to death for heroin-related overdose has been reported as about 10:1, the ratio is only 1:1 for fentanyl-related overdoses.

The standard dose of intravenous naloxone administered for opioid overdose is 0.4 to 2 mg, with additional doses at 2-to-3-minute intervals, up to a maximum of 10 mg. However, intranasal naloxone, which is widely used by first responders, is recommended for only a 4 mg maximum total dose. See, e.g., Williams et al., Prehospital Emergency Care 23(6):749-63 (2019). In one study, however, it was found that while the upper airway effects of morphine could be fully blocked by a dose of 0.1 mg/kg of naloxone (e.g., 7 mg for a 70-kg person), to fully block the upper airway effects of fentanyl required from 0.8 to 1.6 mg/kg of naloxone (56 to 112 mg for a 70-kg person). A study examining a 2006 fentanyl overdose outbreak reported that 0.4 to 12 mg of naloxone was administered to patients in a hospital emergency room, with only 15% patients responding to a 0.4 mg dose, and 6 patients out of 26 requiring at least 6 mg to reverse respiratory depression. In another study examining 18 patients who overdosed on counterfeit hydrocodone/paracetamol contaminated with fentanyl, 0.4 to 8 mg intravenous bolus injections of naloxone were required, and 4 of the patients required naloxone infusions lasting 26-40 hours.

Unfortunately, however, high doses of naloxone are not practical for therapeutic use because the rapid injection of as little as 0.4 mg of naloxone (0.0057 mg/kg for a 70 kg adult) in active opioid users commonly results in laryngospasm, pulmonary edema, hemodynamic instability, and cardiac arrythmia (all due to catecholamine release). High-dose naloxone treatment is therefore contraindicated, especially in the field. Thus, in the field-without additional medical and pharmacological support—it is normally quite difficult, if not impossible to, to use naloxone to reverse fentanyl-induced overdose before it becomes fatal.

It is clear that WCS is not simply the result of mu-opioid receptor agonism-since other powerful mu-opioid agonists do not cause WCS (e.g., morphine), and since powerful mu-opioid antagonists (e.g., naloxone) do not reverse WCS at normal dose ranges. Thus, fentanyl and its analogs must cause WCS by some other mechanism which involves other neurotransmitter systems.

There is evidence, both from in vitro studies and from various animal models, which indicates that fentanyl exerts these effects via the stimulation of noradrenergic activity, and possibly cholinergic activity, in the locus coeruleus (LC) region of the brain. Without being bound by theory, it is believed that in the LC, fentanyl acts as an agonist of mu-opioid receptors, and the resulting hyperpolarization of the LC neuron results in efferent noradrenergic neuron activity, specifically, in coerulospinal fibers connected to spinal motor neurons terminating in the chest wall and abdomen, as well as laryngeal nerve fibers contributing to the vagal nerve via the superior cervical and middle cervical ganglia. These laryngeal nerve fibers directly innervate the intrinsic muscles of the larynx.

The role of the alpha1-adrenergic receptor, in particular, has been indicated by animal experiments demonstrating that the selective alpha1-adrenergic antagonist prazosin, administered intravenously ten minutes prior to fentanyl, inhibits the development of FIMR, and the same result occurs with ablation of the LC region of the brain. Other studies show that intrathecal administration of prazosin at the L3 spinal level also inhibits FIMR, but the administration of yohimbine, an alpha2-adrenergic antagonist, does not. There has also been some animal evidence that fentanyl itself is an antagonist of the alpha1-adrenergic receptor, although weakly, and with selectivity for the alpha1B and alpha1A receptors (rather than the alpha1D receptor). Unfortunately, these studies do not directly predict the beneficial use of alpha1-adrenergic antagonists in treating opioid overdose, because the corresponding human doses used in the animal studies would result in lethal hypotension in humans.

There is also increasing evidence for an intermediate role for GABA interneurons in the pathogenesis of WCS. GABA interneurons are part of an inhibitory network throughout the brain, and they are particularly abundant in the LC. The LC is responsible for maintaining basal skeletal muscle tone in the torso via the noradrenergic activation of spinal motor neurons, but norepinephrine release from the LC presynaptic terminals is inhibited by the GABA efferent signaling. Inhibition of the GABA interneurons, therefore, results in increased skeletal muscle tone via increased LC noradrenergic activity. Without being bound by theory, it is believed that fentanyl binds to mu-opioid receptors on GABA interneurons, and that this results in inhibition of GABA interneuron afferents, resulting in release of the inhibition on LC sympathetic neurons.

There is also evidence that LC neurons are also high in muscarinic and nicotinic acetylcholine receptors. It is believed that as the LC receives cholinergic input from other brain regions, such as the pontine reticular formation, fentanyl-induced mu-receptor agonism in these neighboring regions may stimulate acetylcholine release, which results in further stimulation of norepinephrine release by the LC. There is also some evidence that fentanyl acts directly as an M3 muscarinic receptor antagonist, which may result in inhibition of parasympathetic tone at the laryngeal intrinsic muscles, further increasing the spasm resulting from sympathetic activation of these muscles.

NMDA and non-NMDA glutamate receptor activity has also been implicated in the pathogenesis of WCS.

Because of the intermediate role of these other neurotransmitters (e.g., norepinephrine, acetylcholine, GABA, etc.) in the pathogenesis of WCS, a further reason for the failure of response of WCS to mu-opioid antagonist treatment might be that once these indirect fentanyl-stimulated effects are initiated (by mu-receptor agonism), mere mu-receptor antagonism cannot reverse the effects already set in motion.

Finally, there is also evidence that fentanyl, but not morphine, has some activity as a norepinephrine reuptake inhibitor. It has been shown in various neural cell lines that this effect is not antagonized by naloxone, indicating that it is not an indirect effect of mu-receptor agonism. Thus, it is also possible that fentanyl is exerting a direct effect on neurons in the LC and stimulating hyperactivity of the muscles involved in FIMR and WCS.

Thus, there remains a need for therapeutic agents which are particularly suited to reversing the effects of an acute fentanyl overdose or fentanyl analog overdose. Fentanyl analogs include, but are not limited to, the compounds sufentanil, alfentanil, remifentanil, carfentanil, as well as derivatives of these compounds, as further explained herein. Fentanyl and fentanyl analogs are collectively referred to herein as “F/FA.”

Without being bound by theory, it is believed that the Compounds of the Invention may, due to their potent 5-HT_{2A}, D₁ and Mu opioid modulation activity, and especially due to their biased mu-opioid receptor activity, be unexpectedly effective in reversing the symptoms of F/FA overdose, especially respiratory depression, chest wall rigidity and laryngospasm. This is particularly believed to be due to these compounds' activity as mu-receptor antagonists via the beta-arrestin signaling. It is also believed that these compounds' activity as alpha1-adrenergic antagonists, as indirect NMDA and AMPA antagonists, and potentially due to indirect effects on GABA expressing neurons. These properties are highly unique and are not shared by the traditional mu-opioid receptor antagonists which are used for both opioid overdose treatment and surgical reversal of opioid agonism, such as naloxone.

The compounds disclosed herein are also highly beneficial in treating acute overdose and chronic opioid addiction because they do not induce opioid withdrawal symptoms in the manner that opioid cessation or opioid antagonist treatment may. Opioid withdrawal syndrome can be very severe on addicted patients, and can include symptoms such as tachycardia, nausea, vomiting, diarrhea, extreme anxiety, restless legs, muscle aches, and profuse sweating. These withdrawal symptoms are the result of the body's adaptation to the presence of opioids resulting in tolerance and physical dependence. In severe cases, sudden cessation of opioid abuse or treatment with opioid antagonists can result in withdrawal symptoms lasting for weeks or months. Administration of opioid antagonists, such as naloxone or naltrexone, especially in high doses, can precipitate acute withdrawal effects, especially in patients suffering from an acute overdose with F/FA. In patients suffering from overdose with weaker opioid agonists, such as heroin, antagonist treatment can be administered using small repeated doses in order to avoid or minimize such withdrawal syndromes. However, in an acute F/FA overdose, such small doses of antagonist are ineffective, and thus, in order to have any chance of reversing the overdose, it is often impossible to avoid severe withdrawal with traditional antagonist treatments.

In a further embodiment of the third aspect, the present disclosure provides further embodiments of Method 1, wherein the method is a method for one or more of the following (Method 1-A):
(a) treating or reversing F/FA overdose;
(b) treating or reversing F/FA-induced respiratory depression;
(c) treating or reversing F/FA-induced muscle rigidity;
(d) treating or reversing F/A-induced laryngospasm;
(e) reversing or inhibiting binding of F/FA to mu-opioid receptors in the central nervous system (e.g., in the locus coeruleus);
(f) inhibiting F/FA-induced beta-arrestin signaling in the central nervous system (e.g., in the locus coeruleus);
(g) preventing death from F/FA overdose; and
(h) anesthetic recovery (e.g., following surgery);
   the method comprising administering to a patient in need thereof an effective amount of a Compound of the Invention, or a pharmaceutical composition comprising a Compound of the Invention.

Further exemplary embodiments of this aspect of Method 1, include the following: 1.104 Method 1 or any of 1.1-1.103, wherein the method is a Method 1-A;
1.105 Method 1-A, or 1.104, wherein the patient is unconscious;
1.106 Method 1-A, or 1.104 or 1.105, wherein the patient is suspected of suffering from an acute F/FA overdose;
1.107 Method 1-A or any of Methods 1.104-1.106, wherein the patient demonstrates chest wall rigidity;
1.108 Method 1-A or any of Methods 1.104-1.107, wherein the patient demonstrates laryngospasm;
1.109 Method 1-A or any of Methods 1.104-1.08, wherein the patient is diagnosed with or suspected or having wooden chest syndrome (WCS);
1.110 Method 1-A or any of Methods 1.104-1.08, wherein the patient is diagnosed with or suspected or having fentanyl-induced muscle rigidity (FIMR) or fentanyl-induced respiratory muscle rigidity (FIRMR) (wherein said FIMR or FIRMR is caused by fentanyl or by a fentanyl analog);
1.111 Method 1-A or any of Methods 1.104-1.110, wherein the patient is in a non-hospital or non-emergency clinic setting;
1.112 Method 1-A or any of Methods 1.104-1.111, wherein the patient is suspected of suffering from opioid use disorder, or has a history of opioid use disorder;
1.113 Method 1-A or any of Methods 1.104-1.112, wherein the patient is suspected of being a naïve opioid user;
1.114 Method 1-A or any of Methods 1.104-1.113, wherein the patient has, or is suspected of having, overdosed on a licit or illicit drug contaminated with or admixed with F/FA (e.g., morphine, heroin, codeine, hydrocodone, oxycodone, hydromorphone, marijuana or cannabis products, tetrahydrocannabinol, cocaine, amphetamine, methamphetamine, methylenedioxymethamphetamine, alprazolam, or other illicit or licit drugs);
1.115 Method 1-A, or any of Methods 1.104-1.114, wherein the F/FA is or was administered as general anesthesia (e.g., surgical anesthesia);
1.116 Method 1.115, wherein the general anesthesia further comprises or comprised one or more of an inhalational anesthetic (e.g., isoflurane, sevoflurane, desflurane, nitrous oxide, halothane, methoxyflurane), another opioid agonist (e.g., morphine, oxycodone), a sedative or hypnotic (e.g., propofol, midazolam, ketamine, etomidate), or a muscle relaxant (e.g., atracurium, mivacurium, pancuronium, rocuronium, vecuronium, cisatracurium, succinylcholine);
1.117 Method 1.115 or 1.116, wherein the patient has difficulty emerging from anesthesia, e.g., due to persistent respiratory depression;
1.118 Method 1-A or any of Methods 1.104-1.117, wherein the patient has not responded to, or has not responded adequately to (e.g., with respect to signs or symptoms of respiratory depression) a single dose of a mu-opioid antagonist (e.g., naloxone or naltrexone, e.g., 0.1 to 4 mg) administered by any route (e.g., intranasal, intravenous, subcutaneous, or intramuscular);
1.119 Method 1-A or any of Methods 1.104-1.117, wherein the patient has not responded to, or has not responded adequately to (e.g., with respect to signs or symptoms of respiratory depression) multiple doses of a mu-opioid antagonist (e.g., naloxone or naltrexone, e.g., 0.4 to 20 mg in total) administered by any route (e.g., intranasal, intravenous, subcutaneous, or intramuscular);
1.120 Method 1-A or any of Methods 1.104-1.119, wherein the patient has experienced recrudescence of respiratory depression following a single dose or multiple doses of a mu-opioid antagonist administered by any route (e.g., naloxone or naltrexone, e.g., intranasal, intravenous, subcutaneous, or intramuscular);
1.121 Method 1-A or any of Methods 1.104-1.120, wherein the patient has experienced one or more opioid withdrawal symptoms or other adverse events (e.g., agitation, combativeness, laryngospasm, pulmonary edema, hemodynamic instability, or cardiac arrythmia) following a single dose or multiple doses of a mu-opioid antagonist administered by any route (e.g., naloxone or naltrexone, e.g., intranasal, intravenous, subcutaneous, or intramuscular);
1.122 Any of Methods 1.118-1.121, wherein said mu-opioid antagonist is naloxone, naltrexone, or nalmefene;
1.123 Any of Methods 1.118-1.121, wherein said mu-opioid antagonist is naloxone;
1.124 Method 1-A or any of 1.104-1.123, wherein the patient has been administered at least one dose of naloxone and has suffered from one or more opioid withdrawal symptoms or adverse events such that further doses of naloxone are contraindicated;
1.125 Method 1-A or any of 1.104-1.124, wherein the use of naloxone is contraindicated for any reason;
1.126 Method 1-A or any of 1.104-1.125, wherein said patient has previously suffered from an opioid overdose;
1.127 Method 1-A or any of 1.104-1.126, wherein said patient is confirmed to be suffering from F/FA overdose by toxicological or forensic methods (e.g., by confirming the presence of F/FA in the patient's blood, or in the patient's drugs or drug paraphernalia);
1.128 Method 1-A or any of 1.104-1.127, wherein the effective amount of the Compound of Formula I is an amount effective to reverse one or more of: respiratory arrest, respiratory depression, skeletal muscle spasm, chest wall rigidity, laryngospasm, pupillary constriction, cardiac arrest, bradycardia, or unconsciousness;
1.129 Method 1-A or any of 1.104-1.128, wherein the effective amount of the Compound of the Invention is 0.1 mg-200 mg, for example, 1-200 mg, or 10-150 mg, or 25-100 mg, or 50-100 mg, or 75-100 mg, or 25-75 mg, or 25-50 mg, or 1-50 mg, or 1-25 mg, 0.1 to 50 mg, 2.5 mg-50 mg, or for a long-acting formulation, 25 mg-1500 mg, for example, 50 mg to 500 mg, or 250 mg to 1000 mg, or 250 mg to 750 mg, or 75 mg to 300 mg;
1.130 Method 1.129, wherein the effective amount is administered as a single dose;
1.131 Method 1.129, wherein the effective amount is administered in two or more doses over a period of less than 30 minutes (e.g., less than 20 minutes, or less than 15 minutes, or less than 10 minutes);
1.132 Method 1-A or any of 1.104-1.131, wherein the effective amount of the Compound of the Invention is administered by intranasal administration (e.g., as an aerosol, mist, or powder for inhalation);
1.133 Method 1-A or any of 1.104-1.131, wherein the effective amount of the Compound of the Invention is administered across the oral mucosa, such as, by a rapidly-dissolving oral tablet (e.g., a rapidly dissolving sublingual tablet);
1.134 Method 1-A or any of 1.104-1.131, wherein the effective amount of the Compound of the Invention is administered by injection (e.g., intravenous, intramuscular, intrathecal, intraperitoneal, or subcutaneous injection);
1.135 Method 1-A or any of 1.104-1.134, wherein the method does not comprise the concurrent administration of any other opioid antagonist (e.g., naloxone, naltrexone, nalmefene, methadone, nalorphine, levallorphan, samidorphan, nalodeine, cyprodime, or norbinaltorphimine);
1.136 Method 1-A or any of 1.104-1.135,wherein the method comprises administering a pharmaceutical composition comprising both a Compound of Formula I, wherein R¹ is H, and a prodrug of the same Compound of Formula I (i.e., wherein R¹ is —C(O)—O—C(R^{a})(R^{b})(R^{c}), —C(O)—O—CH₂—O—C(R^{a})(R^{b})(R^{c}) or —C(R⁶)(R⁷)—O—C(O)—R⁸, as hereinbefore described);
1.137 Method 1-A or any of 1.104-1.136, wherein the Compound of the Invention is the sole pharmacological treatment for the overdose (e.g., other than supportive interventions, such as oxygen administration, cardiopulmonary resuscitation, chest compressions, and fluid administration);
1.138 Method 1-A or any of 1.104-1.137, wherein the method is a method for treating or reversing F/FA overdose;
1.139 Method 1-A or any of 1.104-1.138, wherein the method is a method for treating or reversing F/FA-induced respiratory depression;
1.140 Method 1-A or any of 1.104-1.139, wherein the method is a method for treating or reversing F/FA-induced muscle rigidity;
1.141 Method 1-A or any of 1.104-1.140, wherein the method is a method for treating or reversing F/A-induced laryngospasm;
1.142 Method 1-A or any of 1.104-1.141, wherein the method is a method for reversing or inhibiting binding of F/FA to mu-opioid receptors in the central nervous system (e.g., in the locus coeruleus);
1.143 Method 1-A or any of 1.104-1.142, wherein the method is a method for inhibiting F/FA-induced beta-arrestin signaling in the central nervous system (e.g., in the locus coeruleus);
1.144 Method 1-A or any of 1.104-1.143, wherein the method is a method for preventing death from F/FA overdose;
1.145 Method 1-A or any of 1.104-1.144, wherein the method is a method for anesthetic recovery (e.g., anesthetic emergence, such as, following surgery)
1.146 Method 1-A or any of 1.104-1.145, wherein the F/FA is any F/FA disclosed herein throughout;
1.147 Method 1-A or any of 1.104-1.146, wherein the F/FA is selected from fentanyl, sufentanil, alfentanil, remifentanil, carfentanil, thiafentanil, lofentanil, ocfentanil, trefantinil, and brifentanil;
1.148 Method 1-A or any of 1.104-1.147, wherein the F/FA is selected from fentanyl, sufentanil, alfentanil and carfentanil;
1.149 Method 1-A or any of 1.104-1.148, wherein the F/FA is fentanyl;
1.150 Method 1-A or any of 1.104-1.149, wherein the method does not cause precipitated withdrawal in the patient, e.g., withdrawal symptoms selected from tachycardia, nausea, vomiting, diarrhea, extreme anxiety, restless legs, muscle aches, and profuse sweating;
1.151 Any of Method 1-A, or 1.104-1.150, wherein the source of the F/FA is another illicit drug which is adulterated with the F/FA, such as cocaine, heroin, oxycodone, amphetamine, methamphetamine, or marijuana;
1.152 Any of Method 1-A or 1.104-1.150, wherein the method is a method of:
   (a) treating or reversing a drug overdose;
   (b) treating or reversing drug-induced respiratory depression;
   (c) treating or reversing drug-induced muscle rigidity;
   (d) treating or reversing drug-induced laryngospasm; or
   (e) preventing death from drug overdose;
      wherein the drug is an illicit drug which has been adulterated with F/FA;
1.153 Method 1.152, wherein the illicit drug is heroin, cocaine, amphetamine, methamphetamine, oxycodone, or marijuana;
1.154 Method 1.152 or 1.153, wherein the overdose, respiratory depression, muscle rigidity, laryngospasm, and/or risk of death is due primarily or exclusively to the F/FA adulterant in the illicit drug (e.g., but for the F/FA adulterant, said illicit drug would not have caused said overdose, respiratory depression, muscle rigidity, laryngospasm, and/or risk of death).

The Compounds of the present disclosure (i.e., Compounds of the Invention) and the Pharmaceutical Compositions of the present disclosure may be used in combination with a second therapeutic agent, particularly at lower dosages than when the individual agents are used as a monotherapy so as to enhance the therapeutic activities of the combined agents without causing the undesirable side effects commonly occur in conventional monotherapy. Therefore, the Compounds of the present disclosure may be simultaneously, sequentially, or contemporaneously administered with other therapeutic agents as described hereinabove, such as opiate, opioid, analgesic, anti-depressant, anti-psychotic, other hypnotic agents, and/or agents use to treat Parkinson's disease or mood disorders.

In any of the embodiments of Method 1 et seq. wherein the Compound of the present disclosure is administered along with one or more second therapeutic agents, the one or more second therapeutic agents may be administered as a part of the pharmaceutical composition comprising the Compound of the present disclosure. Alternatively, the one or more second therapeutic agents may be administered in separate pharmaceutical compositions (such as pills, tablets, capsules and injections) administered simultaneously, sequentially or separately from the administration of the Compound of the present disclosure.

In some further embodiments of the present disclosure, the Pharmaceutical Compositions of the present disclosure may be used in combination with a second therapeutic agent, particularly at lower dosages than when the individual agents are used as a monotherapy so as to enhance the therapeutic activities of the combined agents without causing the undesirable side effects, wherein the second therapeutic agent is an opioid antagonist or inverse agonist (e.g., naloxone). The Compounds of the present disclosure may be simultaneously, sequentially, or contemporaneously administered with such opioid antagonists or opioid inverse agonists.

In a fourth aspect, the present disclosure provides use of a Compound of the Invention, in the manufacture of a medicament for use according to Method 1 or any of Methods 1.1-1.154. In another embodiment, the present disclosure provides a Compound of the Invention, for use in the treatment of a disease or disorder according to Method 1 or any of Methods 1.1-1.154.

### DETAILED DESCRIPTION

It is understood that the terms “opiate” and “opioid” are distinct, in that “opiate” refers to natural products derived from the opium poppy, such as morphine, codeine and heroin, but “opioid” refers to these natural compounds as well as semi-synthetic and synthetic derivatives thereof, such as fentanyl and its analogs.

The Compound of Formula A, and related compounds, have been shown to have a variety of useful pharmaceutical properties, each of which is expected to be shared by many of the compounds of the present disclosure. Such properties, and data supporting such therapeutic efficacies, are disclosed in, for example, U.S. Pat. Nos. 10,245,260, 11,376,249, US 2021/0093634, WO 2021/154519, US 2022/0088014, WO 2020/206391, US 2022/0184072, U.S. Provisional Application No. 63/262,732, and PCT/US2022/078177, the contents of each of which are hereby incorporated by reference in their entireties.

For example, the compound of Formula A has potent 5-HT_{2A}, D₁ and Mu opioid receptor antagonism, along with moderate D₂ receptor and SERT antagonism. Furthermore, it has been unexpectedly found that such compounds may operate as “biased” Mu opioid receptor ligands. This means that when the compounds bind to Mu opioid receptors, they may operate as partial Mu agonists via G-protein coupled signaling, but as Mu antagonists via beta-arrestin signaling. This is in contrast to the traditional opioid agonists morphine and fentanyl, which tend to strongly activate both G-protein signaling and beta-arrestin signaling. The activation of beta-arrestin signaling by such drugs is thought to mediate the gastrointestinal dysfunction and respiratory suppression typically mediated by opioid drugs. The Compounds of the Invention are therefore expected to result in pain amelioration with less severe gastrointestinal and respiratory side effects than existing opioid analgesics. This effect has been shown in pre-clinical studies and Phase II and Phase III clinical trials of the biased Mu agonist oliceridine. Oliceridine has been shown to result in biased mu agonism via G-protein coupled signaling with reduced beta-arresting signaling compared to morphine, and this has been linked to its ability to produce analgesia with reduced respiratory side effects compared to morphine. Furthermore, because these compounds antagonize the beta-arrestin pathway, they are expected to be useful in treating opioid overdose, because they will inhibit the most severe opioid adverse effects while still providing pain relief. Furthermore, these compounds also have sleep maintenance effect due to their serotonergic activity. As many people suffering from chronic pain have difficulty sleeping due to the pain, these compounds can help such patients sleep through the night due to the synergistic effects of serotonergic and opioid receptor activities.

Thus, in certain embodiments, the Compounds of the present disclosure may be used in a method of treating opioid use disorder (OUD), opioid overdose, or opioid withdrawal, either alone, or in conjunction with an opioid antagonist or inverse agonist (e.g., naloxone or naltrexone). Compounds of the present disclosure are expected to show a strong ability to mitigate the dysphoria and psychiatric comorbidities associated with drug withdrawal (e.g., mood and anxiety disorders, sleep disturbances), and it also provides potent analgesia but without the adverse effects (e.g., GI effects and pulmonary depression) and abuse potential seen with other opioid treatments (e.g., oxycodone, methadone or buprenorphine). The unique pharmacologic profile of these compounds should also mitigate the risks of adverse drug-drug interactions (e.g., alcohol). These compounds are therefore particularly suited to treat opioid use disorder and the symptoms associated with opioid withdrawal. In addition, to the compounds' direct effect on mu receptor activity, the compounds' effect on serotonergic pathways results in anti-depressant, sleep maintenance, and anxiolytic effects. Because depression and anxiety are key factors leading susceptible patients to opioid use in the first place, the compounds of the present disclosure would both reduce the symptoms of opioid withdrawal at the same time that they reduce the psychiatric co-morbidities which promote opioid use—a two-pronged strategy to reduce the risk of remission. The sleep maintenance provided by these compounds would further improve the quality of life of patients undergoing OUD treatment.

In some embodiments of the present disclosure, the Compounds of the Invention have one or more biologically labile functional groups positioned within the compounds such that natural metabolic activity will remove the labile functional groups, resulting in another Compound of the Invention. For example, when group R¹ is C(O)—O—C(R^{a})(R^{b})(R^{c}), —C(O)—O—CH₂—O—C(R^{a})(R^{b})(R^{c}) or —C(R⁶)(R⁷)—O—C(O)—R⁸, under biological conditions this substituent will undergo hydrolysis to yield the same compound wherein R¹ is H, thus making the original compounds prodrugs of the compound wherein R¹ is H. Some of such prodrug compounds may have little-to-no or only moderate biological activity but upon hydrolysis to the compound wherein R¹ is H, the compound may have strong biological activity. As such, depending on the compound selected, administration of the compounds of the present disclosure to a patient in need thereof may result in immediate biological and therapeutic effect, or immediate and delayed biological and therapeutic effect, or only delayed biological and therapeutic effect. Such prodrug compounds will thus serve as a reservoir of the pharmacologically active Compound of the Invention wherein R¹ is H. In this way, some compounds of the present disclosure are particularly suited to formulation as long-acting injectable (LAI) or “Depot” pharmaceutical compositions. Without being bound by theory, an injected “depot” comprising a compound of the present disclosure will gradually release into the body tissues said compound, in which tissues said compound will be gradually metabolized to yield a Compound of the Invention wherein R¹ is H. Such depot formulations may be further adjusted by the selection of appropriate components to control the rate of dissolution and release of the compounds of the present disclosure. Such prodrug forms of compounds related to the Compound of the Invention have previously been disclosed, e.g., in US 2021/0163481, the contents of which are hereby incorporated by reference in its entirety.

If not otherwise specified or clear from context, the following terms as used herein have the following meetings.

“Alkyl” as used herein is a saturated or unsaturated hydrocarbon moiety, e.g., one to twenty-one carbon atoms in length, unless indicated otherwise; any such alkyl may be linear or branched (e.g., n-butyl or tert-butyl), preferably linear, unless otherwise specified. For example, “C₁₋₂₁ alkyl” denotes alkyl having 1 to 21 carbon atoms. In one embodiment, alkyl is optionally substituted with one or more hydroxy or C₁₋₂₂alkoxy (e.g., ethoxy) groups. In another embodiment, alkyl contains 1 to 21 carbon atoms, preferably straight chain and optionally saturated or unsaturated, for example in some embodiments wherein R₁ is an alkyl chain containing 1 to 21 carbon atoms, preferably 6-15 carbon atoms, 16-21 carbon atoms. e.g., so that together with the —C(O)— to which it attaches, e.g., when cleaved from the compound of Formula I, forms the residue of a natural or unnatural, saturated or unsaturated fatty acid.

The term “pharmaceutically acceptable diluent or carrier” is intended to mean diluents and carriers that are useful in pharmaceutical preparations, and that are free of substances that are allergenic, pyrogenic or pathogenic, and that are known to potentially cause or promote illness. Pharmaceutically acceptable diluents or carriers thus exclude bodily fluids such as example blood, urine, spinal fluid, saliva, and the like, as well as their constituent components such as blood cells and circulating proteins. Suitable pharmaceutically acceptable diluents and carriers can be found in any of several well-known treatises on pharmaceutical formulations, for example Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, New York, 1990; Katzung, ed., Basic and Clinical Pharmacology, Ninth Edition, McGraw Hill, 20037ybg; Goodman and Gilman, eds., The Pharmacological Basis of Therapeutics, Tenth Edition, McGraw Hill, 2001; Remington's Pharmaceutical Sciences, 20th Ed., Lippincott Williams & Wilkins., 2000; and Martindale, The Extra Pharmacopoeia, Thirty-Second Edition (The Pharmaceutical Press, London, 1999); all of which are incorporated by reference herein in their entirety.

The terms “purified,” “in purified form” or “in isolated and purified form” for a compound refers to the physical state of said compound after being isolated from a synthetic process (e.g., from a reaction mixture), or natural source or combination thereof. Thus, the term “purified,” “in purified form” or “in isolated and purified form” for a compound refers to the physical state of said compound after being obtained from a purification process or processes described herein or well known to the skilled artisan (e.g., chromatography, recrystallization, LC-MS and LC-MS/MS techniques and the like), in sufficient purity to be characterizable by standard analytical techniques described herein or well known to the skilled artisan.

Unless otherwise indicated, the Compounds of the Invention may exist in free base form or in salt form, such as a pharmaceutically acceptable salt form, e.g., as acid addition salts. An acid-addition salt of a compound of the invention which is sufficiently basic, for example, an acid-addition salt with, for example, an inorganic or organic acid, for example hydrochloric acid or toluenesulfonic acid. In addition, a salt of a compound of the invention which is sufficiently acidic is an alkali metal salt, for example a sodium or potassium salt, or a salt with an organic base which affords a physiologically-acceptable cation. In a particular embodiment, the salt of a Compound of the Invention is a toluenesulfonic acid addition salt.

The Compounds of the Invention are intended for use as pharmaceuticals, therefore pharmaceutically acceptable salts are preferred. Salts which are unsuitable for pharmaceutical uses may be useful, for example, for the isolation or purification of free Compounds of the Invention, and are therefore also included within the scope of the compounds of the present disclosure.

The Compounds of the Invention may comprise one or more chiral carbon atoms. The compounds thus exist in individual isomeric, e.g., enantiomeric or diastereomeric form or as mixtures of individual forms, e.g., racemic/diastereomeric mixtures. Any isomer may be present in which the asymmetric center is in the (R)-, (S)-, or (R,S)-configuration. The invention is to be understood as embracing both individual optically active isomers as well as mixtures (e.g., racemic/diastereomeric mixtures) thereof. Accordingly, the Compounds of the Invention may be a racemic mixture or it may be predominantly, e.g., in pure, or substantially pure, isomeric form, e.g., greater than 70% enantiomeric/diastereomeric excess (“ee”), preferably greater than 80% ee, more preferably greater than 90% ee, most preferably greater than 95% ee. The purification of said isomers and the separation of said isomeric mixtures may be accomplished by standard techniques known in the art (e.g., column chromatography, preparative TLC, preparative HPLC, simulated moving bed and the like).

Geometric isomers by nature of substituents about a double bond or a ring may be present in cis (Z) or trans (E) form, and both isomeric forms are encompassed within the scope of this invention.

It is also intended that the compounds of the present disclosure encompass their stable and unstable isotopes. Stable isotopes are nonradioactive isotopes which contain one additional neutron compared to the abundant nuclides of the same species (i.e., element). It is expected that the activity of compounds comprising such isotopes would be retained, and such compound would also have utility for measuring pharmacokinetics of the non-isotopic analogs. For example, the hydrogen atom at a certain position on the compounds of the disclosure may be replaced with deuterium (a stable isotope which is non-radioactive). Examples of known stable isotopes include, but not limited to, deuterium (²H or D), ¹³C, ¹⁵N, ¹⁸O. Alternatively, unstable isotopes, which are radioactive isotopes which contain additional neutrons compared to the abundant nuclides of the same species (i.e., element), e.g., ¹²³I, ¹³¹I, ¹²⁵I, ¹⁴C, ¹⁸F, may replace the corresponding abundant species of I, C and F. Another example of useful isotope of the compound of the invention is the ¹⁴C isotope. These radio isotopes are useful for radio-imaging and/or pharmacokinetic studies of the compounds of the invention. In addition, the substitution of atoms of having the natural isotopic distributing with heavier isotopes can result in desirable change in pharmacokinetic rates when these substitutions are made at metabolically liable sites. For example, the incorporation of deuterium (²H) in place of hydrogen can slow metabolic degradation when the position of the hydrogen is a site of enzymatic or metabolic activity.

Compounds of the Invention may be included as a depot formulation, e.g., by dispersing, dissolving or encapsulating the Compounds of the Invention in a polymeric matrix as described hereinbefore, such that the Compound is continually released as the polymer degrades over time. The release of the Compounds of the Invention from the polymeric matrix provides for the controlled- and/or delayed- and/or sustained-release of the Compounds, e.g., from the pharmaceutical depot composition, into a subject, for example a warm-blooded animal such as man, to which the pharmaceutical depot is administered. Thus, the pharmaceutical depot delivers the Compounds of the Invention to the subject at concentrations effective for treatment of the particular disease or medical condition over a sustained period of time, e.g., 14-180 days, preferably about 30, about 60 or about 90 days.

Polymers useful for the polymeric matrix in the Composition of the Invention (e.g., Depot composition of the Invention) may include a polyester of a hydroxyfatty acid and derivatives thereof or other agents such as polylactic acid, polyglycolic acid, polycitric acid, polymalic acid, poly-beta.-hydroxybutyric acid, epsilon.-capro-lactone ring opening polymer, lactic acid-glycolic acid copolymer, 2-hydroxybutyric acid-glycolic acid copolymer, polylactic acid-polyethylene glycol copolymer or polyglycolic acid-polyethylene glycol copolymer), a polymer of an alkyl alpha-cyanoacrylate (for example poly(butyl 2-cyanoacrylate)), a polyalkylene oxalate (for example polytrimethylene oxalate or polytetramethylene oxalate), a polyortho ester, a polycarbonate (for example polyethylene carbonate or polyethylene-propylene carbonate), a polyortho-carbonate, a polyamino acid (for example poly-gamma.-L-alanine, poly-.gamma.-benzyl-L-glutamic acid or poly-y-methyl-L-glutamic acid), a hyaluronic acid ester, and the like, and one or more of these polymers can be used.

If the polymers are copolymers, they may be any of random, block and/or graft copolymers. When the above alpha-hydroxycarboxylic acids, hydroxydicarboxylic acids and hydroxytricarboxylic acids have optical activity in their molecules, any one of D-isomers, L-isomers and/or DL-isomers may be used. Among others, alpha-hydroxycarboxylic acid polymer (preferably lactic acid-glycolic acid polymer), its ester, poly-alpha-cyanoacrylic acid esters, etc. may be used, and lactic acid-glycolic acid copolymer (also referred to as poly(lactide-alpha-glycolide) or poly(lactic-co-glycolic acid), and hereinafter referred to as PLGA) are preferred. Thus, in one aspect the polymer useful for the polymeric matrix is PLGA. As used herein, the term PLGA includes polymers of lactic acid (also referred to as polylactide, poly(lactic acid), or PLA). Most preferably, the polymer is the biodegradable poly(d,l-lactide-co-glycolide) polymer.

In a preferred embodiment, the polymeric matrix of the invention is a biocompatible and biodegradable polymeric material. The term “biocompatible” is defined as a polymeric material that is not toxic, is not carcinogenic, and does not significantly induce inflammation in body tissues. The matrix material should be biodegradable wherein the polymeric material should degrade by bodily processes to products readily disposable by the body and should not accumulate in the body. The products of the biodegradation should also be biocompatible with the body in that the polymeric matrix is biocompatible with the body. Particular useful examples of polymeric matrix materials include poly(glycolic acid), poly-D,L-lactic acid, poly-L-lactic acid, copolymers of the foregoing, poly(aliphatic carboxylic acids), copolyoxalates, polycaprolactone, polydioxanone, poly(ortho carbonates), poly(acetals), poly(lactic acid-caprolactone), polyorthoesters, poly(glycolic acid-caprolactone), polyanhydrides, and natural polymers including albumin, casein, and waxes, such as, glycerol mono- and distearate, and the like. The preferred polymer for use in the practice of this invention is dl(polylactide-co-glycolide). It is preferred that the molar ratio of lactide to glycolide in such a copolymer be in the range of from about 75:25 to 50:50.

Useful PLGA polymers may have a weight-average molecular weight of from about 5,000 to 500,000 Daltons, preferably about 150,000 Daltons. Dependent on the rate of degradation to be achieved, different molecular weight of polymers may be used. For a diffusional mechanism of drug release, the polymer should remain intact until all of the drug is released from the polymeric matrix and then degrade. The drug can also be released from the polymeric matrix as the polymeric excipient bioerodes.

The PLGA may be prepared by any conventional method, or may be commercially available. For example, PLGA can be produced by ring-opening polymerization with a suitable catalyst from cyclic lactide, glycolide, etc. (see EP-0058481B2; Effects of polymerization variables on PLGA properties: molecular weight, composition and chain structure).

It is believed that PLGA is biodegradable by means of the degradation of the entire solid polymer composition, due to the break-down of hydrolysable and enzymatically cleavable ester linkages under biological conditions (for example in the presence of water and biological enzymes found in tissues of warm-blooded animals such as humans) to form lactic acid and glycolic acid. Both lactic acid and glycolic acid are water-soluble, non-toxic products of normal metabolism, which may further biodegrade to form carbon dioxide and water. In other words, PLGA is believed to degrade by means of hydrolysis of its ester groups in the presence of water, for example in the body of a warm-blooded animal such as man, to produce lactic acid and glycolic acid and create the acidic microclimate. Lactic and glycolic acid are by-products of various metabolic pathways in the body of a warm-blooded animal such as man under normal physiological conditions and therefore are well tolerated and produce minimal systemic toxicity.

In another embodiment, the polymeric matrix useful for the invention may comprise a star polymer wherein the structure of the polyester is star-shaped. These polyesters have a single polyol residue as a central moiety surrounded by acid residue chains. The polyol moiety may be, e.g., glucose or, e.g., mannitol. These esters are known and described in GB 2,145,422 and in U.S. Pat. No. 5,538,739, the contents of which are incorporated by reference.

The star polymers may be prepared using polyhydroxy compounds, e.g., polyol, e.g., glucose or mannitol as the initiator. The polyol contains at least 3 hydroxy groups and has a molecular weight of up to about 20,000 Daltons, with at least 1, preferably at least 2, e.g., as a mean 3 of the hydroxy groups of the polyol being in the form of ester groups, which contain polylactide or co-polylactide chains. The branched polyesters, e.g., poly(d,l-lactide-co-glycolide) have a central glucose moiety having rays of linear polylactide chains.

The depot compositions of the invention (long-acting injectable compositions having a Compound of the Invention in a polymeric matrix) as hereinbefore described may comprise the polymer in the form of microparticles or nanoparticles, or in a liquid form, with the Compounds of the Invention dispersed or encapsulated therein. “Microparticles” is meant solid particles that contain the Compounds of the Invention either in solution or in solid form wherein such compound is dispersed or dissolved within the polymer that serves as the matrix of the particle. By an appropriate selection of polymeric materials, a microparticle formulation can be made in which the resulting microparticles exhibit both diffusional release and biodegradation release properties.

When the polymer is in the form of microparticles, the microparticles may be prepared using any appropriate method, such as by a solvent evaporation or solvent extraction method. For example, in the solvent evaporation method, the Compounds of the Invention and the polymer may be dissolved in a volatile organic solvent (for example a ketone such as acetone, a halogenated hydrocarbon such as chloroform or methylene chloride, a halogenated aromatic hydrocarbon, a cyclic ether such as dioxane, an ester such as ethyl acetate, a nitrile such as acetonitrile, or an alcohol such as ethanol) and dispersed in an aqueous phase containing a suitable emulsion stabilizer (for example polyvinyl alcohol, PVA). The organic solvent is then evaporated to provide microparticles with the Compounds of the Invention encapsulated therein. In the solvent extraction method, the Compounds of the Invention and polymer may be dissolved in a polar solvent (such as acetonitrile, dichloromethane, methanol, ethyl acetate or methyl formate) and then dispersed in an aqueous phase (such as a water/PVA solution). An emulsion is produced to provide microparticles with the Compounds of the Invention encapsulated therein. Spray drying is an alternative manufacturing technique for preparing the microparticles.

Another method for preparing the microparticles of the invention is also described in both U.S. Pat. Nos. 4,389,330 and 4,530,840.

The microparticle of the present invention can be prepared by any method capable of producing microparticles in a size range acceptable for use in an injectable composition. One preferred method of preparation is that described in U.S. Pat. No. 4,389,330. In this method the active agent is dissolved or dispersed in an appropriate solvent. To the agent-containing medium is added the polymeric matrix material in an amount relative to the active ingredient that provides a product having the desired loading of active agent. Optionally, all of the ingredients of the microparticle product can be blended in the solvent medium together.

Solvents for making such compositions comprising the Compounds of the Invention and the polymeric matrix material that can be employed in the practice of the present invention include organic solvents, such as acetone; halogenated hydrocarbons, such as chloroform, methylene chloride, and the like; aromatic hydrocarbon compounds; halogenated aromatic hydrocarbon compounds; cyclic ethers; alcohols, such as, benzyl alcohol; ethyl acetate; and the like. In one embodiment, the solvent for use in the practice of the present invention may be a mixture of benzyl alcohol and ethyl acetate. Further information for the preparation of microparticles useful for the invention can be found in U.S. Patent Publication Number 2008/0069885, the contents of which are incorporated herein by reference in their entirety.

The amount of the Compounds of the present disclosure incorporated in the microparticles usually ranges from about 1 wt. % to about 90 wt. %, preferably 30 to 50 wt. %, more preferably 35 to 40 wt. %. By weight % is meant parts of the Compounds of the present disclosure per total weight of microparticle.

The pharmaceutical depot compositions may comprise a pharmaceutically-acceptable diluent or carrier, such as a water miscible diluent or carrier.

Details of Osmotic-controlled Release Oral Delivery System composition may be found in EP 1 539 115 (U.S. Pub. No. 2009/0202631) and WO 2000/35419 (US 2001/0036472), the contents of each of which are incorporated by reference in their entirety.

A “therapeutically effective amount” is any amount of the Compounds of the Invention (for example as contained in the pharmaceutical depot) which, when administered to a subject suffering from a disease or disorder, is effective to cause a reduction, remission, or regression of the disease or disorder over the period of time as intended for the treatment.

Dosages employed in practicing the present invention will of course vary depending, e.g., on the particular disease or condition to be treated, the particular Compound of the Invention used, the mode of administration, and the therapy desired. Unless otherwise indicated, an amount of the Compound of the Invention for administration (whether administered as a free base or as a salt form) refers to or is based on the amount of the Compound of the Invention in free base form (i.e., the calculation of the amount is based on the free base amount).

Compounds of the Invention may be administered by any satisfactory mute, including orally, parenterally (intravenously, intramuscular or subcutaneous) or transdermally. In certain embodiments, the Compounds of the Invention, e.g., in depot formulation, is preferably administered parenterally, e.g., by injection, for example, intramuscular or subcutaneous injection.

In general, satisfactory results for the methods of treatment disclosed herein, or use of the Compounds of the Invention as hereinbefore described, as set forth above are indicated to be obtained on oral administration at dosages of the order from about 1 mg to 100 mg once daily, preferably 2.5 mg-50 mg. e.g., 2.5 mg, 5 mg, 10 mg, 20 mg, 30 mg, 40 mg or 50 mg, once daily, preferably via oral administration.

For some disease treatments, lower doses are satisfactory, particularly for sleep disorder treatment, such as from about 2.5 mg-5 mg, e.g., 2.5 mg, 3 mg, 4 mg or 5 mg, of a Compound of the Invention, in free or pharmaceutically acceptable salt form, once daily, preferably via oral administration.

Satisfactory results for methods of treatment involving co-administration of a second therapeutic agent may be obtained at doses of less than 100 mg, preferably less than 50 mg, e.g., less than 40 mg, less than 30 mg, less than 20 mg, less than 10 mg, less than 5 mg, less than 2.5 mg, once daily.

For treatment of the disorders disclosed herein wherein the depot composition is used to achieve longer duration of action, the dosages will be higher relative to the shorter action composition, e.g., higher than 1-100 mg, e.g., 25 mg, 50 mg, 100 mg, 500 mg, 1,000 mg, or greater than 1000 mg. Duration of action of the Compounds of the present disclosure may be controlled by manipulation of the polymer composition, i.e., the polymer: drug ratio and microparticle size. Wherein the composition of the invention is a depot composition, administration by injection is preferred.

The pharmaceutically acceptable salts of the Compounds of the present disclosure can be synthesized from the parent compound which contains a basic or acidic moiety by conventional chemical methods. Generally, such salts can be prepared by reacting the free base forms of these compounds with a stoichiometric amount of the appropriate acid in water or in an organic solvent, or in a mixture of the two; generally, non-aqueous media like ether, ethyl acetate, ethanol, isopropanol, or acetonitrile are preferred. Further details for the preparation of these salts, e.g., toluenesulfonic salt in amorphous or crystal form, may be found in U.S. Pat. Nos. 8,309,722, 8.648,077, 9,199,995, and 9,586,960.

Pharmaceutical compositions comprising Compounds of the present disclosure may be prepared using conventional diluents or excipients (an example include, but is not limited to sesame oil) and techniques known in the galenic art. Thus, oral dosage forms may include tablets, capsules, solutions, suspensions and the like.

The term “concurrently” when referring to a therapeutic use means administration of two or more active ingredients to a patient as part of a regimen for the treatment of a disease or disorder, whether the two or more active agents are given at the same or different times or whether given by the same or different routes of administrations. Concurrent administration of the two or more active ingredients may be at different times on the same day, or on different dates or at different frequencies.

The term “simultaneously” when referring to a therapeutic use means administration of two or more active ingredients at or about the same time by the same route of administration.

The term “separately” when referring to a therapeutic use means administration of two or more active ingredients at or about the same time by different route of administration

### Methods of Making the Compounds of the Invention:

The Compound of Formula A, and methods for its synthesis, including the synthesis of intermediates used in the synthetic schemes described below, have been disclosed in, for example, U.S. Pat. No. 10,245,260, and US 2022/0041600, and US 2022/0064166, the contents of which are hereby incorporated by reference in their entireties.

The synthesis of similar fused gamma-carbolines has been disclosed in, for example, U.S. Pat. Nos. 8,309,722, 8,993,572, US 2017/0183350, WO 2018/126140 and WO 2018/126143, the contents of each of which are incorporated by reference in their entireties. Compounds of the present disclosure can be prepared using similar procedures.

Compound of the Invention wherein R¹ is C(O)—O—C(R^{a})(R^{b})(R^{c}), —C(O)—O—CH₂—O—C(R^{a})(R^{b})(R^{c}) or —C(R⁶)(R⁷)—O—C(O)—R⁸, may be preparing using procedures analogous to those disclosed in international application WO 2019/023063.

Other Compounds of the present disclosure came be made by analogous procedures known to those skilled in the art.

Isolation or purification of the diastereomers of the Compounds of the Invention may be achieved by conventional methods known in the art, e.g., column purification, preparative thin layer chromatography, preparative HPLC, crystallization, trituration, simulated moving beds and the like.

Salts of the Compounds of the present disclosure may be prepared as similarly described in U.S. Pat. Nos. 6,548,493; 7,238,690; 6,552,017; 6,713,471; 7,183,282, 8,648,077; 9,199,995; 9,586,860; U.S. RE39680; and U.S. RE39679, the contents of each of which are incorporated by reference in their entirety.

Diastereomers of prepared compounds can be separated by, for example, HPLC using CHIRALPAK® AY-H, 5μ, 30×250 mm at room temperature and eluted with 10% ethanol/90% hexane/0.1% dimethylethylamine. Peaks can be detected at 230 nm to produce 98-99.9% ee of the diastereomer.

### EXAMPLES

The Compound of Formula A, and methods for its synthesis, are described in U.S. Pat. No. 10,245,260, US 2022/0041600, and US 2022/0064166. For example, in US 2022/0041600, the Compound of Formula A is made according to the following scheme:

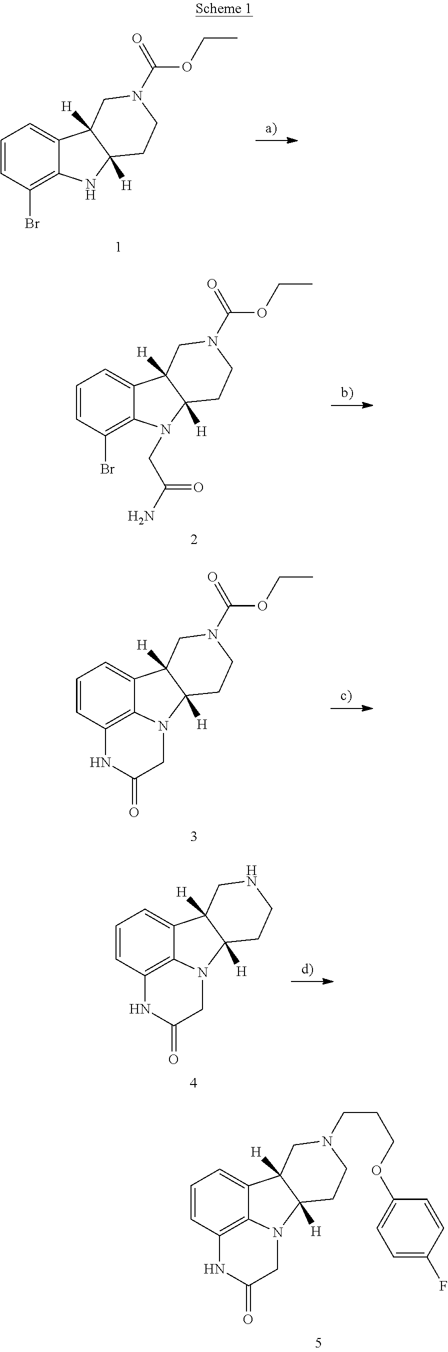 [SMILES: CCOC(=O)N1CC[C@@H]2Nc3c(Br)cccc3[C@@H]2C1 ; CCOC(=O)N1CC[C@H]2[C@@H](C1)c1cccc(Br)c1N2CC(N)=O ; CCOC(=O)N1CC[C@H]2[C@@H](C1)c1cccc3c1N2CC(=O)N3 ; O=C1CN2c3c(cccc3[C@@H]3CNCC[C@@H]32)N1 ; O=C1CN2c3c(cccc3[C@@H]3CN(CCCOc4ccc(F)cc4)CC[C@@H]32)N1]

Compound 1 is disclosed, for example, in U.S. Pat. No. 8,309,722, and US 2020/0102309.

A mixture of ethyl (4aS,9bR)-6-bromo-1,3,4,4a,5,9b-hexahydro-2H-pyrido[4,3-b]indole-2-carboxylate (compound 1, 21.5 g, 66.2 mmol), 2-chloroacetamide (9.3 g, 100 mmol), and KI (17.7 g, 107 mmol) in anhydrous dioxane (60 mL) is heated under an argon atmosphere at 104° C. for 48 h. The reaction is cooled to room temperature and the solvent is removed under reduced pressure. The residue is suspended in dichloromethane (DCM, 200 mL) and washed with water (100 mL). The DCM phase is separated, dried over K₂CO₃ and concentrated to give a brown oil. This oil product is suspended in ethyl acetate (100 mL) and sonicated for 5 min and then allowed to stand at room temperature for 2 h. A precipitate is formed, which is filtered, and the filter cake is rinsed with ethyl acetate (2 mL) and dried under high vacuum. The title compound ethyl (4aS,9bR)-5-(2-amino-2-oxoethyl)-6-bromo-1,3,4,4a,5,9b-hexahydro-2H-pyrido[4,3-b]indole-2-carboxylate (compound 2) is obtained as an off-white solid (19 g, 75% yield), which is directly used in the next reaction without further purification.

A suspension of compound 2 (12.9 g, 33.7 mmol), KI (10.6 g, 63.8 mmol), and CuI (1.34 g, 6.74 mmol) in dioxane (50 mL) is bubbled with argon for 5 min, and then N,N,N′,N′-tetramethylethylenediamine (3 mL) is added. After stirring the reaction mixture at 100° C. for 48 h, the reaction mixture is cooled to room temperature and then poured onto a silica gel pad to filter. The filter cake is rinsed with ethyl acetate (2×250 mL) and the filtrate is concentrated to give the title compound ethyl (6bR,10aS)-2-oxo-2,3,6b,9,10,10a-hexahydro-1H-pyrido[3′,4′:4,5]pyrrolo[1,2,3-de]quinoxaline-8(7H)-carboxylate (compound 3) as a white solid (8 g, 79% yield). This crude product is directly used in the next reaction without further purification.

Compound 3 (6.4 g, 27.9 mmol) is suspended in HBr/acetic acid solution (64 mL, 33% w/w) at room temperature and heated to 50° C. under stirring. After stirring at 50° C. for 8 h, the reaction is cooled to room temperature and ethyl acetate (300 mL) is added. The precipitate is filtered, and the filter cake is washed with ethyl acetate (10 mL) and dried under vacuum. The obtained HBr salt is suspended in methanol (100 mL) and cooled to a temperature of less than 5° C. using an isopropanol/dry ice bath. To this cooled suspension, under stirring, ammonia solution (20 mL, 7 N in methanol) is slowly added until the solution pH is 14. This solution is evaporated to dryness and the residue is dried over high vacuum to give the title compound (6bR,10aS)-6b,7,8,9,10,10a-hexahydro-1H-pyrido[3′,4′:4,5]pyrrolo[1,2,3-de]quinoxalin-2(3H)-one (Compound 4) (7.8 g, >99% yield). This crude product is used directly in the next step without any further purification.

A mixture of compound 4 (1.0 g, 4.4 mmol), 1-(3-chloroproxy)-4-fluorobenzene (1.4 mL, 8.8 mmol) and KI (1.45 g, 8.7 mmol) in DMF (20 mL) is bubbled with argon for 3 min, and then DIPEA (1.50 mL, 8.7 mmol) is added. The mixture is heated to 78° C. and stirred for 2 h, and then cooled to room temperature. The solvent is removed, and the residue is dissolved in DCM (30 mL) and washed with water (20 mL). The DCM phase is dried over K₂CO₃, filtered, and the filtrate is concentrated. The crude product is purified by silica gel column chromatography using a gradient of 0-55% a [ethyl acetate/methanol/7N NH₃ in methanol, 10:1:0.1 v/v/v] in ethyl acetate as eluent. The eluted product is dissolved in methanol (5 mL) and allowed to stand at room temperature for 30 min. A precipitate forms, which is filtered and dried under vacuum to give the final product (6bR,10aS)-8-(3-(4-fluorophenoxy)propyl)-6b,7,8,9,10,10a-hexahydro-1H-pyrido[3′,4′:4,5]pyrrolo[1,2,3-de]quinoxalin-2(3H)-one (compound 5, compound of Formula A) as a white solid (0.63 g, yield: 38%). ¹H NMR (500 MHz, DMSO-d₆) δ 10.3 (s, 1H), 7.2-7.0 (m, 2H), 7.0-6.9 (m, 2H), 6.8-6.7 (m, 1H), 6.6 (t, J=7.55 Hz, 1H), 6.6 (dd, J=1.04, 7.77 Hz, 1H), 4.0 (t, J=6.37 Hz, 2H), 3.8 (d, J=14.53 Hz, 1H), 3.3 (s, 1H), 3.3-3.1 (m, 2H), 2.9-2.8 (m, 1H), 2.7-2.6 (m, 1H), 2.5-2.3 (m, 2H), 2.2-2.0 (m, 1H), 1.9 (dq, J=2.62, 14.64 Hz, 1H), 1.9-1.7 (m, 3H), 1.7 (t, J=10.98 Hz, 1H). HRMS (ESI) m/z calcd for C₂₂H₂₃FN₃O₂ [M+H]⁺: 382.1925; found: 382.1947.

### Example 1: Synthesis of (6bR,10aS)-8-butyl-6b,7,8,9,10,10a-hexahydro-1H-pyrido[3′,4′:4,5]pyrrolo[1,2,3-de]quinoxalin-2(3H)-one

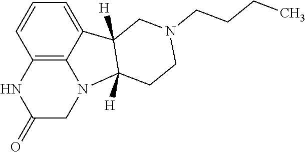 [SMILES: CCCCN1CC[C@H]2[C@@H](C1)c1cccc3c1N2CC(=O)N3]

The compound can be made according to a method analogous to the synthesis of compound 5 in Scheme 1 wherein 1-chlorobutane is added in step d instead of 1-(3-chloropropoxy)-4-fluorobenzene. ¹H NMR (500 MHz, DMSO-d₆) δ 10.34 (s, 1H), 6.77 (dd, J=7.4, 1.0 Hz, 1H), 6.64 (t, J=7.5 Hz, 1H), 6.58 (dd, J=7.9, 1.0 Hz, 1H), 3.80 (d, J=14.5 Hz, 1H), 3.30-3.22 (m, 2H), 3.20 (dt, J=10.7, 6.4 Hz, 1H), 2.83 (ddd, J=11.4, 6.3, 1.8 Hz, 1H), 2.61 (ddd, J=13.3, 4.7, 2.3 Hz, 1H), 2.30-2.13 (m, 2H), 2.11-2.00 (m, 1H), 1.93 (dq, J=14.4, 2.6 Hz, 1H), 1.78 (ddt, J=12.2, 9.6, 4.6 Hz, 1H), 1.63 (t, J=11.0 Hz, 1H), 1.46-1.34 (m, 2H), 1.33-1.22 (m, 2H), 0.87 (t, J=7.3 Hz, 3H). HRMS (ESI) m/z calcd for C₁₇H₂₄N₃O [M+H]⁺: 286.1914.

### Example 2: Synthesis of (6bR,10aS)-8-(3-hydroxypropyl)-6b,7,8,9,10,10a-hexahydro-1H-pyrido[3′,4′:4,5]pyrrolo[1,2,3-de]quinoxalin-2(3H)-one

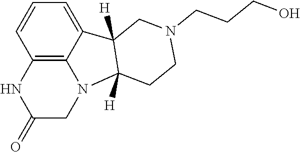 [SMILES: O=C1CN2c3c(cccc3[C@@H]3CN(CCCO)CC[C@@H]32)N1]

The compound can be made according to a method analogous to the synthesis of compound 5 in Scheme 1 wherein 3-chloro-1-propanol is added in step d instead of 1-(3-chloropropoxy)-4-fluorobenzene. ¹H NMR (500 MHz, DMSO-d₆) δ 10.37 (s, 1H), 8.17 (s, 1H), 6.81-6.74 (m, 1H), 6.64 (t, J=7.6 Hz, 1H), 6.58 (dd, J=7.8, 1.0 Hz, 1H), 3.81 (d, J=14.5 Hz, 1H), 3.43 (t, J=6.3 Hz, 2H), 3.31 (d, J=14.4 Hz, 1H), 3.28-3.10 (m, 2H), 2.90 (m, 1H), 2.74-2.62 (m, 1H), 2.44-2.24 (m, 2H), 2.19-2.08 (m, 1H), 2.01-1.87 (m, 1H), 1.86-1.74 (m, 1H), 1.70 (t, J=11.0 Hz, 1H), 1.59 (p, J=6.8 Hz, 2H). HRMS (ESI) m/z calcd for C₁₆H22N3O2 [M+H]⁺: 288.1707; found: 288.1722.

### Example 3: Synthesis of (6bR,10aS)-8-(3-phenoxypropyl)-6b,7,8,9,10,10a-hexahydro-1H-pyrido[3′,4′:4,5]-pyrrolo[1,2,3-de]quinoxalin-2(3H)-one

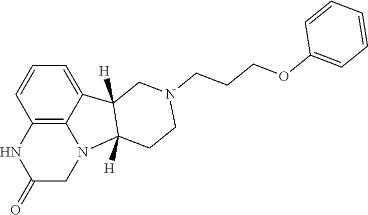 [SMILES: O=C1CN2c3c(cccc3[C@@H]3CN(CCCOc4ccccc4)CC[C@@H]32)N1]

The compound can be made according to a method analogous to the synthesis of compound 5 in Scheme 1 wherein (3-chloropropoxy)benzene is added in step d instead of 1-(3-chloropropoxy)-4-fluorobenzene. ¹H NMR (400 MHz, DMSO-d6) δ 10.33 (s, 1H), 8.14 (d, J=3.5 Hz, 1H), 7.38-7.19 (m, 2H), 6.91 (dd, J=7.9, 4.4 Hz, 2H), 6.78 (d, J=7.1 Hz, 1H), 6.61 (dd.J=19.0, 7.4 Hz, 2H), 4.15-3.92 (m, 2H), 3.81 (d, J=14.5 Hz, 1H), 3.53-3.05 (m, 5H), 2.99-2.83 (m, 1H), 2.66 (s, 1H), 2.45-2.31 (m, 1H), 2.24-2.05 (m, 1H), 2.03-1.77 (m, 3H), 1.72 (t, J=10.8 Hz, 1H). HRMS (ESI) m/z calcd for C₂₂H26N3O2 [M+H]+ 364.2020; found: 364.1970.

### Example 4: Synthesis of (6bR,10aS)-8-(4-(5-chlorothiophen-2-yl)-4-oxobutyl)-6b,7,8,9,10,10a-hexahydro-1H-pyrido[3′,4′:4,5]-pyrrolo[1,2,3-de]quinoxalin-2(3H)-one

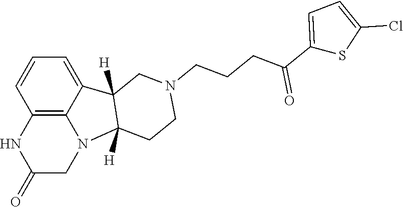 [SMILES: O=C1CN2c3c(cccc3[C@@H]3CN(CCCC(=O)c4ccc(Cl)s4)CC[C@@H]32)N1]

The compound can be made according to a method analogous to the synthesis of compound 5 in Scheme 1 wherein 4-chloro-1-(5-chlorothiophen-2-yl)butan-1-one is added in step d instead of 1-(3-chloropropoxy)-4-fluorobenzene. ¹H NMR (500 MHz, DMSO-d₆) δ 10.35 (s, 1H), 7.85 (d, J=4.1 Hz, 1H), 7.30 (d, J=4.1 Hz, 1H), 6.74 (d, J=7.3 Hz, 1H), 6.63 (t, J=7.5 Hz, 1H), 6.56 (d, J=7.8 Hz, 1H), 3.77 (d, J=14.5 Hz, 1H), 3.28 (d, J=14.4 Hz, 1H), 3.17 (q, J=3.9, 2.1 Hz, 1H), 3.02 (dt, J=11.0, 6.5 Hz, 1H), 2.88 (t, J=6.8 Hz, 2H), 2.76 (dd, J=11.3, 6.5 Hz, 1H), 2.54 (d, J=11.6 Hz, 1H), 2.25 (dp, J=25.8, 6.3 Hz, 2H), 2.10-1.96 (m, 1H), 1.94-1.83 (m, 1H), 1.78 (td, J=12.0, 10.5, 5.4 Hz, 2H), 1.67-1.48 (m, 2H). HRMS (ESI) m/z calcd for C₂₁H₂₃ClN₃O₂S [M+H]⁺: 416.1194.

### Example 5: Synthesis of (6bR,10aS)-8-(3-(6-fluoro-1-methyl-1H-indazol-3-yl)propyl)-6b,7,8,9,10,10a-hexahydro-1H-pyrido-[3′,4′:4,5]pyrrolo[1,2,3-de]quinoxalin-2(3H)-one

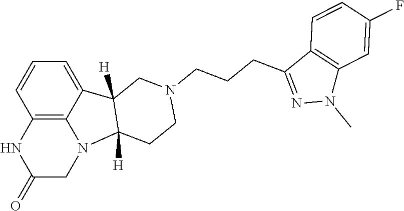 [SMILES: Cn1nc(CCCN2CC[C@H]3[C@@H](C2)c2cccc4c2N3CC(=O)N4)c2ccc(F)cc21]

The compound can be made according to a method analogous to the synthesis of compound 5 in Scheme 1 wherein 3-(3-chloropropyl)-6-fluoro-1-methyl-1H-indazole is added in step d instead of 1-(3-chloropropoxy)-4-fluorobenzene. ¹H NMR (500 MHz, DMSO-d₆) δ 10.36 (s, 1H), 8.16 (s, 1H), 7.76 (dd, J=8.8, 5.2 Hz, 1H), 7.43 (dd, J=10.1, 2.2 Hz, 1H), 6.96 (td, J=9.1, 2.2 Hz, 1H), 6.77 (dd, J=7.4, 1.0 Hz, 1H), 6.63 (t, J=7.5 Hz, 1H), 6.57 (dd, J=7.9, 1.0 Hz, 1H), 3.92 (s, 3H), 3.80 (d, J=14.5 Hz, 1H), 3.30 (d, J=14.4 Hz, 1H), 3.28-3.16 (m, 2H), 2.93-2.82 (m, 3H), 2.65 (d, J=11.8 Hz, 1H), 2.34 (dddd, J=19.4, 12.4, 7.5, 5.3 Hz, 2H), 2.11 (td,J=11.9, 2.8 Hz, 1H), 2.03-1.74 (m, 5H), 1.68 (t, J=11.0 Hz, 1H). HRMS (ESI) m/z calcd for C₂₄H₂₇FN₅O [M+H]⁺: 420.2194.

### Example 6: Synthesis of (6bR,10aS)-8-(3-(4-hydroxyphenoxy)propyl)-6b,7,8,9,10,10a-hexahydro-1H-pyrido[3′,4′:4,5]pyrrolo[1,2,3-de]quinoxalin-2(3H)-one

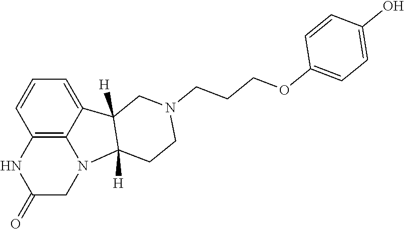 [SMILES: O=C1CN2c3c(cccc3[C@@H]3CN(CCCOc4ccc(O)cc4)CC[C@@H]32)N1]

The compound can be made according to a method analogous to the synthesis of compound 5 in Scheme 1 wherein 4-(3-chloropropoxy)phenol is added in step d instead of 1-(3-chloropropoxy)-4-fluorobenzene. ¹H NMR (500 MHz, DMSO-d₆) δ 10.33 (s, 1H), 8.17 (s, 1H), 6.80-6.75 (m, 1H), 6.75-6.69 (m, 2H), 6.68-6.60 (m, 3H), 6.57 (dd, J=7.8, 1.0 Hz, 1H), 3.88 (t, J=6.4 Hz, 2H), 3.80 (d, J=14.6 Hz, 1H), 3.27-3.15 (m, 3H), 2.86 (dt, J=10.4, 5.0 Hz, 1H), 2.69-2.59 (m, 1H), 2.46-2.29 (m, 2H), 2.09 (t, J=12.0 Hz, 1H), 1.94 (d, J=13.8 Hz, 1H), 1.81 (p, J=6.4 Hz, 3H), 1.67 (t, J=10.7 Hz, 1H). HRMS (ESI) m/z calcd for C₂₂H₂₆FN₃O₃ [M+H]⁺: 380.1969.

### Example 7: Synthesis of (6bR,10aS)-8-(3-(4-(benzyloxy)phenoxy)propyl)-6b,7,8,9,10,10a-hexahydro-1H-pyrido[3′,4′:4,5]-pyrrolo[1,2,3-de]quinoxalin-2(3H)-one

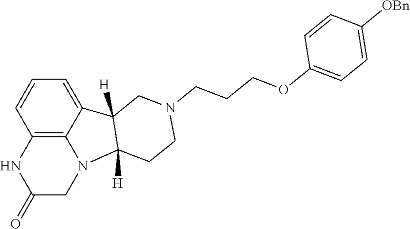 [SMILES: O=C1CN2c3c(cccc3[C@@H]3CN(CCCOc4ccc(OCc5ccccc5)cc4)CC[C@@H]32)N1]

The compound can be made according to a method analogous to the synthesis of compound 5 in Scheme 1 wherein 1-(benzyloxy)-4-(3-chloropropoxy)benzene is added in step d instead of 1-(3-chloropropoxy)-4-fluorobenzene. ¹H NMR (500 MHz, DMSO-d₆) δ 10.34 (s, 1H), 8.16 (d, J=5.8 Hz, 1H), 7.46-7.41 (m, 2H), 7.38 (dd, J=8.4, 6.7 Hz, 2H), 7.35-7.29 (m, 1H), 6.96-6.90 (m, 2H), 6.89-6.82 (m, 2H), 6.78 (d, J=7.2 Hz, 1H), 6.64 (t, J=7.5 Hz, 1H), 6.61-6.56 (m, 1H), 5.03 (s, 2H), 3.93 (t, J=6.4 Hz, 2H), 3.81 (d, J=14.5 Hz, 1H), 3.28-3.19 (m, 3H), 2.89 (d, J=8.4 Hz, 1H), 2.64 (t, J=1.9 Hz, 1H), 2.48-2.31 (m, 2H), 2.09 (d, J=10.7 Hz, 1H), 1.95 (d, J=14.5 Hz, 1H), 1.89-1.75 (m, 3H), 1.69 (d, J=11.6 Hz, 1H). HRMS (ESI) m/z calcd for C₂₉H₃₂N₃O₃ [M+H]⁺: 470.2438.

### Example 8: Synthesis of 5-fluoro-2-(3-((6bR,10aS)-2-oxo-2,3,6b,9,10,10a-hexahydro-1H-pyrido[3′,4′:4,5]pyrrolo[1,2,3-de]quinoxalin-8(7H)-yl)propoxy)benzamide

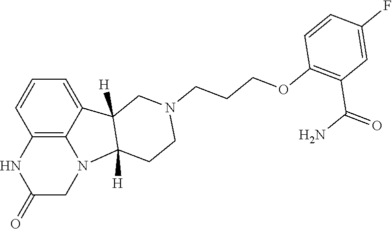 [SMILES: NC(=O)c1cc(F)ccc1OCCCN1CC[C@H]2[C@@H](C1)c1cccc3c1N2CC(=O)N3]

The compound can be made according to a method analogous to the synthesis of compound 5 in Scheme 1 wherein 2-(3-chloropropoxy)-5-fluorobenzamide is added in step d instead of 1-(3-chloropropoxy)-4-fluorobenzene. ¹H NMR (500 MHz, DMSO-d₆) δ 10.36 (s, 1H), 7.72 (d, J=18.6 Hz, 2H), 7.53 (dd, J=9.4, 3.4 Hz, 1H), 7.31 (ddd, J=9.1, 7.7, 3.4 Hz, 1H), 7.17 (dd, J=9.2, 4.3 Hz, 1H), 6.82-6.71 (m, 1H), 6.63 (t, J=7.5 Hz, 1H), 6.57 (dd, J=7.8, 1.0 Hz, 1H), 4.14 (t, J=6.2 Hz, 2H), 3.80 (d, J=14.5 Hz, 1H), 3.30 (d, J=14.5 Hz, 1H), 3.27-3.17 (m, 2H), 2.86 (ddd, J=11.4, 6.4, 1.8 Hz, 1H), 2.70-2.60 (m, 1H), 2.45-2.30 (m, 2H), 2.07 (td, J=11.8, 2.8 Hz, 1H), 1.93 (tt, J=8.7, 4.8 Hz, 3H), 1.85-1.73 (m, 1H), 1.65 (t, J=11.0 Hz, 1H). HRMS (ESI) m/z calcd for C₂₃H₂₆FN₄O₃ [M+H]⁺: 425.1984.

### Example 9: Synthesis of (6bR,10aS)-8-(3-(3,4-difluorophenoxy)propyl)-6b,7,8,9,10,10a-hexahydro-1H-pyrido[3′,4′:4,5]pyrrolo-[1,2,3-de]quinoxalin-2(3H)-one

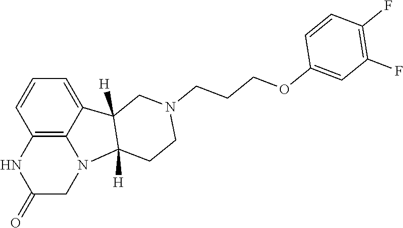 [SMILES: O=C1CN2c3c(cccc3[C@@H]3CN(CCCOc4ccc(F)c(F)c4)CC[C@@H]32)N1]

The compound can be made according to a method analogous to the synthesis of compound 5 in Scheme 1 wherein 4-(3-chloropropoxy)-1,2-difluorobenzene is added in step d instead of 1-(3-chloropropoxy)-4-fluorobenzene. 47% isolated yield. ¹H NMR (400 MHz, DMSO-d₆) δ 10.32 (s, 1H), 7.39-7.23 (m, 1H), 7.11-6.99 (m, 1H), 6.82-6.71 (m, 2H), 6.68-6.54 (m, 2H), 4.00 (t, J=6.3 Hz, 2H), 3.80 (d, J=14.5 Hz, 1H), 3.23-3.17 (m, 1H), 2.90-2.81 (m, 1H), 2.70-2.59 (m, 1H), 2.50 (t, J=1.8 Hz, 2H), 2.45-2.29 (m, 2H), 2.17-2.05 (m, 1H), 1.99-1.91 (m, 1H), 1.90-1.76 (m, 3H), 1.68 (t, J=10.9 Hz, 1H), 1.24 (d, J=2.0 Hz, 1H). HRMS (ESI) m/z calcd for C₂₂H₂₄F₂N₃O₂ [M+H]⁺, 400.1831.

### Example 10: Synthesis of (6bR,10aS)-8-(3-(2,4-difluorophenoxy)propyl)-6b,7,8,9,10,10a-hexahydro-1H-pyrido[3′,4′:4,5]pyrrolo-[1,2,3-de]quinoxalin-2(3H)-one

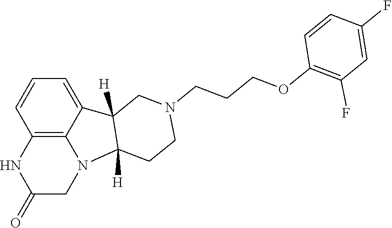 [SMILES: O=C1CN2c3c(cccc3[C@@H]3CN(CCCOc4ccc(F)cc4F)CC[C@@H]32)N1]

The compound can be made according to a method analogous to the synthesis of compound 5 in Scheme 1 wherein 1-(3-chloropropoxy)-2,4-difluorobenzene is added in step d instead of 1-(3-chloropropoxy)-4-fluorobenzene. 30% isolated yield. ¹H NMR (500 MHz, DMSO-d₆) δ 10.47 (s, 1H), 9.15 (s, 1H), 7.46-7.28 (m, 1H), 7.27-7.18 (m, 1H), 7.12-6.97 (m, 1H), 6.94-6.85 (m, 1H), 6.80-6.62 (m, 2H), 4.31-4.06 (m, 2H), 4.02-3.84 (m, 1H), 3.80-3.63 (m, 1H), 3.61-3.34 (m, 3H), 3.28-3.04 (m, 3H), 2.72-2.53 (m, 1H), 2.43-2.29 (m, 1H), 2.28-2.13 (m, 2H), 2.11-2.00 (m, 2H). HRMS (ESI) m/z calcd for C₂₂H₂₄F₂N₃O₂ [M+H]⁺, 400.1831.

### Example 11: Synthesis of (6bR,10aS)-8-(3-(4-fluoro-2,6-dimethylphenoxy)propyl)-6b,7,8,9,10,10a-hexahydro-1H-pyrido[3′,4′:4,5]-pyrrolo[1,2,3-de]quinoxalin-2(3H)-one

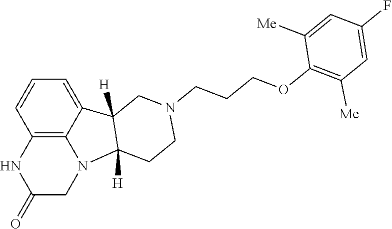 [SMILES: Cc1cc(F)cc(C)c1OCCCN1CC[C@H]2[C@@H](C1)c1cccc3c1N2CC(=O)N3]

The compound can be made according to a method analogous to the synthesis of compound 5 in Scheme 1 wherein 2-(3-chloropropoxy)-5-fluoro-1,3-dimethylbenzene is added in step d instead of 1-(3-chloropropoxy)-4-fluorobenzene. 66% isolated yield. ¹H NMR (400 MHz, DMSO-d₆) δ 10.61 (s, 1H), 7.13 (d, J=9.2 Hz, 2H), 7.05 (dd, J=7.3, 1.1 Hz, 1H), 6.92 (dd, J=7.5 Hz, 1H), 6.87 (dd, J=7.8, 1.1 Hz, 1H), 6.03 (s, 0H), 4.09 (d, J=14.5 Hz, 1H), 4.02 (t, J=6.2 Hz, 2H), 3.64 (s, 0H), 3.58 (s, 3H), 3.53-3.43 (m, 1H), 3.21-3.10 (m, 1H), 2.98-2.87 (m, 1H), 2.81-2.76 (m, 2H), 2.76-2.65 (m, 2H), 2.50 (s, 6H), 2.45-2.36 (m, 1H), 2.28-2.20 (m, 1H), 2.20-2.11 (m, 2H), 2.00 (t, J=11.0 Hz, 1H). HRMS (ESI) m/z calcd for C₂₄H₂₉FN₃O₂ [M+H]⁺, 410.2238.

### Example 12: Synthesis of (6bR,10aS)-8-(3-((4-fluorophenyl)sulfonyl)propyl)-6b,7,8,9,10,10a-hexahydro-1H-pyrido[3′,4′:4,5]pyrrolo-[1,2,3-de]quinoxalin-2(3H)-one

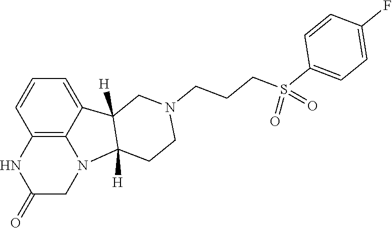 [SMILES: O=C1CN2c3c(cccc3[C@@H]3CN(CCCS(=O)(=O)c4ccc(F)cc4)CC[C@@H]32)N1]

The compound can be made according to a method analogous to the synthesis of compound 5 in Scheme 1 wherein 1-(4-chlorobutyl)-4-fluorobenzene is added in step d instead of 1-(3-chloropropoxy)-4-fluorobenzene. ¹H NMR (400 MHz, DMSO-d₆) δ 10.31 (s, 1H), 8.14 (s, 1H), 8.04-7.93 (m, 2H), 7.52 (dd, J=8.8 Hz, 2H), 6.73 (dd, J=7.4, 1.1 Hz, 1H), 6.63 (dd, J=7.5 Hz, 1H), 6.57 (dd, J=7.8, 1.1 Hz, 1H), 5.75 (s, 0H), 3.78 (d, J=14.5 Hz, 1H), 3.37-3.29 (m, 3H), 3.27 (s, 1H), 3.20-3.11 (m, 1H), 2.72-2.63 (m, 1H), 2.46 (s, 1H), 2.36-2.20 (m, 2H), 2.13-2.01 (m, 1H), 1.96-1.84 (m, 1H), 1.82-1.58 (m, 4H). HRMS (ESI) m/z calcd for C₂₂H₂₅FN₃O₃S [M+H]⁺ 430.1595; found: 430.1513.

### Example 13: Synthesis of (6bR,10aS)-1,1-difluoro-8-(3-(4-fluorophenoxy)propyl)-6b,7,8,9,10,10a-hexahydro-1H-pyrido[3′,4′:4,5]pyrrolo[1,2,3-de]quinoxalin-2(3H)-one

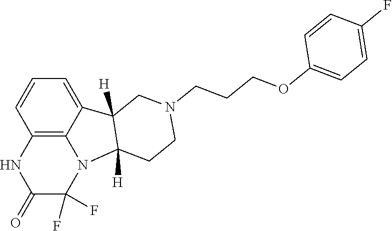 [SMILES: O=C1Nc2cccc3c2N([C@H]2CCN(CCCOc4ccc(F)cc4)C[C@@H]32)C1(F)F]

The compound can be made according to the following scheme:

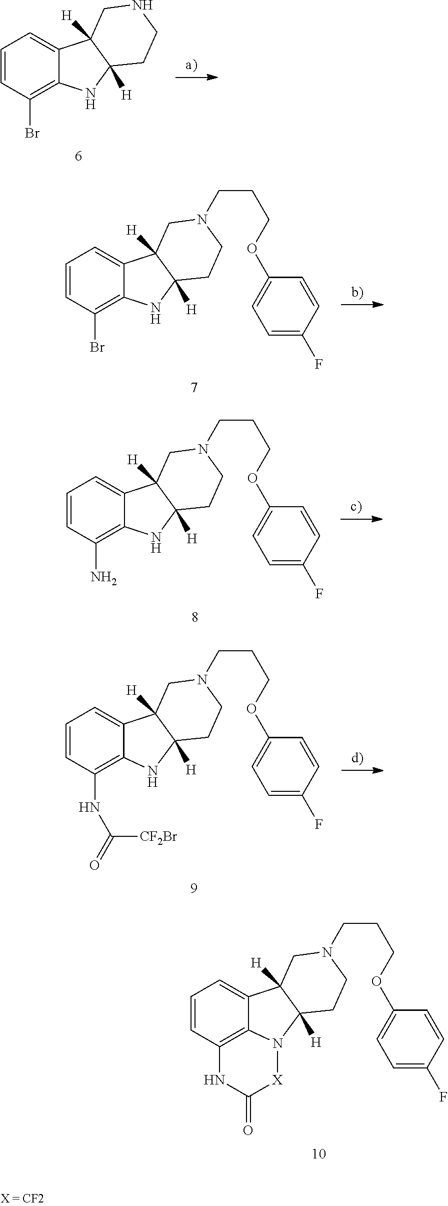

A mixture of compound 6 (5.0 g, 19.7 mmol), 1-(3-chloroproxy)-4-fluorobenzene (3.73 mL, 23.7 mmol) and KI (6.56 g, 39.5 mmol) in DMF (12 mL) is bubbled with argon for 3 min. and DIPEA (6.88 mL, 39.5 mmol) is added. The mixture is heated to 80° C. and stirred for 2 h, and then cooled to room temperature. The solvent is removed, and the residue is further purified by flash column chromatography to give (4aS,9bR)-6-bromo-2-(3-(4-fluorophenoxy)propyl)-2,3,4,4a,5,9b-hexahydro-1H-pyrido[4,3-b]indole (compound 7) (7.6 g, 95% yield) as a pale white solid. ¹H NMR (500 MHz, DMSO-d₆) δ 7.17-7.07 (m, 3H), 7.03 (d, J=7.1 Hz, 1H), 6.98-6.87 (m, 2H), 6.51 (t, J=8.1, 1H), 5.55 (d, J=2.4 Hz, 1H), 3.96 (t, J=6.3 Hz, 2H), 3.73 (m, 1H), 3.17 (m, 1H), 2.82 (d, J=79.5 Hz, 1H), 2.65 (m, 1H), 2.45-2.33 (m, 3H), 2.32-2.22 (m, 1H), 2.08 (dd, J=11.5, 8.8 Hz, 1H), 1.92-1.70 (m, 3H). HRMS (ESI) m/z calcd for C₂₀H₂₃BrFN₂O [M+H]⁺: 405.0972; found: 405.0992.

N,N-dimethylacetamide (6.0 mL) is added to a mixture of copper iodide (0.376 g, 1.97 mmol), cesium carbonate (6.43 g, 19.7 mmol), and compound 7 (4.0 g, 9.87 mmol) at room temperature, then the reaction mixture is bubbled with argon for 5 minutes and sealed. 2,2,6,6-tetramethyl-3,5-heptanedione (2.06 mL, 9.87 mmol) and ammonium hydroxide solution (7.0 mL, 59.2 mmol) are added via syringe. Then the mixture is reacted under microwave irradiation at 95° C., 20 W for 4.5 hours. After the mixture is cooled to room temperature, the solvent is evaporated and the residue is directly purified by column chromatography on silica gel to afford the desired product (4aS,9bR)-2-(3-(4-fluorophenoxy)propyl)-2,3,4,4a,5,9b-hexahydro-1H-pyrido[4,3-b]indol-6-amine (compound 8). (2.1 g, 62% yield) as a black solid. ¹H NMR (500 MHz, DMSO-d₆) δ 8.19 (d, J=1.8 Hz, 2H), 7.15-7.04 (m, 2H), 6.98-6.88 (m, 2H), 6.40 (d, J=5.6 Hz, 2H), 6.37-6.33 (m, 1H), 3.97 (m, 2H), 3.67 (m, 1H), 3.05-3.00 (m, 1H), 2.69 (m, 1H), 2.59-2.52 (m, 1H), 2.48-2.34 (m, 4H), 2.12 (t, J=11.8, 1H), 1.87 (m, 3H), 1.73 (m, 1H). HRMS (ESI) m/z calcd for C₂₀H₂₅FN₃O [M+H]⁺: 342.1976; found: 342.1984.

To a solution of compound 8 (0.760 g, 2.23 mmol) in DCM (8 ml) at 0° C. is added 2-bromo-2,2-difluoroacetyl chloride (0.474 g, 2.45 mmol), and the mixture is stirred at 0° C. for 1 h. The reaction is quenched with water (6 ml) and extracted with DCM (15 mL). The separated organic phase is washed with water (10 ml) and evaporated to dryness. The obtained product, 2-bromo-2,2-difluoro-N-((4aS,9bR)-2-(3-(4-fluorophenoxy)propyl)-2,3,4,4a,5,9b-hexahydro-1H-pyrido[4,3-b]indol-6-yl)acetamide (compound 9) (0.980 mg, 88% yield), is used directly in the next reaction without further purification. HRMS (ESI) m/z calcd for C₂₂H₂₄BrF₃N₃O₂ [M+H]⁺ 498.0999; found: 498.1027.

A 25 mL round-bottom flask is charged with compound 9 (0.042 g, 0.085 mmol) and toluene (0.8 mL) under argon atmosphere. To this suspension is dropped 1M lithium bis(trimethylsilyl)amide in toluene (0.13 mL, 0.13 mmol) and the mixture is stirred at 0° C. for 2 h, then at room temperature for 16 h. The reaction is cooled to 0° C., quenched with water (0.5 mL) and evaporated to dryness. The residue is purified on HPLC to provide the title compound (6bR,10aS)-1,1-difluoro-8-(3-(4-fluorophenoxy)propyl)-6b,7,8,9,10,10a-hexahydro-1H-pyrido[3′,4′:4,5]pyrrolo[1,2,3-de]quinoxalin-2(3H)-one (compound 10) as an off-white solid (0.007 g, yield 19%). ¹H NMR (400 MHz, DMSO-d₆) δ 10.36 (s, 1H), 8.19 (s, 1H), 7.16-7.03 (m, 2H), 6.96-6.85 (m, 2H), 6.16-5.99 (m, 1H), 5.82 (d, J=3.0 Hz, 1H), 4.00-3.89 (m, 2H), 3.77 (t, J=5.9 Hz, 1H), 3.00-2.85 (m, 2H), 2.74-2.61 (m, 2H), 2.40-2.23 (m, 2H), 2.23-2.10 (m, 1H), 2.07-1.93 (m, 1H), 1.91-1.76 (m, 2H), 1.76-1.64 (m, 1H), 1.54-1.37 (m, 1H). HRMS (ESI) m/z calcd for C₂₁H₂₃FN₃O₂[M+H]⁺ 418.1737; found: 418.2122.

### Example 14: Synthesis of N-((4aS,9bR)-2-(3-(4-fluorophenoxy)propyl)-2,3,4,4a,5,9b-hexahydro-1H-pyrido[4,3-b]indol-6-yl)acetamide

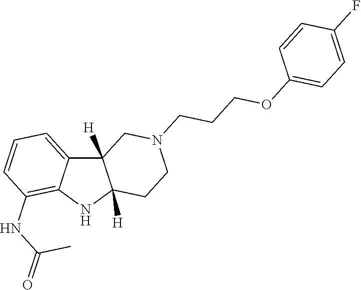 [SMILES: CC(=O)Nc1cccc2c1N[C@H]1CCN(CCCOc3ccc(F)cc3)C[C@@H]21]

N,N-dimethylacetamide (DMA) (10.0 mL) is added to a 100 mL flask containing a mixture of compound 7 (1.13 g, 2.8 mmol), CuI (0.49 g, 2.8 mmol), Cs₂CO₃ (1.8 g, 5.6 mmol), and acetamide (1.65 g, 28 mmol) at room temperature. The mixture is bubbled with argon for 5 min, and then 1,2-dimethylethylenediamine (0.60 mL, 5.6 mmol) is added via syringe. The mixture is stirred at 100° C. for 1 h and then cooled to room temperature. The solvent is evaporated, and the residue is directly purified with semi-preparative HPLC to afford the title compound as a formic acid salt (176 mg, 14% yield). ¹H NMR (500 MHz, DMSO-d₆) δ 9.44 (s, 1H), 7.18-7.03 (m, 3H), 6.94-6.89 (m. 2H), 6.88 (d, J=7.2 Hz, 1H), 6.64-6.50 (m, 1H), 5.15 (s, 1H), 3.96 (t, J=6.3 Hz, 2H), 3.72 (dt, J=7.2, 4.6 Hz, 1H), 3.10 (dt, J=8.5, 6.3 Hz, 1H), 2.68 (dd, J=11.6, 6.0 Hz, 1H), 2.49-2.45 (m, 1H), 2.43 (q, J=6.6 Hz, 2H), 2.34 (td, J=10.8, 3.4 Hz, 1H), 2.13 (dd, J=11.5, 8.7 Hz, 1H), 2.03 (s, 3H), 1.92-1.79 (m, 3H), 1.74 (dq, J=9.5, 4.1 Hz, 1H). HRMS (ESI) m/z calcd for C₂₂H₂₇FN₃O₂ [M+H]⁺: 384.2082.

### Example 15: N-((4aS,9bR)-2-(4-(4-fluorophenyl)-4-oxobutyl)-2,3,4,4a,5,9b-hexahydro-1H-pyrido[4,3-b]indol-6-yl)acetamide

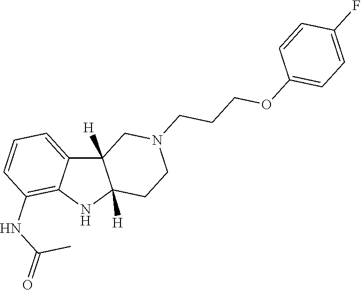 [SMILES: CC(=O)Nc1cccc2c1N[C@H]1CCN(CCCOc3ccc(F)cc3)C[C@@H]21]

4-((4aS,9bR)-6-bromo-1,3,4,4a,5,9b-hexahydro-2H-pyrido[4,3-b]indol-2-yl)-1-(4-fluorophenyl)butan-1-one may be prepared according to the procedure for compound 7 above, but substituting 4-chloro-(4′-fluorobutyrophenone) for 1-(3-chloroproxy)-4-fluorobenzene.

N,N-dimethylacetamide (10.0 mL) is added to a mixture of copper iodide (0.460 g, 2.4 mmol), cesium carbonate (1.56 g, 4.80 mmol), acetamide (1.42 g, 24 mmol) and 4-((4aS,9bR)-6-bromo-1,3,4,4a,5,9b-hexahydro-2H-pyrido[4,3-b]indol-2-yl)-1-(4-fluorophenyl)butan-1-one (1.0 g, 2.40 mmol) at room temperature, then the reaction mixture is bubbled with argon for 5 minutes. 1,2-dimethylethylenediamine (0.52 mL, 4.79 mmol) is added via syringe. The mixture is stirred at 100° C. for 1 hour, and then cooled to room temperature. The solvent is evaporated, and the residue is directly purified by column chromatography on silica gel. The product is purified again by semipreparative HPLC to afford the desired product (0.137 g, 14% yield). ¹H NMR (500 MHz, DMSO-d₆) δ 9.47 (s, 1H), 8.06 (dd, J=8.8, 5.6 Hz, 2H), 7.37 (t, J=8.8 Hz, 2H), 7.22-7.02 (m, 1H), 6.91 (d, J=7.1 Hz, 1H), 6.61 (t, J=7.6 Hz, 1H), 5.17 (s, 1H), 3.72 (dt, J=6.8, 4.5 Hz, 1H), 2.68 (dd, J=11.7, 5.8 Hz, 1H), 2.50 (dt, J=10.2, 4.6 Hz, 1H), 2.37 (tt, J=13.4, 6.9 Hz, 3H), 2.18-2.12 (m, 1H), 2.06 (s, 3H), 1.84 (m, 2H), 1.76 (m, 2H).

### Example 16: Synthesis of N-((4aS,9bR)-2-(4-(4-fluorophenyl)-4-hydroxybutyl)-2,3,4,4a,5,9b-hexahydro-1H-pyrido[4,3-b]indol-6-yl)acetamide

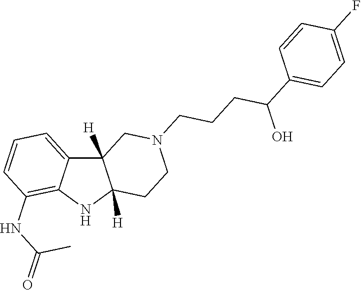 [SMILES: CC(=O)Nc1cccc2c1N[C@H]1CCN(CCCC(O)c3ccc(F)cc3)C[C@@H]21]

Sodium borohydride (0.028 g, 0.76 mmol) is added to a solution of the Compound of Example 15 (0.050 g, 0.126 mmol) in methanol (2 ml). After completion, the reaction mixture is stirred at room temperature for 30 minutes, and then the reaction is quenched with methanol. The solvent is evaporated and the residue is purified by semipreparative HPLC to give the desired product (0.036 g, 72% yield). ¹H NMR (500 MHz, DMSO-d₆) δ 9.45 (s, 1H), 7.47-7.26 (m, 2H), 7.21-6.96 (m, 3H), 6.95-6.76 (m, 1H), 6.59 (t, J=7.6 Hz, 1H), 5.15 (s, 1H), 4.53 (q, J=5.3, 3.7 Hz, 1H), 3.83-3.57 (m, 1H), 3.20-2.88 (m, 1H), 2.78-2.54 (m, 1H), 2.49-2.39 (m, 1H), 2.36-2.23 (m, 3H), 2.15-2.06 (m, 1H), 2.03 (s, 3H), 1.90-1.78 (m, 1H), 1.77-1.67 (m, 1H), 1.63-1.47 (m, 3H), 1.46-1.33 (m, 1H).

### Examples 17-26: Additional Compounds

The following additional compounds are prepared using procedures substantially as described above, by reacting the intermediate compound 4 with the appropriate alkyl halide to provide the target compound in generally good yield:

| Ex. No. | Structure | ¹H NMR (500 MHz, DMSO) δ |
|---|---|---|
| 17 | 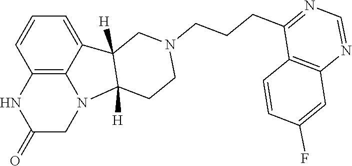 [SMILES: O=C1CN2c3c(cccc3[C@@H]3CN(CCCc4ncnc5cc(F)ccc45)CC[C@@H]32)N1] | 10.34 (s, 1H), 9.16 (s, 1H), 8.49 (dd, J = 9.3, 6.1 Hz, 1H), 7.75 (dd, J = 10.0, 2.7 Hz, 1H), 7.67 (td, J = 8.9, 2.7 Hz, 1H), 6.74 (d, J = 7.3 Hz, 1H), 6.62 (t, J = 7.5 Hz, 1H), 6.56 (dd, J = 7.8, 1.0 Hz, 1H), 3.77 (d, J = 14.6 Hz, 1H), 3.40-3.27 (m, 5H), 3.17 (s, 1H), 3.01 (dt, J = 12.0, 6.4 Hz, 1H), 2.79 (dd, J = 11.4, 6.4 Hz, 1H), 2.56 (d, J = 11.1 Hz, 1H), 2.36 (dtd, J = 25.9, 12.5, 6.6 Hz, 2H), 2.06 (d, J = 11.6 Hz, 1H), 1.98 (dd, J = 8.1, 6.0 Hz, 2H), 1.88 (d, J = 14.7 Hz, 1H), 1.61 (q, J = 13.6 Hz, |
| | | 2H). |
| 18 | 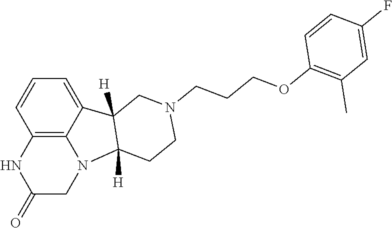 [SMILES: Cc1cc(F)ccc1OCCCN1CC[C@H]2[C@@H](C1)c1cccc3c1N2CC(=O)N3] | 10.40 (s, 1H), 7.05-6.99 (m, 1H), 6.99- 6.92 (m, 1H), 6.90 (dd, J = 9.0, 4.9 Hz, 1H), 6.82 (dd, J = 7.4, 1.0 Hz, 1H), 6.68 (t, J = 7.6 Hz, 1H), 6.65-6.58 (m, 1H), 3.99 (t, J = 6.1 Hz, 2H), 3.87 (d, J = 14.6 Hz, 1H), 3.47- 3.30 (m, 4H), 3.14-3.00 (m, 1H), 2.93- 2.76 (m, 2H), 2.73-2.58 (m, 1H), 2.24- 2.16 (m, 1H), 2.15 (s, 3H), 2.12-2.09 (m, 1H), 2.04-1.98 (m, 2H), 1.97-1.89 (m, 1H). |
| 19 | 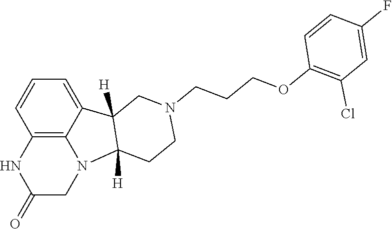 [SMILES: O=C1CN2c3c(cccc3[C@@H]3CN(CCCOc4ccc(F)cc4Cl)CC[C@@H]32)N1] | 10.34 (s, 1H), 7.41 (dt, J = 8.3, 1.7 Hz, 1H), 7.16 (dd, J = 6.7, 1.7 Hz, 2H), 6.77 (dd, J = 7.3, 1.0 Hz, 1H), 6.64 (t, J = 7.5 Hz, 1H), 6.58 (dd, J = 7.8, 1.1 Hz, 1H), 4.07 (t, J = 6.3 Hz, 2H), 3.81 (d, J = 14.5 Hz, 1H), 3.32 (s, 1H), 3.27-3.18 (m, 2H), 3.00-2.85 (m, 1H), 2.76-2.63 (m, 1H), 2.48-2.30 (m, 2H), 2.31-2.09 (m, 1H), 2.04-1.85 (m, 3H), 1.85-1.76 (m, 1H), 1.72 (t, J = 11.0 Hz, 1H). |
| 20 | 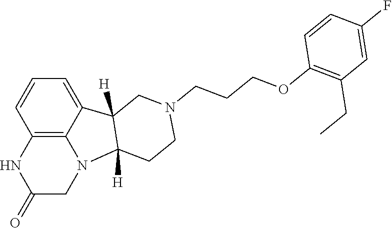 [SMILES: CCc1cc(F)ccc1OCCCN1CC[C@H]2[C@@H](C1)c1cccc3c1N2CC(=O)N3] | 10.35 (s, 1H), 7.02-6.97 (m, 1H), 6.96- 6.86 (m, 2H), 6.77 (dd, J = 7.3, 1.0 Hz, 1H), 6.64 (t, J = 7.5 Hz, 1H), 6.58 (dd, J = 7.8, 1.0 Hz, 1H), 3.97 (t, J = 6.2 Hz, 2H), 3.81 (d, J = 14.6 Hz, 1H), 3.29-3.12 (m, 3H), 3.01- 2.83 (m, 1H), 2.75-2.64 (m, 1H), 2.55 (q, J = 7.5 Hz, 2H), 2.48-2.38 (m, 2H), 2.16 (td, J = 11.8, 2.8 Hz, 1H), 2.03-1.92 (m, 1H), 1.89 (p, J = 6.7 Hz, 2H), 1.86-1.77 (m, 1H), 1.73 (t, J = 10.9 Hz, 1H), 1.12 (t, J = 7.5 Hz, 3H). |
| 21 | 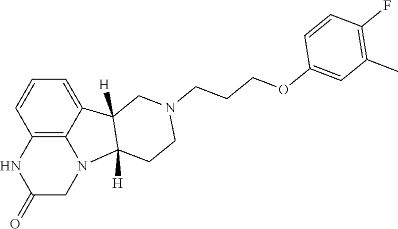 [SMILES: Cc1cc(OCCCN2CC[C@H]3[C@@H](C2)c2cccc4c2N3CC(=O)N4)ccc1F] | N/A |
| 22 | 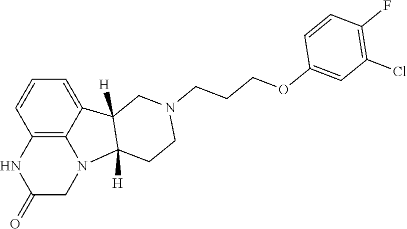 [SMILES: O=C1CN2c3c(cccc3[C@@H]3CN(CCCOc4ccc(F)c(Cl)c4)CC[C@@H]32)N1] | N/A |
| 23 | 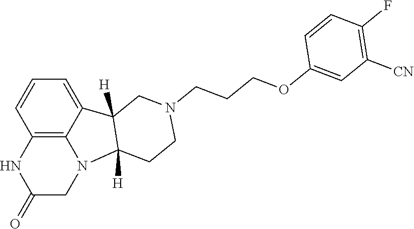 [SMILES: N#Cc1cc(OCCCN2CC[C@H]3[C@@H](C2)c2cccc4c2N3CC(=O)N4)ccc1F] | N/A |
| 24 | 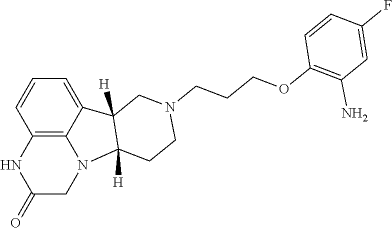 [SMILES: Nc1cc(F)ccc1OCCCN1CC[C@H]2[C@@H](C1)c1cccc3c1N2CC(=O)N3] | 10.35 (s, 1H), 8.15 (s, 1H), 7.86 (dd, J = 9.1, 6.7 Hz, 1H), 7.44 (s, 2H), 6.80-6.72 (m, 1H), 6.63 (t, J = 7.6 Hz, 1H), 6.57 (dd, J = 7.8, 1.0 Hz, 1H), 6.49 (dd, J = 12.0, 2.7 Hz, 1H), 6.35 (td, J = 8.6, 2.7 Hz, 1H), 3.79 (d, J = 14.5 Hz, 1H), 3.29 (d, J = 14.5 Hz, 2H), 3.26-3.12 (m, 2H), 2.90 (t, J = 7.1 Hz, 2H), 2.83 (ddd, J = 11.5, 6.5, 1.9 Hz, 1H), 2.61 (d, J = 11.1 Hz, 1H), 2.41-2.15 (m, 2H), 2.08 (td, J = 11.7, 2.8 Hz, 1H), 1.98-1.86 (m, 1H), 1.85-1.69 (m, 3H), 1.65 (t, J = 11.1 Hz, 1H). |
| 25 | 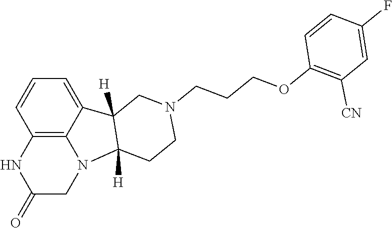 [SMILES: N#Cc1cc(F)ccc1OCCCN1CC[C@H]2[C@@H](C1)c1cccc3c1N2CC(=O)N3] | 10.36 (s, 1H), 7.74 (dd, J = 8.2, 3.2 Hz, 1H), 7.55 (ddd, J = 9.3, 8.3, 3.2 Hz, 1H), 7.28 (dd, J = 9.4, 4.2 Hz, 1H), 6.77 (dd, J= 7.4, 1.0 Hz, 1H), 6.63 (t, J = 7.5 Hz, 1H), 6.57 (dd, J = 7.8, 1.0 Hz, 1H), 4.16 (t, J = 6.2 Hz, 2H), 3.80 (d, J = 14.5 Hz, 1H), 3.30 (d, J = 14.5 Hz, 1H), 3.27-3.16 (m, 2H), 2.95-2.81 (m, 1H), 2.69-2.58 (m, 1H), 2.48-2.29 (m, 2H), 2.15-2.05 (m, 1H), 1.97-1.84 (m, 3H), 1.78 (ddt, J = 12.7, 10.6, 3.9 Hz, 1H), 1.67 (t, J = 11.0 Hz, 1H). |
| 26 | 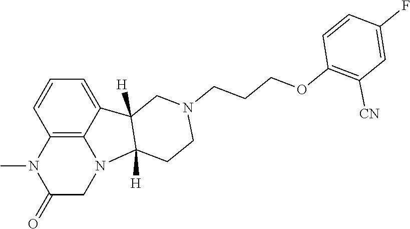 [SMILES: CN1C(=O)CN2c3c(cccc31)[C@@H]1CN(CCCOc3ccc(F)cc3C#N)CC[C@@H]12] | 7.74 (dd, J = 8.2, 3.2 Hz, 1H), 7.55 (ddd, J = 9.3, 8.3, 3.2 Hz, 1H), 7.28 (dd, J = 9.3, 4.2 Hz, 1H), 6.90-6.85 (m, 1H), 6.83 (dd, J = 8.1, 1.0 Hz, 1H), 6.75 (dd, J = 8.0, 7.3 Hz, 1H), 4.16 (t, J = 6.3 Hz, 2H), 3.95 (d, J = 14.4 Hz, 1H), 3.38 (d, J = 14.4 Hz, 1H), 3.25 (t, J = 4.7 Hz, 1H), 3.22 (s, 3H), 2.93-2.83 (m, 1H), 2.68-2.60 (m, 1H), 2.40 (tdd, J = 19.3, 12.7, 6.8 Hz, 2H), 2.09 (td, J = 11.9, 11.3, 2.8 Hz, 1H), 2.00-1.84 (m, 3H), 1.80 (ddt, J = 13.7, 9.2, 4.5 Hz, 1H), 1.65 (t, J = 10.8 Hz, 1H), 1.22-1.14 (m, 1H) |

### Example 27: Receptor Binding Profile

Receptor binding is determined for the Compound of Example 1 (the Compound of Formula A), and the Compounds of Examples 2 to 6. The following literature procedures are used, each of which reference is incorporated herein by reference in their entireties: 5-HT_{2A}: Bryant, H. U. et al. (1996), Life Sci., 15:1259-1268; D2: Hall, D. A. and Strange, P. G. (1997), Brit. J. Pharmacol., 121:731-736; D1: Zhou, Q. Y. et al. (1990), Nature, 347:76-80; SERT: Park, Y. M. et al. (1999), Anal. Biochem., 269:94-104; Mu opioid receptor: Wang, J. B. et al. (1994), FEBS Lett., 338:217-222.

In general, the results are expressed as a percent of control specific binding:

$$\frac{m e a sured specific binding}{control specific binding} \times 100$$

and as a percent inhibition of control specific binding:

$$100 - ( \frac{m e a sured specific binding}{control specific binding} \times 100 )$$

obtained in the presence of the test compounds.

The IC₅₀ values (concentration causing a half-maximal inhibition of control specific binding) and Hill coefficients (nH) are determined by non-linear regression analysis of the competition curves generated with mean replicate values using Hill equation curve fitting:

$$Y = D + [ ( \frac{A - D}{{1 + C / {C}_{50} )}^{nH}} ]$$

where Y=specific binding, A=left asymptote of the curve, D=right asymptote of the curve, C=compound concentration, C₅₀=IC₅₀, and nH=slope factor. This analysis was performed using in-house software and validated by comparison with data generated by the commercial software SigmaPlot® 4.0 for Windows® (© 1997 by SPSS Inc.). The inhibition constants (Ki) were calculated using the Cheng Prusoff equation:

$$Ki = \frac{I {C}_{50}}{( 1 + L / {K}_{D} )}$$

where L=concentration of radioligand in the assay, and K_{D}=affinity of the radioligand for the receptor. A Scatchard plot is used to determine the K_{D}.

The following receptor affinity results are obtained (with the Compound of Formula A for comparison; all percent values are maximum inhibition; other values are Ki):

| | Ki (nM) or maximum inhibition | | | | | | | | |
|---|---|---|---|---|---|---|---|---|---|
| Receptor | Fm. A | Ex. 1 | Ex. 2 | Ex. 3 | Ex. 4 | Ex. 5 | Ex. 6 | Ex. 7 | Ex. 8 |
| SERT(at 200 nM) | 590 (15%) | NI | NI | 8%^{a} | 66 | 35% | 11% | 68% | 9.6% |
| 5-HT_{2A} (at 100 nM) | 8.3 (98%) | NI | 8% | 2.84 | 18.3 | 12.4 | 77% | 76% | 14% |
| D2 (at 100 nM) | 160(26%) | 15% | NI | 34% | 41% | 43% | 41% | 23% | NI^{b} |
| D1 (at 100 nM) | 50 (44%) | NI | NI | 24% | 34% | 54% | 0.86% | 4.6% | 0.78% |
| Mu (at 100 nM) | 11 (86%) | NI | 11% | 19.7 | 8.15 | 7.1 | 13% | 56% | 76% |

^{a}at 50 μM;
^{b}at 100 μM;
^{c}at 200 μM;

| | Ki (nM) or maximum inhibition | | | | | | | |
|---|---|---|---|---|---|---|---|---|
| Receptor | Ex. 9 | Ex. 10 | Ex. 11 | Ex.12 | Ex. 13 | Ex. 14 | Ex. 15 | Ex. 16 |
| SERT (at 200 nM) | 15%^{c} | NI^{c} | 34%^{c} | NI | 6% | 4% | NI | NI |
| 5-HT_{2A} (at 100 nM) | 16.1 | 15.2 | 40% | 33% | 21% | 67% | 80% | NI |
| D2 (at 100 nM) | 7% | 36% | NI | NI | 7% | 11% | 49% | NI |
| D1 (at 100 nM) | 15% | 21% | NI | NI | 24% | 25% | 45% | NI |
| Mu (at 100 nM) | 1.83 | 61% | 4.35 | NI | NI | 9% | 11% | NI |

^{a}at 50 μM;
^{b}at 100 μM;
^{c}at 200 μM;

| | Ki (nM) or maximum inhibition | | | | | | | |
|---|---|---|---|---|---|---|---|---|
| Receptor | Fm. A | Ex. 17 | Ex. 18 | Ex. 19 | Ex. 20 | Ex. 21 | Ex. 22 | Ex. 23 |
| SERT (% at 200 nM) | 11% | Tbd | 32% | 23% | 26% | — | — | — |
| SERT (% at 100 nM) | | — | — | — | — | 14% | 6% | 13% |
| 5-HT_{2A} (at 100 nM) | 8.3 | Tbd | 0.4 | 0.6 | 0.3 | 77% | 65% | 35% |
| D2 (% at 100 nM) | 38% | Tbd | 69% | 80% | 69% | 0% | 0% | 0% |
| D1 (% at 100 nM) | 28% | Tbd | 40% | 33% | 66% | <1% | 0% | 0% |
| Mu | 11 | Tbd | 2.9 | 3.0 | 5.4 | 69 | 74 | 65 |

| | Ki (nM) or maximum inhibition | | | | |
|---|---|---|---|---|---|
| Receptor | Fm. A | | Ex. 24 | Ex. 25 | Ex. 26 |
| SERT(% at 200 nM) | 590 | (7%) | 60 | 35 | 93% |
| 5-HT_{2A} (% at 100 nM) | 8.3 | (90%) | 0.34 | 3.8 | 91% |
| D2 (% at 100 nM) | 160 | (26%) | 1.0 | 43 | 14% |
| D1 (% at 100 nM) | 50 | (31%) | 16 | 74 | 93% |
| Mu (Ki, nM) | 11 | (76%) | 4.1 | 8.4 | 5% |

The forgoing examples are merely exemplary and are not meant to limit the scope of the present disclosure in any way.

1. The compound according to claim 27, wherein the compound is a compound of a Formula I; 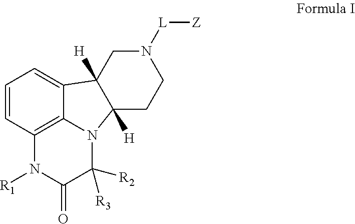
wherein:
R¹ is H, C₁₋₆alkyl, —C(O)—O—C(R^{a})(R^{b})(R^{c}), —C(O)—O—CH₂—O—C(R^{a})(R^{b})(R^{c}) or —C(R⁶)(R⁷)—O—C(O)—R⁸;
R² and R³ are independently selected from H, D, C₁₋₆alkyl (e.g., methyl), C₁₋₆alkoxy (e.g., methoxy), halo (e.g., F), cyano, or hydroxy;
or wherein R² and R³ and the carbon to which they are attached collectively form a group —CH₂CH₂—, or
wherein R² and R³ and the carbon to which they are attached are absent;
L is C₁₋₆alkylene (e.g., ethylene, propylene, or butylene), C₁₋₆alkoxy (e.g., propoxy or butoxy), C₂₋₃alkoxyC₁₋₃alkylene (e.g., CH₂CH₂OCH₂), C₁₋₆alkylamino or N—C₁₋₆alkyl C₁₋₆alkylamino (e.g., propylamino or N-methylpropylamino), C₁₋₆alkylthio (e.g., —CH₂CH₂CH₂S—), C₁₋₆alkylsulfonyl (e.g., —CH₂CH₂CH₂S(O)₂—), or —C₁₋₆alkyl-C(O)— (e.g., 4-butanoyl), each of which is optionally substituted with one or more R⁴ moieties;
each R⁴ is independently selected from C₁₋₆alkyl (e.g., methyl), C₁₋₆alkoxy (e.g., methoxy), halo (e.g., F), cyano, hydroxy, amino (—NH₂), C₁₋₆alkylaryl (e.g., benzyl), C₁₋₆alkoxyaryl (e.g., benzyloxy), aryloxy (e.g., phenoxy), —C(O)-aryl, —C(O)—C₁₋₆alkyl, —C(O)OH, —C(O)NH₂, —C(O)NH(C₁₋₆alkyl), and C(O)N(C₁₋₆alkyl)(C₁₋₆alkyl);
Z is selected from aryl (e.g., phenyl) and heteroaryl (e.g., thiophenyl, furanyl, pyridyl, indazolyl, benzimidazolyl, benzisoxazolyl), methyl or OH, each optionally substituted with one or more R⁴ moieties, optionally wherein Z is unsubstituted;
R⁸ is —C(R^{a})(R^{b})(R^{c}), —O—C(R^{a})(R^{b})(R^{c}), or —N(R^{d})(R^{e});
R^{a}, R^{b} and R^{c} are each independently selected from H and C₁₋₂₄alkyl;
R^{d} and R^{e} are each independently selected from H and C₁₋₂₄alkyl;
R⁶ and R⁷ are each independently selected from H, C₁₋₆alkyl, carboxy and C₁₋₆alkoxycarbonyl;
provided that if Z is aryl or heteroaryl at least one group R⁴ on substituent Z is selected from amino (—NH₂), C₁₋₆alkylaryl (e.g., benzyl), C₁₋₆alkoxyaryl (e.g., benzyloxy), aryloxy (e.g., phenoxy), —C(O)-aryl, —C(O)—C₁₋₆alkyl, —C(O)OH, —C(O)NH₂, —C(O)NH(C₁₋₆alkyl), and C(O)N(C₁₋₆alkyl)(C₁₋₆alkyl);
in free or salt form (e.g., pharmaceutically acceptable salt form), for example in an isolated or purified free or salt form (e.g., pharmaceutically acceptable salt form).

2. The compound according to claim 1, wherein R¹, R² and R³ are H.

3. The compound according to claim 1, wherein L is —(CH₂)ₙ—X—, and wherein n is an integer selected from 2, 3 and 4, and X is selected from —O—, —S—, —NH—, —N(C₁₋₆alkyl)-, —CH₂—, and —C(O)—.

4. The compound according to claim 1, wherein Z is phenyl substituted with one, two, three or four R⁴ moieties, and wherein the one, two three or four R⁴ moieties are independently selected from methyl, methoxy, hydroxy, amino (—NH₂), halo (e.g., fluoro, chloro, bromo or iodo), cyano, benzyl, benzyloxy, phenoxy, benzoyl, acetyl, pivaloyl, —C(O)OH, —C(O)NH₂, —C(O)NH(CH₃), and C(O)N(CH₃)(CH₃).

5. The compound according to claim 1, wherein the compound is selected from the group consisting of: 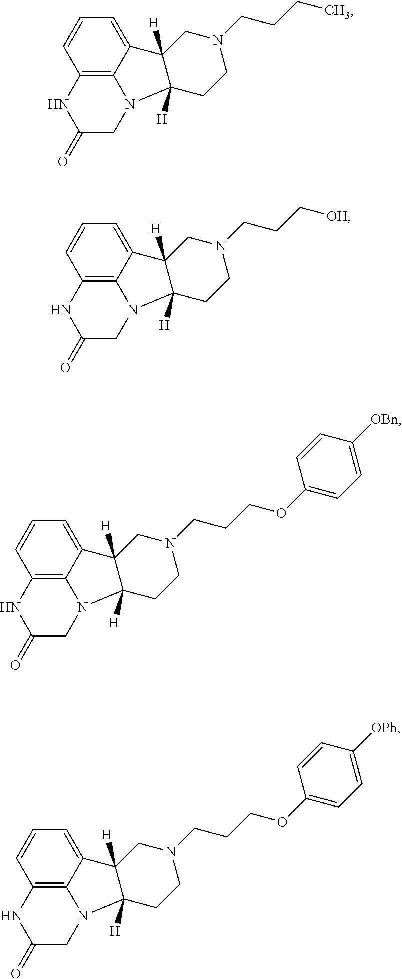 [SMILES: CCCCN1CC[C@H]2[C@@H](C1)c1cccc3c1N2CC(=O)N3 ; O=C1CN2c3c(cccc3[C@@H]3CN(CCCO)CC[C@@H]32)N1 ; O=C1CN2c3c(cccc3[C@@H]3CN(CCCOc4ccc(OCc5ccccc5)cc4)CC[C@@H]32)N1 ; O=C1CN2c3c(cccc3[C@@H]3CN(CCCOc4ccc(Oc5ccccc5)cc4)CC[C@@H]32)N1] 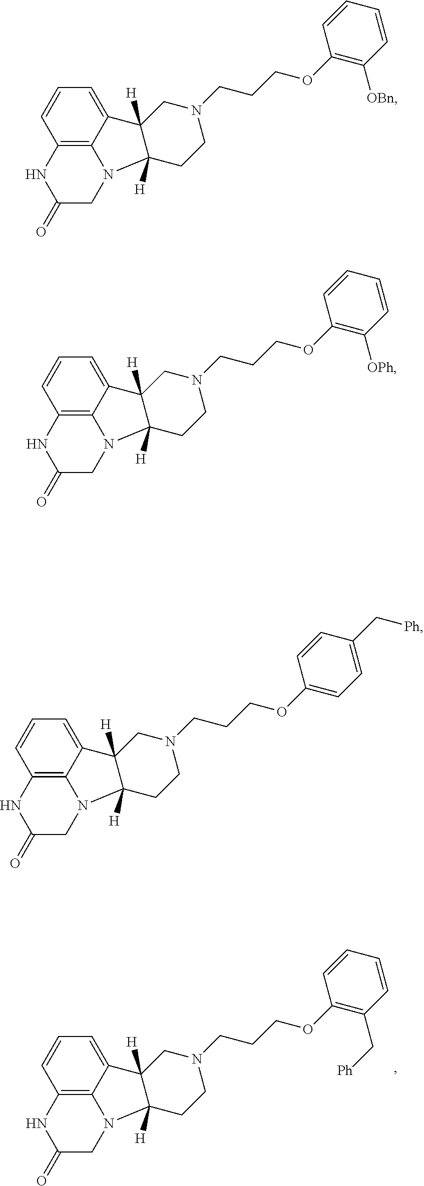 [SMILES: O=C1CN2c3c(cccc3[C@@H]3CN(CCCOc4ccccc4OCc4ccccc4)CC[C@@H]32)N1 ; O=C1CN2c3c(cccc3[C@@H]3CN(CCCOc4ccccc4Oc4ccccc4)CC[C@@H]32)N1 ; O=C1CN2c3c(cccc3[C@@H]3CN(CCCOc4ccc(Cc5ccccc5)cc4)CC[C@@H]32)N1 ; O=C1CN2c3c(cccc3[C@@H]3CN(CCCOc4ccccc4Cc4ccccc4)CC[C@@H]32)N1] 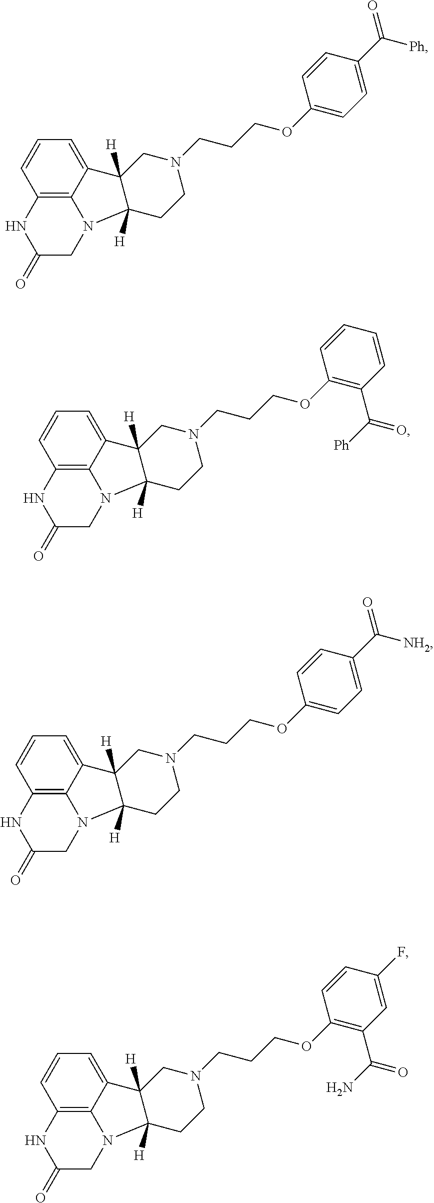 [SMILES: O=C1CN2c3c(cccc3[C@@H]3CN(CCCOc4ccc(C(=O)c5ccccc5)cc4)CC[C@@H]32)N1 ; O=C1CN2c3c(cccc3[C@@H]3CN(CCCOc4ccccc4C(=O)c4ccccc4)CC[C@@H]32)N1 ; NC(=O)c1ccc(OCCCN2CC[C@H]3[C@@H](C2)c2cccc4c2N3CC(=O)N4)cc1 ; NC(=O)c1cc(F)ccc1OCCCN1CC[C@H]2[C@@H](C1)c1cccc3c1N2CC(=O)N3] 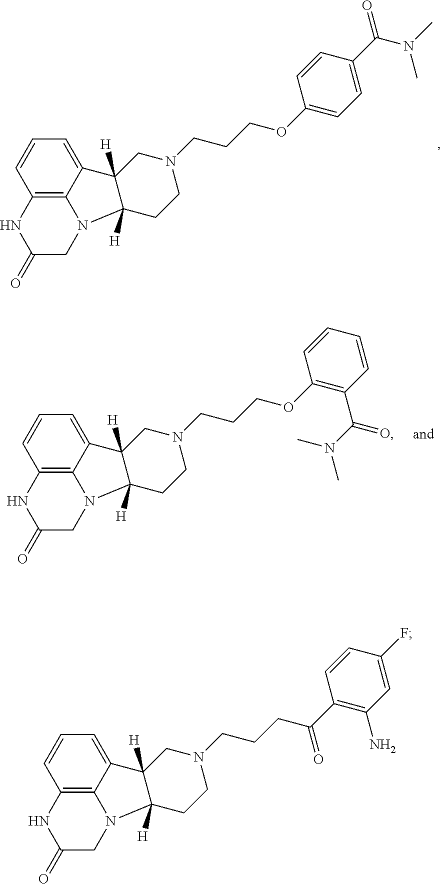 [SMILES: CN(C)C(=O)c1ccc(OCCCN2CC[C@H]3[C@@H](C2)c2cccc4c2N3CC(=O)N4)cc1 ; CN(C)C(=O)c1ccccc1OCCCN1CC[C@H]2[C@@H](C1)c1cccc3c1N2CC(=O)N3 ; Nc1cc(F)ccc1C(=O)CCCN1CC[C@H]2[C@@H](C1)c1cccc3c1N2CC(=O)N3]
each independently in free or pharmaceutically acceptable salt or form.

6. The compound according to claim 27, wherein the compound is a compound of a Formula II: 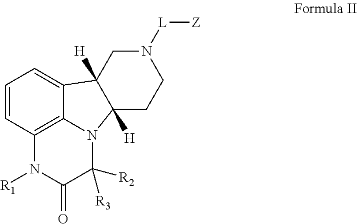
wherein:
R¹ is H, C₁₋₆alkyl, —C(O)—O—C(R^{a})(R^{b})(R^{c}), —C(O)—O—CH₂—O—C(R^{a})(R^{b})(R^{c}) or —C(R⁶)(R⁷)—O—C(O)—R⁸;
R² and R³ are independently selected from H, D, C₁₋₆alkyl (e.g., methyl), C₁₋₆alkoxy (e.g., methoxy), halo (e.g., F), cyano, or hydroxy;
or wherein R² and R³ and the carbon to which they are attached collectively form a group —CH₂CH₂—, or
wherein R² and R³ and the carbon to which they are attached are absent;
L is C₃ alkylene (e.g., propylene), C₃alkoxy (e.g., propoxy), C₃alkylsulfonyl (e.g., —CH₂CH₂CH₂S(O)₂—), or —C₂₋₄alkyl-C(O)— (e.g., 4-butanoyl); and
Z is selected from aryl (e.g., phenyl, naphthyl) and heteroaryl (e.g., bicyclic heteroaryl or monocyclic 5-membered heteroaryl), each of which is optionally substituted with one or more R⁴ moieties, optionally wherein Z is unsubstituted;
provided that L-Z is selected from:
—CH₂CH₂CH₂O-Ph;
—CH₂CH₂CH₂-(heteroaryl), wherein said heteroaryl is a bicyclic heteroaryl disubstituted with two R⁴ moieties;
—CH₂CH₂CH₂-(heteroaryl), wherein said heteroaryl is a quinazolinyl substituted with one, two or three R⁴ moieties;
—CH₂CH₂CH₂O-(aryl), wherein said aryl is phenyl or napthyl, and said aryl is monosubstituted with a hydroxy group;
—CH₂CH₂CH₂O-(aryl), wherein said aryl is phenyl or napthyl, and said aryl is substituted with two or more R⁴ moieties;
(CH₂)n-C(O)-(heteroaryl), wherein n is an integer selected from 2, 3 and 4, and wherein said heteroaryl is a monocyclic 5-membered heteroaryl substituted with one to three R⁴ moieties;
—CH₂CH₂CH₂S(O)₂-(aryl), and wherein said aryl is phenyl or napthyl, and said aryl is substituted by one or two R⁴ moieties; and
—CH₂CH₂CH₂O-(aryl), wherein said aryl is phenyl or napthyl, and said aryl is substituted with one or more R⁴ moieties, provided that R² and R³ are F;
and wherein
each R⁴ is independently selected from C₁₋₆alkyl (e.g., methyl), C₁₋₆alkoxy (e.g., methoxy), halo (e.g., F), cyano, hydroxy, amino (—NH₂), C₁₋₆alkylaryl (e.g., benzyl), C₁₋₆alkoxyaryl (e.g., benzyloxy), aryloxy (e.g., phenoxy), —C(O)-aryl, —C(O)—C₁₋₆alkyl, —C(O)OH, —C(O)NH₂, —C(O)NH(C₁₋₆alkyl), and C(O)N(C₁₋₆alkyl)(C₁₋₆alkyl);
R⁸ is —C(R^{a})(R^{b})(R^{c}), —O—C(R^{a})(R^{b})(R^{c}), or —N(R^{d})(R^{e});
R^{a}, R^{b} and R^{c} are each independently selected from H and C₁₋₂₄alkyl;
R^{d} and R^{e} are each independently selected from H and C₁₋₂₄alkyl;
R⁶ and R⁷ are each independently selected from H, C₁₋₆alkyl, carboxy and C₁₋₆alkoxycarbonyl;
in free or salt form (e.g., pharmaceutically acceptable salt form), for example in an isolated or purified free or salt form (e.g., pharmaceutically acceptable salt form)

7. The compound according to claim 6, wherein n is 3 and wherein R¹, R² and R³ are H.

8. The compound according to claim 6, wherein L is —(CH₂)n-C(O)-(heteroaryl), wherein n is an integer selected from 2, 3 and 4, and wherein said heteroaryl is a monocyclic 5-membered heteroaryl substituted with one to three R⁴ moieties.

9. The compound according to claim 6, wherein said heteroaryl is selected from thiophenyl, furanyl, pyrrolyl, imidazolyl, oxazolyl, isoxazolyl, thiazolyl, triazolyl, and tetrazolyl.

10. The compound according to claim 6, wherein said heteroaryl is thiophenyl (e.g., 5-substituted-2-thiophenyl or 4-substituted-3-thiophenyl).

11. The compound according to claim 6, wherein each R⁴ moiety is independently selected from C₁₋₆alkyl (e.g., methyl), C₁₋₆alkoxy (e.g., methoxy), halo (e.g., F), cyano, hydroxy, amino (—NH₂), C₁₋₆alkylaryl (e.g., benzyl), C₁₋₆alkoxyaryl (e.g., benzyloxy), aryloxy (e.g., phenoxy), —C(O)-aryl, —C(O)—C₁₋₆alkyl, —C(O)OH, —C(O)NH₂, —C(O)NH(C₁₋₆alkyl), and C(O)N(C₁₋₆alkyl)(C₁₋₆alkyl).

12. The compound according to claim 6, wherein each R⁴ moiety is independently selected from methyl, ethyl, propyl, isopropyl, n-butyl, sec-butyl, t-butyl, methoxy, ethoxy, hydroxy, halo (e.g., fluoro, chloro, bromo or iodo), and cyano.

13. The compound according to claim 6, wherein said thiophenyl is 5-substituted-2-thiophenyl or 4-substituted-3-thiophenyl).

14. The compound according to claim 6, wherein the compound is selected from the group consisting of: 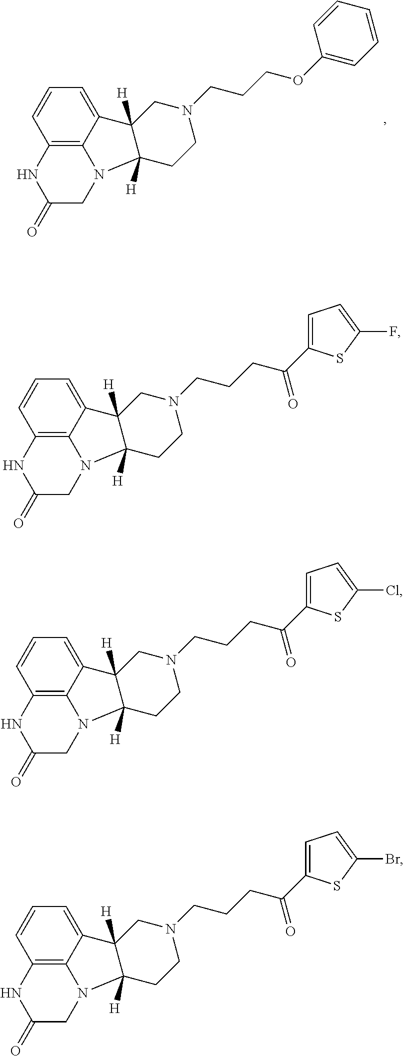 [SMILES: O=C1CN2c3c(cccc3[C@@H]3CN(CCCOc4ccccc4)CC[C@@H]32)N1 ; O=C1CN2c3c(cccc3[C@@H]3CN(CCCC(=O)c4ccc(F)s4)CC[C@@H]32)N1 ; O=C1CN2c3c(cccc3[C@@H]3CN(CCCC(=O)c4ccc(Cl)s4)CC[C@@H]32)N1 ; O=C1CN2c3c(cccc3[C@@H]3CN(CCCC(=O)c4ccc(Br)s4)CC[C@@H]32)N1] 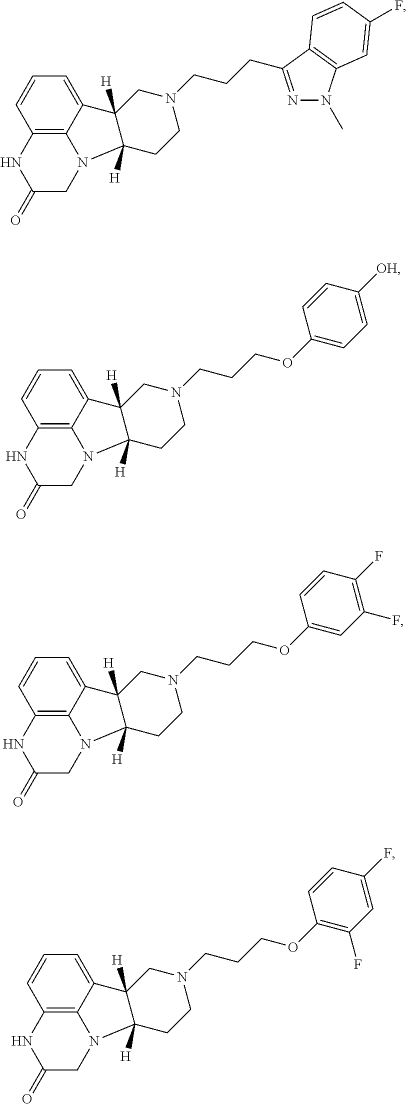 [SMILES: Cn1nc(CCCN2CC[C@H]3[C@@H](C2)c2cccc4c2N3CC(=O)N4)c2ccc(F)cc21 ; O=C1CN2c3c(cccc3[C@@H]3CN(CCCOc4ccc(O)cc4)CC[C@@H]32)N1 ; O=C1CN2c3c(cccc3[C@@H]3CN(CCCOc4ccc(F)c(F)c4)CC[C@@H]32)N1 ; O=C1CN2c3c(cccc3[C@@H]3CN(CCCOc4ccc(F)cc4F)CC[C@@H]32)N1] 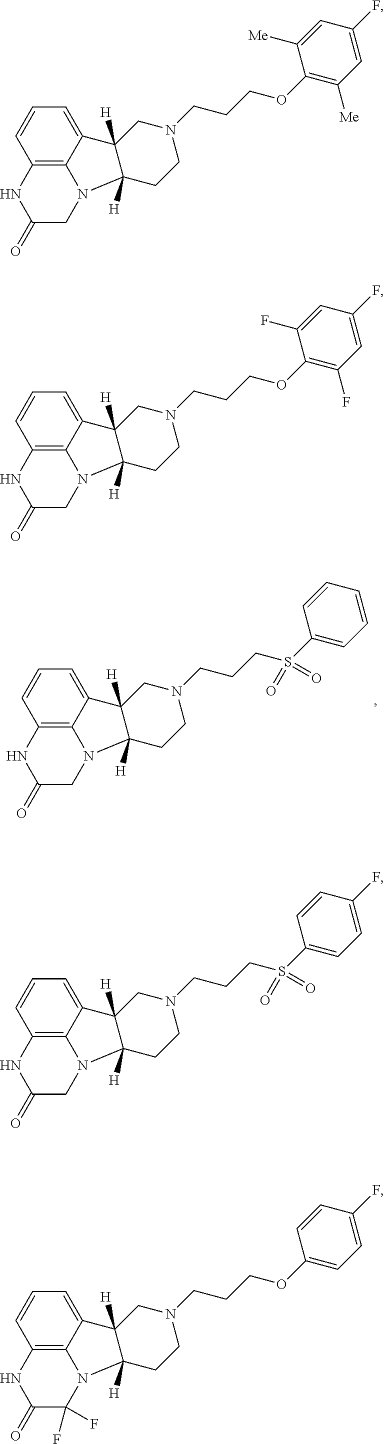 [SMILES: Cc1cc(F)cc(C)c1OCCCN1CC[C@H]2[C@@H](C1)c1cccc3c1N2CC(=O)N3 ; O=C1CN2c3c(cccc3[C@@H]3CN(CCCOc4c(F)cc(F)cc4F)CC[C@@H]32)N1 ; O=C1CN2c3c(cccc3[C@@H]3CN(CCCS(=O)(=O)c4ccccc4)CC[C@@H]32)N1 ; O=C1CN2c3c(cccc3[C@@H]3CN(CCCS(=O)(=O)c4ccc(F)cc4)CC[C@@H]32)N1 ; O=C1Nc2cccc3c2N([C@H]2CCN(CCCOc4ccc(F)cc4)C[C@@H]32)C1(F)F] 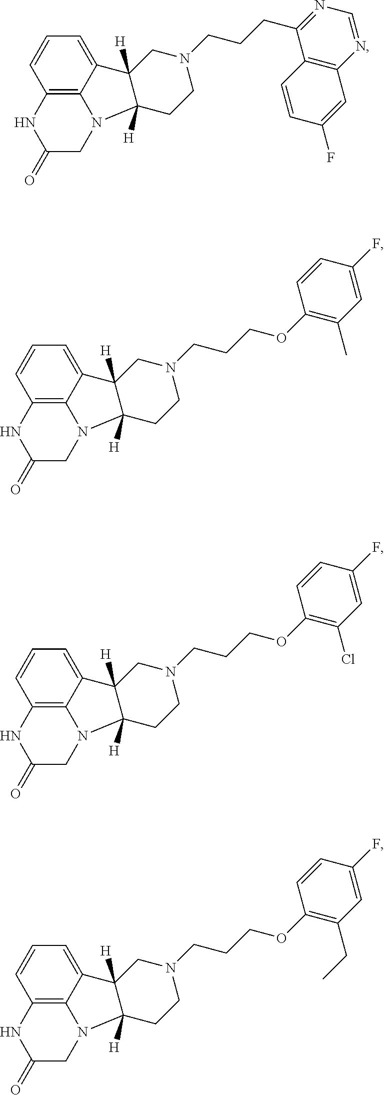 [SMILES: O=C1CN2c3c(cccc3[C@@H]3CN(CCCc4ncnc5cc(F)ccc45)CC[C@@H]32)N1 ; Cc1cc(F)ccc1OCCCN1CC[C@H]2[C@@H](C1)c1cccc3c1N2CC(=O)N3 ; O=C1CN2c3c(cccc3[C@@H]3CN(CCCOc4ccc(F)cc4Cl)CC[C@@H]32)N1 ; CCc1cc(F)ccc1OCCCN1CC[C@H]2[C@@H](C1)c1cccc3c1N2CC(=O)N3] 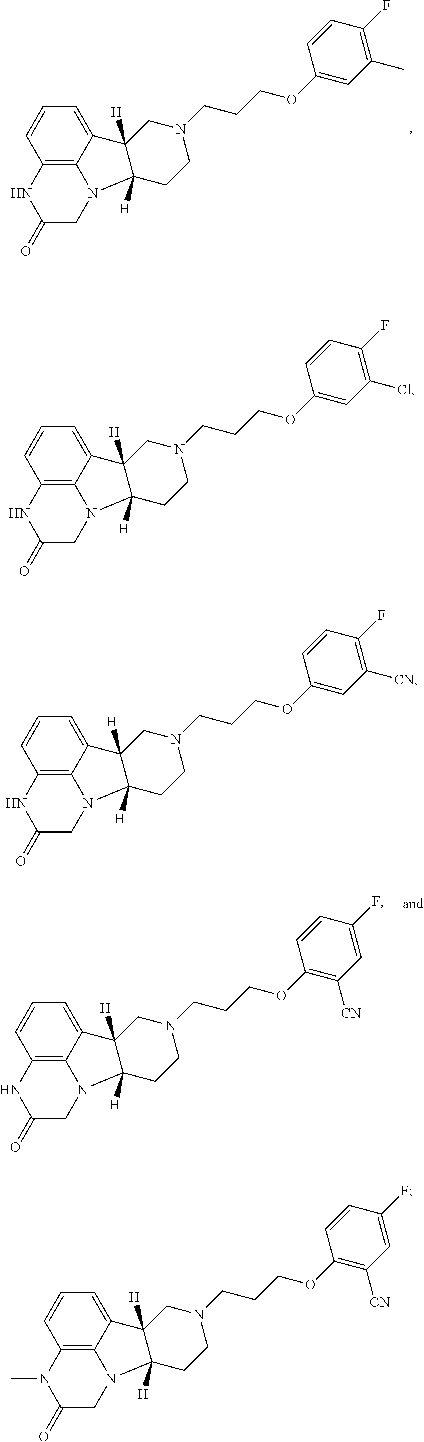
each independently in free or pharmaceutically acceptable salt or form.

15. The compound according to claim 27, wherein the compound is a compound of a Formula III: 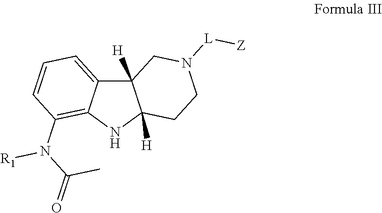
wherein:
R¹ is H, C₁₋₆alkyl, —C(O)—O—C(R^{a})(R^{b})(R^{c}), —C(O)—O—CH₂—O—C(R^{a})(R^{b})(R^{c}) or —C(R⁶)(R⁷)—O—C(O)—R⁸;
L is C₁₋₆alkylene (e.g., ethylene, propylene, or butylene), C₁₋₆alkoxy (e.g., propoxy or butoxy), C₂₋₃alkoxyC₁₋₃alkylene (e.g., CH₂CH₂OCH₂), C₁₋₆alkylamino or N—C₁₋₆alkyl C₁₋₆alkylamino (e.g., propylamino or N-methylpropylamino), C₁₋₆alkylthio (e.g., —CH₂CH₂CH₂S—), C₁₋₆alkylsulfonyl (e.g., —CH₂CH₂CH₂S(O)₂—), —C₁₋₆alkyl-C(O)— (e.g., 4-butanoyl), or —C₁₋₆alkyl-C(OH)— (e.g., 4-butanolyl), each of which is optionally substituted with one or more R⁴ moieties;
each R⁴ is independently selected from C₁₋₆alkyl (e.g., methyl), C₁₋₆alkoxy (e.g., methoxy), halo (e.g., F), cyano, hydroxy, amino (—NH₂), C₁₋₆alkylaryl (e.g., benzyl), C₁₋₆alkoxyaryl (e.g., benzyloxy), aryloxy (e.g., phenoxy), —C(O)-aryl, —C(O)—C₁₋₆alkyl, —C(O)OH, —C(O)NH₂, —C(O)NH(C₁₋₆alkyl), and C(O)N(C₁₋₆alkyl)(C₁₋₆alkyl);
Z is selected from aryl (e.g., phenyl) and heteroaryl (e.g., thiophenyl, furanyl, pyridyl, indazolyl, benzimidazolyl, benzisoxazolyl), methyl or OH, each optionally substituted with one or more R⁴ moieties, optionally wherein Z is unsubstituted;
R⁸ is —C(R^{a})(R^{b})(R^{c}), —O—C(R^{a})(R^{b})(R^{c}), or —N(R^{d})(R^{e});
R^{a}, R^{b} and R^{c} are each independently selected from H and C₁₋₂₄alkyl;
R^{d} and R^{e} are each independently selected from H and C₁₋₂₄alkyl;
R⁶ and R⁷ are each independently selected from H, C₁₋₆alkyl, carboxy and C₁₋₆alkoxycarbonyl;
in free or salt form (e.g., pharmaceutically acceptable salt form), for example in an isolated or purified free or salt form (e.g., pharmaceutically acceptable salt form).

16. (canceled)

17. (canceled)

18. (canceled)

19. (canceled)

20. (canceled)

21. The compound according to claim 15, wherein R¹ is H, L is —(CH₂)ₙ—X—, and wherein n is 3, and X is O, and Z is phenyl substituted with two R⁴ moieties each independently selected from methyl, halo (e.g., fluoro, chloro, bromo or iodo), and cyano.

22. The compound according to claim 15, wherein the compound is selected from the group consisting of: 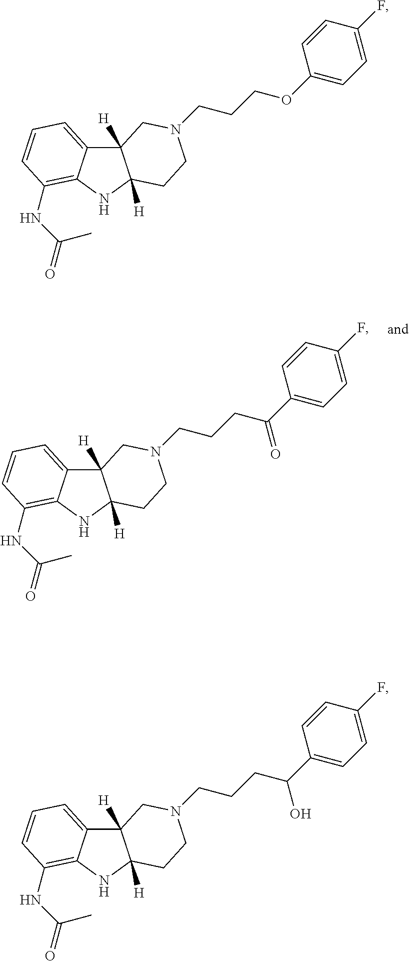 [SMILES: CC(=O)Nc1cccc2c1N[C@H]1CCN(CCCOc3ccc(F)cc3)C[C@@H]21 ; CC(=O)Nc1cccc2c1N[C@H]1CCN(CCCC(=O)c3ccc(F)cc3)C[C@@H]21 ; CC(=O)Nc1cccc2c1N[C@H]1CCN(CCCC(O)c3ccc(F)cc3)C[C@@H]21]
each independently in free or pharmaceutically acceptable salt or form.

23. A compound according to claim 27 in the form of a salt, e.g., in the form of a pharmaceutically acceptable salt.

24. A pharmaceutical composition comprising a compound according to claim 27, in free or pharmaceutically acceptable salt form (e.g., pharmaceutically acceptable salt form), in admixture with a pharmaceutically acceptable diluent or carrier.

25. A method for the treatment or prophylaxis of a central nervous system disorder, comprising administering to a patient in need thereof a compound according to claim 27.

26. (canceled)

27. A compound of Formula I, or a compound of Formula II, or a compound of Formula III, in free or salt form (e.g., pharmaceutically acceptable salt form).
